SEQUENCE LISTING (27 sequences)

SEQ ID NO: 1 - DNA, 48771 bp, Artificial Sequence
  BCAT1 cg21172322-cg23792314
cggggtaacctacgtgacgctcaccatgataccgtgcgctcctctccaggacccaggcaaacacaaaaaaggaggctcagacaaccaagcacccaggcccgaacctccaacaagcgtgtcttgggagcgctgccctgcacttcccaccctgccggggtcgcggcggtttttgtcctcctccgccggctcagggaagactggttaaattccaggtcagccctacagagccagggttcgccggcaaagaacaaaaaaacaattgtctcccttatatccgagcaaatagtctagactggggtgttaagccatttatagaaaaatctccagggcgcgctcagcctcgtggtcttgtcaatcacagacgcacaatagcaagcctgcaaagggaacggggacgggcgtgaaccatttcctccaccagcagggtcctccgatgccgcagcatccaccccacaccttaaacctcatggtattagtgggcaatttaaaagataaagacacagggaagcgggactaattgggaaaacctgcagacatttgttttaatgcgtaatctgctaaataactacgggggtgggggtggggaaggaagagatccaaggaggcagaaggctgcggtcaaaatattttggggtggcagagtcacgtaggatgtggctgtgggttctggcagcccagagattcagctcccgcctcctccctcagagcgagtccatagctaccctcacgtcccccgtggcggtcctcgccacgctccggagcgggttacccatgagggtgctagacctgggcagcgggaacctcgaagaggtggagattgcaggctgggactccagatttcgggcagggatgcggggaagggaagacgcctcgctggaggcggaatggagggcaaggcgaaggaggatggtgcaggaaacggcgacaaggcgcccggccaggcccgcgagctaccgagacccgggttccaatcctccccccttccgcaaacgcccgggttcgaggtacctggcgggcaagggccgcagcggagcgaagcgggctggccatggggaggctgcggggacgcggggctgcagagagcggcagtggcacggagcgcgcggctggaagcgaaagcaggcggtgtggccaagccccggcgcacggcccatagggcgctgggtaccacgacctggggccgcgcgccagggccaggcgcagggtacgacgcaacccctccagcatcccttggggaggagcctccaaccgtctcgtcccagtctgtctgcagtcgctaaaaccgaagcggttgtccctgtcaccggggtcgcttgcggaggcccgagaatgcgcgccacgaacgagcgcctttccaagcgcagatatttcgcgagcatccttgtttattaaacaacctctaggtgaatggccgggaagcgcccctcggtcaaggctaaggaaacctcggagaaactacattagggcagcttttccaccgactccaaatccaactgacaaaaagcagtttctgccctcgagagtttgcgggcggggattgacatttgtgcgtctgctcttgtctgccactgaccgctatgtgcaaactgaagggggagaacgtgaatccagcttttagatttccctgcgccacctacccaaaccgaatttgtaactcggggtgttatggggctaccaggctcgcattccctaagggccatttctgcccaaagatctcaatgcctttcatcgttttcaggcaaagcagaccatcaagagctccaatcatactgttttcatagttttccgatgtaggctcgtgatcgcaatatttagaaagaggactggaaaagtgatgttagaagtactattcggtttagaaagggaaaggaggattggaatagctattgtcttatatgcagtgttcgcctggggcaacgtcagcctaaattatgagccttcctggtttttaaattaataggaagtggtaactggggctgacttgatcttggaaagagggggagggcagtttattctgggtgaaagcggttaaatccggtttggttttttaaatggtttcatacaacgctactgataatatactgtagctctaatcttatcaactcagaaaacctacacttttcctctcctttatacaaggcacagaaaggcctcttacgctggggtggggtcccaagctccaaagaccacagagtccaggcaggtcacgtaccaccatagagcggcgagtgtccctggaagtccagggtcgcttataagataagttttgtccttgttgttttgagacggagtctcgctctgtcgcccaggctggagtgcagtggcgcgatctcgtctaattgcaacatccgcctccccggttcaagcaattctcccatctcagcctccccagtagccgggactacaggcctgcgccaccacgccgggctaatttttgtattttttgtagagaccgggttttgctatgttgcccaggctgctctcaaactcctggactcaagccacccacctatctcagcctcccaaagtgctaggattacaggcgtgagccacggcgcccggcctccatctgtattaactgcttctatttcctccccattaagggcttctgtccaattattccacctaaataaggtctctaatagccttcattttgttcctgccaatggttttgcttctcgtgcattttcatggctgcacctatgtgctgatgactcccaaatatattttttcagtccatctgtctcctgagcagtaggtacttgctactcaaaaatctgtctaaaataaaaacggtgtatctatccaccatgttcgaagcactgggctagttgctgggggagggttgagagcataccatcatcctgttatcatgcattgcccccagccagagagaaaagtgttaattgtgttagaaagtgttaaggaatcccacagacagatgtaaaattataacaagtgctgcctgtgctctaagagcctctaataatggattgtattgactcagaaaggcctggaaaggctttcagcaagtgatccttgagcagaaatctgaaagattaaagaaatttactaaaggaagaagggatgaagagcacctggtatgaaagtgagttccaggacaaaagaacttctctgaggagggctggaacaaagggccaaaagagagcctggggctgcggtagacagaagaggatggagcttgggctagtgagtagccaaagggccagccaagtagtgccttagggagagctaaagaataattttaaaaagcaaaaataaggtaaaagcctgactgtcatacaggtataaaataaactaaattatagagtttatttaaatcatttaatgaagttaccaggcagatataaaaactggttcaaagaacaggcatgtttataggtcagaaatattgaaatgaatatgcaaatgaaattggcttcttccacagtaggggagagaagttaattgaaccctgaccttcagattttacttgcatgactgcatgcaataattattttgcatttctatcagtcatctgtaaaataactcaaaaacaatgaaacaatgtaagacccaatgaaagggcccatggaatcagaatcagataaccttaaaggtttgctctaaataattcagttttccattgaagtacaaatttttccctacagtacggtaatataatttcttcattcaagaagtgctattagtcagcaacagctgaagtaaaccagacatagtagtcactgtacttactagttacactaagaagctgtagctttagcagttttcaatttagcttaacctaggggcaagagaaaccattgaaaggttaaggattaggtggagtatgcaggagcggtgacagaattgaaggtattggctgcagtttggagattggaagagggtctgggtggagaggaatggattttgaagaagctattacagaacccacatacgagatgatccttttaataagtcagatttggctggtggttggtaatagaagtggagagaaacagccagatttcagcaagaatggagataaggtggcagcctgggcaacatggcaaaaccctgtctctggtaaaaatacaaaaattagccgggctcggtggcatgtgcctgtagtcccagctactcacgggattaaggtgagaggattacctgaacttggtgaggttgaggctgcagtgggccaggatcacaccactgcacttgcctgggtgacacagtgagacaccatctcaaaaaaaaagaaaaagaaaaatagatgatataaggtgggacaaggatgagagaagtgccaaggaggcctccttggatctgacttgcttatctatttggtgatgttcactaagaaaaaaggaatgttgaaaaagaatgtgtttgaggggatgaggaggagtctggtttgggacctatgatttctgaagtgtctccaagactggctgtgagagatctgcctgtctgaagctcagagaagaggtctggattagaggtattattgaatcattggcatagaggtggtaactgaagctatgggtgcgaaagagaatctctatgtcagttttctccgctgtaaaatgagacaatagcaacacttcatgtttgtcaactgggatagatgagcattaatatatgaaacttatgtatattacttagaaccatgcctgatgtcagccatgcagaataggtgaaagaacgggcatggctctgtttccattaaactgtatttgaaaaacgggacagagcaaatgtggtctgtggactgtaagttgcttactcctgctctatgtagttgctctagtagaaacatagcagttgtccctcaaatgcatccaagtgatgatcaccaatgtctatccattcttgctccaaataccctgacagttgccatttctccaaccatgttatccgcatcctagtaccaaacagcttccaagcagctcctctgaccccagccttgcttctctttaagccattcttagcatgacaaccagtgaaaatgtctcaaacgcaaatctgaagacatctcctcttggtcaaaaaaccttcaaggcttcacatcacccatttgacaaaatgattggcaagctcccagcccatcccagattctgccttctgcctctgactctccagctcaccttttgctaggctctttctttctctctccccccacacatttaatttatttcagtcctttcggacatcaggccttctcacaggtgcccaaatacctgtaacattccctgcctttgccaggcactgtcatccttccttgttcagtccctgtgccatgtccttggcaggaacgtctctgaaccctgtctaggctacagtgggttcctctgctgtttatttacatacaatgcttgccatttcaaaataatggtaatattttattctaatcatttgattaatgctcttctaattttatgatgggaagttcatgtctgtcaggcctctgagcccaagctaatccatcatatcccctgtgacctgcacgtatacatccagatggcctgaagcaactgaagatcaacaaaagtgaaaatagcaggttcctgccttaactgatgacattccactgttgtgatttgttcctgccccaccctaaccgatcaattgactttgtgacaatacaccctccctgcccttgcgataatgtactttgtgatatttccccacccttgtgaatgcactttgtatgatacaccctccccacccttgagaaggtactttgtaatatccttccctgcccttaaggtactttgtaatattctcctcgcccttgagaatgtactttgtaagatccatcccctgcccacaaaaaattgttcctactccaccgcctatcccaaacctctaagaactaattataatcccaccaccctttgctgactctcttttcagactcggcccgcctgtacccaggtgattaaaaagctttattgctcacacaaagcctgtttggtggtctcttcacacgaacacacatgacaatgcctatattattcacctagccatcccacctcacccagtgccggacatgctgtggagagttaaaaatctgttgaactgcagctctctaatattttagaaagcatgtacttaatatactttggaattttaactgccaaagtttagaatataatagactaacatgttctcagggtccactccactcctagatatgagcttcttttcttcatagaactgctctccctcaccctaaatctttcttcttccttttttcctattttctgccttcctctactcgttgtcttcccttgtacagtcataataaggactttatatgcattgtgactgttcaaagagattggatcaatgctttccagtagaactttctgtgctgatggaaacgttctatatctgccaggtagccactaagccacatgtagctactgagcacttcaagtgtggttagtgtgattgagaaaccgaatttttaatcttgtttcattttaatttatatttaaatgtaaatagctgggccgggcatggtggctcacacctgtaatccccccactttaggagggtgaggcaagtgggcaggtcacttgagctcaggagttcaagaccagcctgggcaacatggtgaaactcacctctaccaagaacacaacaaattagttgggcgtggtgccatacacctgtggtcccagctacttgggaggctgagatgagaggatcacttgagcccaggaggtggaggttgcagtgagccaagatcacaccactccactccagcctaggtgacagagcaagaccctgtcttaaaaaaaataataaacattaaaaattttttcaaatgtaaatagctaactacatggggctacagtctgagcaccggtttgggcattgaagtcccttggttgagattcgttatcctggttctatcacttataagccctatgacttttggcaaattactaacctcttttgagtctcatatttaaactcagagtaagtaatgggcagtgacgacactcaacatgaagatatctataaaggacttagcagtacttagcacttagtaactgctcagtacatgttagctagtgttatataccatgttttaatttttttaagacccatttatttttcagttatatgtagcccttggattacggtatactgatgattaggatattatgtgagagaaaccttaaaaatatacatctagtgtttctaggtttttgtttgtttaataaagtttctttaaaaaaaataaggcccagtttggttcagaaaacaaactgcacagctattatttacatgcttcctggttggtattgggaagacaatgacagatgagtataattcctacctgccaaaccctccccttactcctgccatgtcacgtctctggaaaagtgagccataaaaaaatcttgacaaaagaattctcatttgggcttattcaatgccaagtcccattcaacgaagtaaaatgagcaattctatgtactgtgtatgtagttactgtatcatttgcctaggacggtagtaacaaagtgccacaaactgggtagcttaaacccacagaaagtgattgtctcatagttccagaggtgaggtgtggacaggcccagcttcctttggaacctgtaggggagaatcctacctttcttcttctagcttctggtaacccaggtgtttcttggcttgcagctgccaacacttcaatctctccttctgttgcattgtgttttcccttcctgtctgtctgcttcctgtaactgctttgattaatggactatggagaaatatggaagtgacactgttctaagactaagcatatacactaactggccaggcagtttctgctttcttcctcttggaaggatcttggaactaccccacctagaacccagtcaccccagctataaatagcccaaatcacatggaggagccacgtgcaggctcattagtcaacagtgcaacatccagcatcactgtcagccctgtgagtggctgtcttaaacatccagcccagtcaagccttcagatgatgccggcccctcatgactgattaaaaacacatgagagaccataaggctgactcagactgctcagccgggcccaggcaatctagagaccatgagagataaaaataaattattgttggccatgcacagtggctcacacctgtaatcccagcactttgggaggccaaagcaggcatatcacctgagatcaggagttcgagaccaacctggccaacatggtgaaaccccgtctctactaaaaataaaggaaaaattagctgggcttgatggcgggtgcctgtaatcccagctctacgggaggctgaggcaggaggatcacttgaacccaggaggtggaggttgcagtgagccgagatcatgccgctgcactccagcctgggtgacatagcgagactctgtctcaaaaataaataaataaataaataaataaataaataaataaataaataaattattgttttaaggccacaaagtttggggtggttggttatatagcaatgttaaccaaagcagcatgcaaacctaacatgcttatttcattatttatttaaaaacctaataataataaatcatgcccaaaaatagggataaaagggtgagataaacatgaaataagtaatcctttaaaatatctttctaactatggttattttattgtaccagccctagctaaaggcccagtatacacaagggccaagtaatcagtaatgacagtgatacaaatccttcttttaaagtcttcttgatgatcaaaattgtgtcacccactatttgtcagttccgatgaaatcattgttgtttttttaatctcatgatatggttaatggagaacttataataagaactggggccaggcacagtggcacacgcctgtaatcccagcactttgggaggccaaggcggccgatcacctgaggtcaggagtttgagaccagcctggccaacatggtaaaaccctgtctttactaaaaatacaaaaattagctgggcatggtggcgagtgcctgtaaacccagctactcaggaggctgagacatgagaattgtgtgaacctgggaggtggaggttgcagtgagccaagatcgcactactgactccagcctggtgacagagtgagactgtcttaaaaaaaaaaaaaaattgaaaaaatattagttatcatgaaaaacatgacacatttccttgttttacattactaatattttcattattattctggctttatccttttaaatttttatttatgtttgtttagagttttttttgcagacatctcttaaagccaatgtcccttttgcaaagattatcagttaacaccagatgacataatctgtaactctccttaaccaggctcacacagaccgccacaaatcacaaccctctggaagagaatgaatgcgcagggctacacaggcaaccccctgggagcaggagctatgccctcctctcccctcaggaccctgctcgaagtttgtgacagcgtggcagggtgacacacagaatgagcacgtgccaatctatgtgagttaaatacaaatacagtttaatctttcccacatgttagaggaaaaataaacataagttcttctgacattccagtggagtctgttgcatatctcaatatatacacaccacgatgaaactcactctgcgagcagacaaagtgcaaattcctgagcttagtctgtgggctcctgcacttgctgaatcacctgactccctcttccctccccacccctcgtcctcatttgcacttcccatcttcttctgcacagggaggaaaccctaagcgtggcaagcctctaggtcatctccaggtaccttaaaaaggagtgacagatggacagagagacaaacacatgaagattctggataaaaagattaggaatttcatttcctgtgtggaaaacaattaagcttataattttgcgttttacagaaacagaatcacttaacttctgaaaggagaaattaatcctaattaaatgaggctgcttttttaaaatccagatattatatactggattgctttggagaaaattttgttttataccagtacctaaatagcttttaagagttcaggttaacctatgctgaggaaattaatagcaaaaagaaaaggccacaatcaagacggaaaggatttaagttttattaatgattattaagtgcattatttatagtagaatccacaacatatgctcacgaaaataaaccagttctagtaaatacatgataaatataaaaaattagaagagggctgggcgcagtggctcacacctgtaatcccagcactttgggaggccgaggcgggcggatcacgaggtcaggagattgagaccatcctgtctaacacggtgaaaccccgtctctaccaaaaaagaaatacaaaaaaattagccgggcgtagtggtgggcacctgtagtcccagctactcgggaggctgaggcaggagaatggcgtgaaccagggaggcagagcttgcagtgagccaagattgcgccactgcactccagcctgggtgacagagtgagactccatctcagagaagagaaatggttgatgcctgacaatgagcaatatacatacacccaaaggagaaaatggggccgggcgtgatggcttatgtctgtaatcacagcactttgggaagctgaggtgagaggattgcttgagtccagaagcttgagactagcctggacaacacagtgagaccccatctctaaaaaaaaaaaaaaaaaatggagaacgtgggtatttgggaagacagtaatcttttggaaaaagtgtttttcctatgaatgtgatatatgttcaagaaaatagactgacttatctgatatataccttgtggcagcaaagggaatacttccccatcactttcttcagaaggttgctcaaaatcattgacaaggggcagattaataggagaaaagtcatacaaatttatttgatcataattttatgtgacacgagagcctacagaacgaagacccaaagatataagggaaactgtccatttttatggtgaaaccctgtctttacaaaaatacaaaaattttaatggacagtttttgttttgttttgtttgtttttttaagagcaaggagagtatgctttttaaatgccattggttcatgtgccacagaacctaaaacagcttcaaatggacaccaagtaaaaaataccagttttcagaagtctgtcattatgtatttacacaaattacataatcctgtatgtatttacaattacagattatcaagtagataacacaaagttgtagtgttaatgagacaaaatagaataaaaacaccacaaggaagccatttgcttatctaccaagcaggacctagcaaggccgatcctggcatgtgcttcaacattgcttggggacatttatgtgacagatgggaagatttgttcagtctgtgctaggtaagtctttgaccacagtgcaaacgttggcaccaagtagatggtagtttggctgtctgagtatctatttgtgaaactggaaccccccttccacgatggctcagggttgccatccaacaggctttgggccttttacaacaagaactggatgcaaacccatgagctggggatgactcactctattccccaacagacaatgcccaggccaaaaccctgagcccccttaggttaggttaaacaaagtatggagagccacgtagaaatatgattggacaaaaaaggtatggtctaatgctaatagactgagcagggaaatccagcacagcctgtctgtctggacccttcttgcctctctgagcatgcatttcttccttctgggtgtggtgctggaccctctctggaatgggggtcttataaccttcagtcaaacaaagtgagtcagataatttctttatggccttacacagacaggctgggggtggtggaagttagagtactatttttaggttttatggttggctttggggaaaaggggttttgtttctatggcccactttggggaagaggggttgtagtcttgatggctagcctcaagggagaatgaaaggtcagagacaggagagcaggagcccaacattccccagttgagaaattgggttggtagattatcttttttttttttttttgagatggggtctcactgtattgcccaggctagagtgcagtgtcataatctcagctcactgcaagctctgcctcccaggctcaggcaatcctcccacatccacctcccgaggagctgggaccacaggcacatgctactatgcccggctaattttttatatttttggtagagacagggtttcaccatgttgctctggctggtctcgaactcctgagctcaagcaatttgcccacctcagcctcccaaagtgctgagattacaggtgtgaactactgtgcccagcgggtggattattttataagccattgaactagtcttgcagtcgtgagaacaggccgctccaattaaacggttaacagttatatctcatttcaggcagtggtgttgcttccaccaaagtcaggcctctatgaatcaagcaatcagatgtttaataaaaggcatttccatgaaaacaaaagaaaaacaaagattaacatctggagttgtctataaactaattttcctagagtctctgaagtagcttcagattgcagtggcaatcttacagatatttctggattatagtttgaatcaggtgttcaagtaaaccttctgagtaatccatacatcagctgacatgaacactgctcatatattaagttgctgtcattatttcttccaaagtttatatcaagttgtctagctaaagcctgcagggcttggtgattccaagacagaaaaatggtagaaaaattaggaaacattagtttggagacttgtacccaggaaggaattcaggattcagcccaaattgtaggcaaataacaaaaactcaaaaaaacaattatcaagactagaatctaataacaagtatggtataattttcttctgaaatataattttctcttctacagtcatcccaacttttacccacaaagataataataataaaactaatttatttgcaaagtcagtttagtctctggcatgattatctgcttaaagtgcagtaagaatggtgatttatcatgaaggctcttttttttttgctttgatattttttatttattataaaaactgagttttcaacaaaggcagtttagcaggactatagttgtgaaaataaatctgaatgtagccattctttaaacttaaactaaaaatcattgtagaataaaatgtcaagcaagtgaaaacttttctgtgatatatacagaaatgatatagacataaatatcttcacataaacaaaagcaaagatcaagaaaaaaattaaatatcttggtgggcaagagaaatacacagattaaaaaggttatttttattttacttcaatgcttctattgagcacgcctgtcagagcaataggaattagataaatctttacatttcttcagggaattacatacaataaagacacctctctagaagaaaatatattagcatcattagactcctgaaagtcatgactttcaattaagttacactttttgctctaccgtgaagctacatgctttatcagaattttgccagttgaaagaaaataaagctaaccctggtaagatccagcacagacacaggtggcagcaaattaggcacaatgatgtctggattttcctctcaaagtggattacccatgcctaggagataacggcttgcaacgcaaaacaaacattgtggcataaaacagacgatatttaaatagatatattttctacagggatggctcttaagttggctttgttggaattttttcataaggaatctcagattaagacttttgaaacctctcaagggctgggcacagtggctcacgcctgtaatcccagcactttgggaggctgaggtgggtggatcacctgaggtcaggagttcaagaccaccctgggcaacatggtgaaacctcatctctaccaaaaatacaaaaaattagctgggagtagcaacacatgcctgtaatcccagctactcgggaggctgaagcaggagaatcacttgaatccaggaagcagaggtttcagtgagccaagatctcaccactgcactccagaatgggtgacagagtgagattccgtgtcaaaaaaacaaactcttgaagcaatgaagccaagccaaagattcgccatcagactgtgcctgtaaaacctgtatgaattgggtgaattgctctcttttcaacgtctccaaaatatcttgaggtccctgggcctgtcagaaagtgacattctttacttattgcaaagttagaaacgctataaaggaattgtgtgggcaaggtaccaggtgtgtctttttccaagtctactggctgtataaagtcaacctcaatccctcgaagcagtccggttgcactcaaaaaaaagacattccagtcaaagccttggtaaaataaccactgtttccatgtgtccagttacaaaagaaaataagctctttttttgtttgtttgctttttgtttttttgtttgtttgttttgagatagagtctcgctcttgtcccctaggctggagcgcagtggtgcgatctcggctcactgcaacctctgccacccaaggttcaagctattctcctgcctcagcctcccgagtagctgggattacaggcgcctgccaccgcgcctggttaatctttgtagttttagtagagacggggtttcaccatcttggccaggctgttctcaaactcctgaccttgtgatccaccggcctcgccctcccaaggtgctgggattacaggcatgagccaccatgcacagccgaaaataggttcttattgaacttatgcaaataactatattgccagaaaataagaacactaatgaatagtttccaaattctgaagaattcaggtagaaagaaaggtaaatgtttccattttgctcacagaagtatactttaccccaatgctgtaagctataaatagctcaaaagaaaaaaaatattttcttgagtctggaaaacaaaacataaaaaaaatcagtaatgtttcaaacaaaaaccctttaaaatagtaatttcaatccttcattcactcagtcccatgtaattctcgttctccttgatgttgggttagcaatctgcatgaatgcatcagtttttcattagagctttggactttttttttttttttttttttttttgaggctggagtgcagtggcgcgatctcggctcactgcaagctccgtcttgccgcttcgcgccattcttccgcctcagcctgccgggtagctggaactacaggcgcccgccaccacgcccggctaattttgtttttgtgtttttagtagagatggggtttcaccgtgttagccaggatggtctcgatctcctgacctcgtgatccgcctgccttggcctcccaaagtgctgggattacaggcgtgagccactgcgcccagacgagttttggatgtttttacctagtccaaaggtgtgatctccaaagttatcagaaacctgtatttaagactacttgtcaaggtccctttcatgaatttccttgaatacacagcacttcaggatttgcaaaaggcttttagaaaaaaaaatcagaataaagcaatttactgcatataccatgacatatcagccttttatttttattttcactttttatttttggagtaatgtggaactttttaatttggaaggcaaaaggttacagttaattgaaggcagaagtcaggttaataaatgttacaaagttgttctgacagagagagggaacttctctgggctctcctccacaccaaatcagttggtagtaagcacaaatttgaagaaaattcatgtgtcaaacaagctgccatctcaagaactcttaactcttatccaggaaaatcaaagtgagcattgttccagtctcttactcttcaattaagtaaatgggaatgattcagccaacaaagttcatgacgataaggtacaagatggtgctagcaaagagaaagaagcaaagtctcactccaaggagatgctttcgaagtccactttgttctgtgggttcacctgtattctcaggcaaactactaggatgaaactccccacccaagatgtgaaacccaagaggaaagagttgaaggggaaggtccccatgagaagacagtaagtaaactgcagcgccctagtcagcagtttatacagcaggtatatatccagcaacttcagacactgcagagtagagctaagtactcttctaagaaccatcgggtgcctgacactaccaacgctgacatgaatggatgcaaggtaaccggccagtgctccctaggctcatagaggacccaacaaccacactggatgtccatatcagatttttaggaatctcatacaatgttggaacacatattaacaacatatccatataaatataactcaaagaaagtgtaacaccatttcttatttaacaacacttcctgtatgattttaacataccaaataagcctcatgtctctcttggacttccaggggtcctatttattattaataatatatgttaatgtattatagtattatgttcaagttagttaagggcaaaaatacttaattttagaatatgaaatttgattttcagaagtatatcatatatcaaaagtttaaaacccttgctatcaaaataaagatttaagacctatgttcaaaatagaatcgcaagtcactgtgacctgatcgaggacctgggaactgactggtttcctaacacattcctgtcctgtgtacttctctggaaagtcttcctgtatccccaagagcacaagttttgcatggtagagatgctagtttaacattgcatgtaatagactagttatgacacttgattattaccctgcttgtttatgtgtcagcctccaactgtctaggcagtgacttcattctctttgggtttccagtgcttagtacacagtcagataaatgtgtgagcctcgataaatatctactcaataaattgattgaatatattaatctaccatgtttttgcttaaattaaaaattgatcaatgggctgatatattttataacacatgcatacacacataagtagagacagaaagagagacagagcagaaaatagataccagcataaagtcaattgtgttaggcacaaaagaaccaaattagattttaaatatggctatttattcaggctacttactgaactgcctttatctgtctatgagatactttgccttttctcctaacttcctcgcatttctggatagacagtgaaatgtccgcaggacttttcccaaatcttattaagccatgagttctgagctcattagaagattttgttttaagctgtagctccattgtccagctgggtgtaagttctagctttattgtcagatagacaattatctagtatctagtagataaagcttgggtaaaccacctaatgtctctaaaccccactttaatgtccctgaacctcggcctcctcccctgtaggagtcaaaaggaaatgcaaactactgaacagaatatgtcatggttatttgttctaacataaggttatcaagcttataggtagaccctataatataccagtaaatcacatagactctggagtgacattggcagatttaattccagcaataccacttactagctttgcaaggtttgtctaattacttactgatatgggttagctatgtctccacacaaatctcatcttgaattgtagctccacgtgttgtgggagggacccaatggaagataactgaatcatgggggcggtttcccccatatagtttctgtggtagtgaataattgtctcatgttacctgatggttttataaggggaaacccctttcgcttggttctcattttctgtcttgccaccaccaggtaagaagtgccttttgccttctgccatgattgtgaggcctcccagccatgtggaaatgtgagtccattaaacctctttttctgtataaattaccctgtcttgggtatgtctttattagcagcataaaaatggactaatacacttaccctctctgtgcttccatttctctatctgtaaaataatattaataatagagttttatgagaactaaatgagataaatcatgtaaagcctatttcagcatccaggaagatctcaatctgtgtcagttataattgttaagttgaagattcatattgagtctttagaccttgtgaggtaatatggcatgtttggaagctgcgttatttataattttagagagaagaatgaatccacaggaactccacctctctgcaaaccgcactctgtggcatggcttaattagaggcctgctgcagggctgagaagtaatgcttgaccttagatgctccataaatcctcttggcaaactgaccactccagatttttatttttattttcattttttatttttggggtagcatattttatttttggagtgtttctaccactatactaagaggggctgtttcccgaaaaactctcaaaccatttttcctctgctctcacacaaaaacaatcaacacggaagacttctggaccccaaaatatatagggatttctccccagcaggaagcaagcaatcagttctgcagtggacacaagctaggtgtcctccaattcagctccaacactgtctacccagagatagcatcagatccacaagttgagggctcagtcccacaagagcaccccgtccttccaaccagtcataagttcaggtctctggaactgctgaccaacaggtttcaagttggggttgccatgaccccctcttccggtttgattaatttgctagcgtggttcacagaactgaggaagacacttacatctaccagtttatcactcaggatattttttaaaatacaaataaaagccaatgaagagatacaaggtccaagtctggaagggttctgagcacaggagcttttgtcctcgtggagttgggatgtgccaccctcccagcatgtgaatgaatttttgcttaccttcccgtgaccctccacgtgttcagcacaggaggctcacgggaaggtaaaaaacacttcttagaagctccccagtccattccctttggattttcatggaggcttcattacataggcatgactgattaaaccactggccattggtgatcaacttgaccagccccactctgaagctatcagtcaacactaatatacaaaaagacagcactttggagattccaaggatttttagtacttgtatgccaggaaacaggaacaaagaccacatatatgtatttcacaatatcacagggacactggctttttttaggcctttcctcagatcgctagagcagcatacaggaaaaccagttttccactagggttccttagatgttcagacttggtgtgatcctccagtgagttaaaacatacctcctccaaaagtctgattcatttatctctctacttagaaaaaactgctatggaaactctttttcaccaatgatgatgacatacatttttcccagtaatgtaagcccaatatatgccagtctcaatttacaagacagatacatcataggatctttgatacacatacctaggaagatctttgaattacttaataaaacattcaggaatattccctccaccccagagctgacaaggaacaacagttatagaaaaatagttgtagaggcagtgaatatttaagatgggttggtaccttttgttttaatagcttccattagtcacaacttgataaactgtaacattcctttctaaatctgcttcctgctccttcctcacctctgtcattttctgcagcagaatatgtagtacacattggctctataaattgccaggtgcacatcaaaggaaagcatgaaaaaaatagtaatgaaaaagtaaacctcccaaaggacttcgtcgttagataagaaaactagcacacatgtgcacacatacacacatacacacacacatacacactttaatgaaagaactaggggtcctgataagctggcacataaacctgttgttcccatcaaatgaataatcaaagatttgagagttaatttgagtgactgtgtcagtattattccaacaaagacatgagcacacacgcaaacacaagattgttccttttttttgagacagactctcattacattgtccaggctggagtccagtggctattgatagacgtgatcatagcgtactacagccttgaactcctaggctcaagcgatccacctgcctcagtcccccaagtagctgggactacaggcatgtgccaccttgcccagctacaagatggtttttacctgaagagttaatatgatccgtaataataaccacccactgtatgactcatcctacatccggcccctagtttcacacatatttcaccatatttttcatacttaaaacaaccctataaggtagatatcatcattattatagcaacccattttacatatgagaaaattggaagcagtttggtgattctctaagttgattcacaggactcaatactttcgactataatttattacaggataaaaattatggccaggcacagtggcccaggcctataattccagcactttgggaggccgaggcaggcggatcacgaggtcaggagatcaagaccatcctggccaacatggtgaaaccccgtctctactaaaaatacaaaaattagccggtgtggcagcgcgcgcctgtagtcccagctactcgggaggctgaggcaggagaattgcttgaatccaggaggcagaggctgcagtgagccgacatggggccactgaactccagtctgggcaacagagtaagactctgtctctcaaaaaaaaaaaaaaaaatttacaaggcaaaaatcagcaaaaggaaaaggcacatgggacaaaggaaatcagacatgtgcttcccagagtcctctcccagtggagtcacacaggatgcacttaactcctccagcaatgagttgtgataatatgtgtaaaatattacttactagggaatctcattagacactcagtgcccaaggtttttatgtggggctgtcatgtaaacacccctacctagcacatcctaaaattccagactcccagaaggaagcagatgttcagcagaaacttcattgtttgtacaaatattttaggcatcgtgagccattcttttcagggaatggtgagaaccctccagaaattcaagttcccagacaccagacaagggccaactttgcaagcctttctaagactagcagcctcaggcctgctttaattcttttctacacaggagtagactgagaagccttggtcaactgtgcagaagcagaggacggggctgtcactgagcaagggacatgaagcattcaagtgtgtgtatatatatattttagacagggtctctctctgtcacccaggctcccaggctggagtgcagtggcacaatctcagatcactgcaacctctgcctcccaggattctcctgcctcaacctcctgagtagctgggactacaggcgtgcaccaccatgcctggctaattttttgtttgttttttgtagagttgaggttttgccatgttgcccagcctggtctcaaactcctgagcccaagcgatcctcccacctcggcttcccatagtgctggaattacaggcataagccaccatgcccggccttcaagtatatttcttatgctcaaatcagagagaaccaaactgagtatcaagaagattgattgccttgaccaaagtaacacacctgtctgcagagggagagctggtactagaacccaggtcttcagaccacacagggcaattaacaaatatcagagtgaaaagcagaatctgctcagtccatccccttctctgccattagtgaagatactgatcaattaatttaatttttattcatacactataaggatttattaaaaatcaaacaaaacaaaacaatacaagttgatttttcacaggtggctttttaatctgcatgatggcataattacagcactagacattttcaaccgattatgtcgataatatacattctcgtttcctccctttttttcccctcagctttctgacccatcctcccttttctagagtgagaagaccataaattaccaatgatttattgaaaaatactgcctttgagtaattcttagtaacttgtaagaaaataacagtaacaaattataatgagaggagggagagaaagagaaccccttctttacacaagaatgctactcaatgccaagaatgatataattagatcagttaatgctaaagtcatcagaagaaaggttgtcagagaatgggatatttacacagtctcaaagtcttgtgccacaaattacatattaatttaaaaggagaaaatgtccctttacaagagtgagaaatggtggcgaccaccttcaccagctaatcaaacttaacatgactaatgcaggaaccaatgcatatcatgtgtctttgatgcagggcaattaggagtacacaacctcacttcagtgttattcttgccaagaattagagcctggctctaatcataaagataaaattagagaaattcaggatgtgaaatagccaatgagcctgaattattcaaaacaatgtcattaaaaaaaatagcagcaaaaacacacaaatattgatccgtattaaaagaaacataactataaagagtaatatgtaaacaaaacaaacacattttgaggtcagctagggaaaattatatatgaaccagatatgacatgatctctctgaattaatactaatcttcttaggtgtgctatgtaggagaatgtcccatatgaagtatttagataggaaatatcacaaccataatttattttcatatgattcagtacttgggagaaagagaaagcatatagaaaggttaaccattgggtgaatccgttgataaatgacggattcaaaaacagttgttttttgaaacagtagttctttcatctctacagagtgatggttaatttcatgtgtctgcttggctaagctatagtgcccaattgcttagacaacacaagttcaaatgttgctgtgaaagtattttttagatgtgattaccatttaaatcagcagactttgagtaaagcagattgccctcaatgtgggagggcttcatccaattagttgaaggccttaacagcaaaaaagacggaggtttcctgaagaaggaatttggcctcaggactgcaattgcaatagatactgtacctgacctgccctgcaaatttcagactcacgactgcaacatcaactcttaccagaatttccagctgaccaacttgccctatggatttcagacttgccagctcccacaatcacatgaaccaattccttaaaataaatctctctctctcgatctcttgatagatagatagaaagattatagatatagatatagataaagatataaatatagatatagatatgccctattggttctgtttccctggagaaccctgcctaacacaataggtttggaaattgctaaataaaaaattgcagctccccctccccctctccctctccccacggtctccctctccctctctttccacggtctccctctgatgccaagccgaagctggactgtactgctgccatctctgctcactgcaacctccctgcctgattgtcctgcctcagcctgccgagtgcctgtgattgcaggcgcgcgccaccatgcctgactggttttcgtatttttttggtggagacggggtttcgctgtgttggccgggctggtctccagctcctaaccgggagtgatctgccagcctcggcctcccgaggtgccgggattgcagacggagtctcgttcactcagtgctcaatgttgcccaagctggagtgtagtggcgtgatctcggctcgctacaacctccacctcccagccgcctgccttggcctcccaaagtgccgagattgcagcctctgcccggccgccaccccatctgggaagtgaggagcttctctgcctggctgcccatcgtctgggatgtgaggagcccctctgcctggctgcccagtctgggaagtgaggagcgcctcttcccggctgccatcccgtctaggaagtgaggagcgtctctgcccggccgcccatcgtctgagatgtggggagcgcctctgccctgctgccccgtctgggaggtgaggagcgcctctgcccggccgccccgtctgagaagtgaggagcccctccacccggcagccgccccatctgagaagtaaagagcccctccgcccagcagccaccccgtctgggaagttgggggcagcccccgcctggccagccgccccgtccgggagggaggttgggggtgcctctgccaggccaccccttctgggaagtgaggagcccctctgcccggccgccaccccgtctgggaggtgtacccaacagctcattgagaacgggccatgatgacaatggcggttttgtggaatagaaaagggggaaatgtggggaaaagatagagaaatcagattgttgctgtgtctgtgtagaaagaagtagacataggagactccattttgttctgtactaagaaaaattcttctgccttgggatgctgttgatctatgaccttacccccaacccggtgctctctgaaacatgtgctgtgtccactcagggttaaatggattaagggcggtgcaagatgtgctttgttaaacagatgcttgaaggcagcatgctctttaagagtcatcaccactccctaatctcaagtacccagggacacaaacactgcggaaggccgcagggtcctctgcctaggaaaaccagagacctttgttcactcgtttatctgctgaccttccctccactattgtcctatgaccctgccaaatccccctctgcgagaaacacccaagaatgatcaattaaaaaaaaaaaaactgcagacaaatttttaaaatcctcacttgtatgttacatgtgggacagaataattttaaaaaacacatataatctccaaaccccagttcaactataagttataatcttttctccctgattaagatttgtatttgaaacaatagtcatttgctttgagttaaatttcatctcttggatgataatgaaatgagtaaaaatcaagttataagatgtctgtaatcccggctatttgggaaggtgaggtgggagaatcacttgaacccaggagatggaggcagcaatgagacgagattgtgccactgtactccagcctgggtgacaaagtgagactctgtctcgaaaaaaaaagaaaagaaaagaaaagaaaaaaatcaagttataagagagaaacagaagaatgcaaggtgatctacatagacaagctacatagcaatactggaaaaacctagaatgttatttttccttaaaaatctaccagtgactccaattaaacatggtagactgaacacatgaatctttctccacatctttttagatattcacttaaacaaataccattgtcttttaaagtttaaaccataagggaaagagaatgggaaacatgataatgagtgacattaacacattttggaaggtagaaagctgacagtagagaaggaactaactctgtagattagaaagaactgaaactgtattgcacaaagaaggagatactgatgagaagccacctttccctcatctcagaaagtctcagaatagagaatccaggtgtttccaaaggtgaggctgaaaacagggacttttgatgcaaatctatataaccattcattgaatgaccttgacttgggagagtacaaccagaaatagttttatttttctctcattactgcccttcacacccactttcaaaaatctaccatccctatctggagaagatcacactgatcctattgagtttccagtcacttttaagtattaggaaaatcttgctgcaagtacttaaaagaccggtatgatctgtaataaaactttgacctcacattgcattcaatgctttactgtctgagcagccaccaccaccagcccccagcaagctgaggttgttaacatctgtaaaagaaaacaaaattcttaaaactctctaaaattatttattatgccaagggagaagttaagtactggaaactgagtcacatagcatgtttgcaattctgcttcttaggttatagattcactctcttctcatcgttctagttctgtaaatgactaggagagaccagagaccagaccttcccccttcactgatctttgttatggattaatggattaactgcctcctttattgtcctgtacctaactcacaccagttggcacaaaagaccccatgggatgagcacagtagcccatgcctgtaatccaggcactttgggaggctgaggcaggagaattgtttgagtccaggagtttgagaccagcctgggcaaaagggggatctggtctctataagaaataaaaaataaattagccgggtgtggtggcacatgactgtggtcccagctagtcgggagactgaggtggtaggatcacttgagcccacaaaatcgaagctgcagcgagctgtgattgtgccatgccagcctgcaatacagagcgagacagtctcaaaaaaaaaaaaaaaaaaccaagaaaaacgaaaatacaaacccatgactgttacaccttcagtgtgaaatgtttgttgttgttgttgttgttgttgttgttgttgttgttgttgttgtttgagatggagtttcattcttgttgcccaggctggagtgcaatggtgcaatctcagctcaccacaacctctgcctcccaggttcaagcaattctcctgcctcagcttcccaagtagctgggattacaggcacgtgcaccatgcccggctaattttttttattttattttattttattgtattgtatttttagtagagaaggggtttccccatattggccaggctggcctcgaactcctgacctcagatgatctgcccgcctcggcctcccaaagtgctgagattacaggcatgaaccaccgctcccagcccgtgaaatgttaaatatgcctttctcaaatgaaagtgaccaccttgactaatcagatcattgtaactaggcattaagccttacacagaaagatgttgaaattctgttaagcttccctaaactctctctctataaacaatcccaaacttctaaagttcagaacgctgacttccattctttgggatccgtgtttccctagcactgtccttaaactttgcacttgaataaagtatctttaaactagattctgacccttttgattattttaggttggcacatccaacagaaatggggttcctgttttttcagcattggagctcaggacgcaccatcccaaaatatgaccagaatatgccacatcaggatatgcttatttgatgtatttccagttagttattctgagacactgtagacacaggagtagctctgaaaaactgtctttttgtaaacaaaatttatatctataaagaaaaaaactttgataagaatctcatcaaccagggaagattttatcaccagggagaagattggacaggtagaccttgtcacaggctattacctattcttctgatgggggactctgagacaacttttatcacctgagagactttttatctgcataacaagacaacctatgcttgctatacacttcctctcctcaccctcttataacctgtcaccacctcccctcaataagccccaagccctgattccttttctgtagctcaggaatactatataaacttcaatcatctggcctttctttgagtctcatattttatgggactcccatgtgtacgcacaaaataaatttatgtcttttctcctatttatctttctactgtcagtttacttcataaactcagttatgaaaagggtaaagaaagttttctctctcccacatcactttcctctccattctccagctgctgtttccaggacccacggggttaagtgtggaccatgaattagaggaaatggttaaaaatctcatttgtctgtgaccattataatctggcactgggggccctctcctatggcaaatgcccaaattctggctttctattgcatcatggtcacttacggattcttcacaacctcccctcccccttgtcctcacctcctgtggagaccactgccttgcacagccccactgcaatgatgcacattctgtctcacctctaaggattctccttcaaccccagctctggcattattccctacacagcctctgacccctggggtctccttgccccagcaagtagccttcttagacagtgtccttttgaggtgtttctatgacacctttcctcagagttcacatagttcatgacgtctttgctctactaaactctggaactgcaaactgcacccactgaagacccctctcttgactctaaccacaggcagcaccagccagccttgactgtcccagggcagagctgacactgctatgttccgtacaccagtggcacaagtcaagtgttcgcaagctacagatcctctaggcaaagcatttctcacttaatttttagtgaatgccctgttgggggaatgggagaatcacttttttacctcttccagggtaaagaagtttccagaaatgccaagctctccttgcttggctgacaattttttatttccaaataatcttatttcctttctgttcaatttccagccttctattaaatataaattggagataagttagaggctaaagcttgcagaattagtttagtcatcctttagaaggttctttagatgtgtcaaacgtgtttggaatttggaggtagtatccctccatgctccgcactgaagcctcagtggaacagggcccttttttttttttttttttttttgagatagagtcttgctctgtcacccaggttggagtacaatggcacacaatctcagctcactacaacctccacctcccaggttctggtgattctcctgtctcagcctcccaagtagctgggactacaggagcccaccaccacgcctggctaagtttttgtatttttagtagagacagggtttcatcatgttggccagactggtcttgagctcctgacctcaggtgatccactccccgctcagccttccaaagtgctgggattacaggtgtgagccactgcacctggcccaggatgctatttttttaaaggattaatcagaagaaatatgaagagtaaaattttaaaattcaggggaaaggctgaaagataacattgatgagatctttctcccacctcaaaaattggacactaggagagaaaaattaaattagaagatcaaacaaggaggtctaatgtcaaacaaatagaagtttaagaatgagaaaagagggaaagcagaggagacgaaatcatcaaagatataggaaaggaaaattttctaggatgaatgactaaggacccatgttatgataaagctccccaaattagcaataaaaagagaccctgaatacctcaaggaacaaaaaacaggttacacataaaaaggacctgaaatacgagcccctttggacttcctatcagtaacacaggaaactagacaacagaaaagcaattcccttaaaattctgacgaaaaatcatttctaacctaggatattattcaaaatcaagtattaacctagagcaaaagtagaacaaaaagtcaggcatgtaaggaatcaaaacatctacctctgatgcactctttatctaaaaacaactagagcatacgtgcaacacaaatgaggaagtaaagtaaaaaggagaacgccacacgattcagaaaaccggggatgtaacacagagagagggtaaaagatgccaagaggaagtcttaggcaggcagctgtgcagctggctcagagagaaaccagcacaaattagtgcaagaatgagaatggtagcagaaagagtacagagacaaaaaagaaaatgaactgtcagatacatttaacctaagtaaaattacattgagaggctgtcaggaagttgggaattggaaagaattcataataaatatatagtaaggtaagtaaataaaaggaaataatgcaagaattaactgcaggaaaaacaaaatgaggtataaggggaaaaaaatgtaaccatagtacactacttgactcaccagtgacttttaaaaatggaattccatctactactcaaaacctctcaggggcatttcaccacacttagaataaaatccacacttaccaggactaaggaacttcaatgatgtggctccactcctgaaatctcccctctcagattatgctctgggaagtttcatttgctgttccatatgcatgggacactcttacctccagaccatggtactcaggtgtcagctgaaagttcaccttttctgtgaagctttccctgaccagtcagtataaatttgtaccagctttctctcagttctgtcactactgcatcatttcattttattcatattgcatcactgactgaaaatatttgatatgttactatatgaggataaattgtaataatattagttaatacttacagtacttacatagtacttatttcagaccagaccctgttataagtgctttatatttgtttttggatctttgttgcagactgtctccttctatcgcacaggctggagtgcagtggcacaatcttaactcactgcaacctccacctcctgggttcaagtgattctcctgcctcagcctcctgagtagctgggattacaggtgtgtgccaccatgcctggttaatttttttggtttttttgttttgttttgagatggagtctcgctccgtcgcccaggctggagtgcagtggcgcaacctcggctcactgcaacttctgcctcccaggttcaagtgattctcctgcctcagcctcccgagtagctgggactacaggtgcccaccaccacacttggctaatttttgtatttttagtagagacggggattcaccatattggccaggctggtctcaaactcctgaccttgtgatccacccgcctcggcctcccaaagtgctgggattacaagcatgagccaccacgcctggcctttttttttttttttttttttttttttttttgtattttaagcagagatgaggtttcgctatgttggccaggcaggtctcaggcttctaacctcaagtaaccctcgggcctcagcctcccaatgtgccgagattacaggtgtgcaccactgtgcctggcaacatgttattttatttaatcctcacatcatccatgagaggtgggttgtactaataccaccatttcataaacaaaaaaactgaggcacagtttaataacttgctggtggtcactcaatagcaaagctggggctcacacacctgcagtccagtcccagtttgtgctcataaccatgtgttatatggcctttccctgcaagttgcaggagagctgcaactttacctgctcttcaccctctgatatggtttggtttggctctgtgtccctactcaaacctcatgtggaattttaatcccctcacgccaggggaggaacctggtcgaaggtgattggatcttgggggtgaatagtgagtgagttctcacttaagtggctatgttttctggggtatataccccaggttcatcctctcatgccgggaaaattcagaacagacacacacagggagtttaggagcacaggttaataggcagaagaaggagaaaggaaaacagccctctctctaatgagagagaggggacttctgagaggaaacaccggcgggtggcgaatgcaccagattttatagtcaggcttgaggaggtggtgtctgatttacatagggctcacagattggttcaatcaggtatgatgttcacatagcgcactgggaaggctggtcaccccaccctaatcttattatgcacatgaattctcctcttggccggcaccagcttgtctgctccttactgtacacttggctggcagagaagggaaaatggagccgccatcttaacgtgtctggttcctagtttctgccggcattcacccgtgcaagctcccagcttgcttgtctatgtgtggggctgcttttcatttaaaagaaaagccttaccaaggactcccgtaccctcactatctacctaagtgatttcttcttaactcctatatcatcataagatctgatggtttaaaagtgtggcacttctcccttctcactgtgtctcctgccaccatgtaagatgtgccttgcttcctcttcaccttctgccatgattgtaagtttcctgaggcctttctggccatgatgaactgtaattcagttaaacctcttttctttataaatcacccagtctcaggtagttctttatagcagtgtgaaaacggactaatacaccctcacagcctcaagaccttgagcagtgtctgatgcttacataatgagtactcctcaaatatttgttcaataaatgcattataagaatctcagttatcctgaggcaagagaaaagaagagggtacaaacttttctttttatttttttgagacagagtttaactcttgtcacccaggctggagtgcagtggtgcgatcttggctcactgcaacctctgctcccaggttcaagcaattttcctgcctcagcctcccaagtagctgggactataggtgcacaccaccacgcccagctaatttttgtatttttagtagagacagggtttcaccatattggccaggctggtctcgaactcctgacctcaggtgatccacctgtttcagcctcccaaagtgctgggattacagggggatacaaatttttcctccttggctatcagttgtaccacttgccacatctcagttttttccacattttgataccagctgctctggttcttccagttttagggtctcttgatcaagatattgtggtttcctctctagaaacaggctccataacattcttcttcttctctctctctctaattaagctattgctgtattagcacttgtaagctacccagtgactgcaggacatgactgcctcaccttctagtctgtgcccaaggtgaaatggatgtggtagtgattcttccacagaccaagtggatccagtgagttgcctataggacatccagaagactcactgacggagtgttgtgggaaagagcagaaagctagaaaccacctctcattggatctttagtccagagaggaatccagcacgcaaaatgcagaaaaatatgatttgtcctcacgtggacatgaagccaaggcattctggagagagtcagggatatggtcagagagcttaggtggccaggaaagagagctctacatgtgtttccacttatggaatcaaggaactcaatacaaagtcccaacagaatcaactcattcctctctattttcccataccccgtcagacccctagtccaagtcaccatcatctcttccctgcatccctgccacagcccctaacctacgccacaaggaactcacccttggcttcctctccagagcacactttcctgacaatgaatcttctaattctagcatcttttacaaaccaggtatcctgagaatttcccaagtcatcatgtctttgttcctttttcttaacagttcctacctcaatttatccctttttctcgggcattttactataaaacagcaagagaaaaccaggctacaccttcaatactttacttggaaagctaagccaaatatccaagttcaaacaaattctgcttcccatataactgtaagacacaattccacagtttcattccactgtatgacaaggatctttttcctccagtttccctaaacatggtcctcatttctttctcagccctcaccaacaacacattatgaaaacctacacttctgccaaccttctacaatgactttgatattctctaaaatgaaacacattttctttaccatgctcttcacttccaagtcctcaccagcagcgcctttaacatccctacttctactaacagtctttctaaggcaagctaggcttttcctatcatgcttctcaaaattcttccacatgctgcccaacacccaattcaaaagccacttccacattgtttaggtatctgttacagtagcacaccatttccaggtaccaaaatctgtattcgtgtactactgctgttgtaacagatgactgcagactttgtggcctgaaattatacaaaattattctttttttctgacgaagtctcacgctgtagcccaggctggagtgcagtggcacaatcttagcttactgcaatctctacctcccgggttcaagtgattctcctgccccagcttcccaagtagttgggtctataggcgtgcaccaccacacctggctaatttttgtatatttagtacatacagggtttcaccatgttggccaggctagtctcaaactcctgacctcaagtgatccacccaccttggcctcccaaagtgctggaattacaggcatgagcccagcctcaaaactacacaaaattattatcttatggttctggaagttccaaatcaaggtgtcagcagagctgcattccttctggaggctctagagtgagaatgttttcttggcttctccagcttctacaagccacctgcattctttggctcatccttatcctcagatcaagtaatggagctctttaaatctcaatctctctctctcacacacacacacacacatacacacacacatacacacacacccctttgcttccactgtggcatctccttctctgattctgcttttgaccctctgcctctctcttataaagacgctactgattatattggacctatttagataatcaaggataatctctccatctcaagatccataacttaatcacaatctgcaaaatcccttttaccatgaaagcttctggggatgaggctatgggcatccttgggggctatgactcagcctaccacattcaccaaccccctattatgggccaagcattggacgacatatggttgcacagtggtaaaaaaactggatatgctctctgctctcaagaaatgtatggtctagtggaagagtcagatatcaatcccaaagtcatacaaataatcaccccaatacaatcattacaagttgagatgagggcactgaaagcctgggaaacatgggagtcccaagggtctctttgaggaagtagcatccaatctgaggttggcagggcagggtgataaggcacccctggatggggaatcagaagaagaccttccaggcagaggcatcagcaagtgcaaaggccctgaagaaaaagagtttgggggatgagggaggtggaaacggcaaaacaagatgaggctgtaaagatgagaaggaccagatcccttgggaacttctggagcatggtggggactttagcttttattcaaaacataacaggaagaggttgaagaggttttttgcttaataaaaaattttttgttggtttttgtttgtttgtttgtttgcttgtttttgagatagggtgtcactctgtagcccaggctggagtgcagtgacgtgatcacagatcacttcagctcctcctgtgctcaagtgatcctcccacctcagcctcccaaagtgctgggattacagtgctggggctgtgagtcaccatgccccaccgggttgaggagtttttatagcaggtgactgatgtgatcactctgctctggagaacaaattgtgtctgggaaacaaatggggatgagaaaaaacaggaacacatatattcagtgaagagaagacagtggcctaaactgggcacatggttgttaaaatagatagcactgcatagatgtaagataggtcttggaccaaaaatgactcataggacttgatggaatttggtgtgtgattcaaaggtctgggatatggccataggggtagctggcatatgaggaatgtagcaaattaatggaaatgagaaattaaagggattaagatgccaggaaggggaatgggccatccacatgcaccttaaaataaatgggaaaacagcacatgggagggaagagaggaggtaagtgctaaaatctacagtggataatggggccagagggactggggtgtcttccaatgatggttagaggaagagagagcacagactcctggtctgagtagcagagagcagatgctatgcaaggagtctcatgtggaaacagtactgggcagggtgaagggcatcagtcccactccagccctggatgagggacagagaaaatgagcagcttccactttaaaggctggcagagaaatgacctcacctaagaccaggaggagcaggaaacccttagggaagataaagtgtagtgcggtaatattggttgcaaaggttattgggggcagatttgagtgggagggtctactgtgggtcgagttaagagagaaaatgcagagcatcaagagagtgaaggtgatggggacaatgtggaaacagtgacaaggggaatttaactgggtgctgggttgccaatgctttaagtcccagtggtgtattccctgggggatgttgggtgcttaggcctctctgtatttaccagtgggctggaagggaaccccagcatgcttttcctcaaggtcttctatgattagggaatgagcaagtgagtctgagcccaggagtatggggttgtgtacaagtgctcttacccccatgttcatcaggtcttctttcgtgattctgcttaaatgtcacctcccaataaagcctttcctaatttttctgacttgtcttttccacatacacagaaccttgtgcagacttctactgtagcacctactatatgcacattattgcctttgctatttatttatttatttatttattcattgagatggagtctcgctctgtcacccaggctggagtacagtggtgcaatctcagctcactgtagcctccacctcccaggttcaagcaattatctgcctcagcctgcccagtagctgggattgcaggtgcatgccaccacacccagctaatttttgtatgtttagtagaaacagggtttcaccatgttggtcaggctagtctcgaactcctggcctcaagtgatctgcctgccttggcctcccaaagtgctgggattacaggcatgagccactgcacccagccacctttgctttttatgtatatctccctaaatagaattgtttctcaagagtgagaataatgcttgaattatttctgttttcctgttactgggtacaaagtctggtagccttggtaggagctacacaaatgtctaattaagtaaaacttagccattgtttgctggtcattattgtactatataaatggtagatttgaagaaaagtttagacactcttcatataatggttactatgaatcattataatgtgtatactatgaagtatacacattacatttaacttgctttcttaatagataattttcaattagttaaattaaatacaacacttcggaaatactgtcaaaaatatgttactggtcttaactaaaatttgcttaaatggccgggtgcagtggctcacgcctgtaatcccaacactttgggaggctgaggcaggtggaccacctgaggtcaggagtttgagatcagcctggccagcatggcaaaactccgtcactactaaaaatacaaaaactagccaagcatggtggcaggcatctgtaatcccagctactcgggaggctgaggagaatcgcttgaacctgggaggctggagattgcagtgagctgagatcacaccacttcactccagtctgggcgacagagcaagactctgtctcaaaaaataataaaactaaaaataataaaatcaaatgtgcataggtaccagtatttcagattttcttgataatgactctggtgtcaagctaatttcattaaaaggcttcaatttttttcattcaatcttgattataataaactcacgtgtcaaaaatcaaatatataaatatatacagtgaaaaggctcacggtaactttctttttatctctacctgagagcttactggttctttcctcaattgttttcttttaatgtcattattttttacgcctaatgactatgcaaatgacttccactctttgtaatcacaaacaatgctgcaataaataatcttgttgataagtcatttcacatgtgtacaagttaatataaggcacttttaattaaactaaatattaacaatgatgttgaatcattaaatctattgcaaactatgcagttacgttaatatggttaaaacaatccctcatttcaagcttcgaagtgttgtccttgaatatcaagtggtagactaaagtactcctgtacatactgttctaagaacattgatcaaaaggagggaaggtgccagttaaccagcaaatacaacattaacacactcagttattaaggggcataaactttattgacatcctacatctatatattcatggaactactgacaatattaataaaatatttgctctaatgttattgaatttttaaaagccagtttagttaatatcatgctttataaccaagtaatggttatatgttatatgattaatgttatatgatttcttttaaataagcataaagaatcatggaaaactatcagcagagccaaccatgaatagttgatgtgtataaccaaaagaaaaaggaaatcagttatcaggagactttcaaaagtgaatatagtaggtgccagaactctccacagggaatttgagaagtcctatttctcgaggcatttaaatggaatagaactgaaagctagaaaaataaactcaaaagaacaatccttcttcagcagaagtaaaagcccaaaatgacctaatagattttttctttcaaaattctaggattcaatgaacactgctctaagtacctcttttgttggatttgtttgtcagggtatgcattcattagtttgcttaaaaactgcattgtttgcaacaaagaaaaacatgttgaacagtattggaattggttaagattcccaaacacatgactttgttctatattctaagaccattccaaggatgtttccctttgcatactgccttaatctgaaaactagtgattgaacaggctggcattactgagtttgttcattacgatttttgtggtgttcagtttatattcctatgtagcatctttttgtatgaattttcctacaatcaatttaaaatcaaccagctatgtgagttttccagtagacatatatagcagctatatgagtttggcaatgggcattaagctacatgagtttgcagctatatgagtctggcaataggcattaatagcagctatatgtgtttggcaataggcattaagaccgtgtaaatactttactgttctctgtgtttttataatcctctgtcttttggaagtcaattaaagctttaacttcaaagaaatccaagtgtggaaagatgtatgtgccattcaaaaacagagaagtataaaacagaatcaattaatctgaatcttgaaagtccagaaataggctctcgatttaactcaaccaatatctgtaaagcaccaacaatggtgcaaggttcctgccatggtagaagaaaaggtaagtgcagaggagttgcagcatatgatcaatatctttttggggcaaggttcatgctacaacagaggaaaaggtaaaaagcacagtcgaattaaagcattgatcagtatcttatttcctgacttcaaaatgtaaatctatgcataccttcccacctttcgcccatgttcattccactgcaaaaacatgatcactgttctcacaaggtgtctggggagtgggctgcatggtgagcaacatggtgcaaattatagataggatggagggagttaaaaaccagtaaatgcaaatataagaacacattttgagatagatcaaatcctctcttatgactagagaaatgcatgcacatagaagaggtgtaggtagattttaaacggtctcatgcactaaactaaactcaggagacaatggagaagccatagattttgcgtggtggaatgacatcgaacagcacataatgaaaattaagcttcagaccagttgggagacttttggaataattcaagcgagaagtaatgagagcataaaccagtatgcagggcaatgggaaagagagaacctatagattcagagaagacagagtccacaggacgtggtacctgcttgaatatgtggaataagagaataagaaagaagtaaaagatggctggctgggcatgtaatcccagtagtttgggaggccaaggcgggcggatcacttgaggtcaagagttctagaccagcctggccaacatggagaaaccccgtctctactaaaaatacaaaaatacaaagccaagcgtggtcgtgggtgtctgtaatcccagctactcaggaggctgaggcaggagaattgcttgaacccgggaggcagaggttgcagtgagccaaaatcacaccactgcactccagcgtgggtgacagaacaagactccgcctcaaaaaaaaaaaaaaaagatggctggacttgggccaaatatacacccttgttacaatcagctgtggctaaggtggagtcacctgcaaagaacatggccatactggccttctcctttgacaggtacaacaggtaggggaagttatctaagtgggaagtaggaaggctaggcaccccaaactcctacactccactaagaaattgcatattaaccttggctaagaaaacacccacacacacttccaaactagttgaagatcgaagaaattagggtgcatatggtatgaaccgagttctattctgtaactatacaccttagatcttgatgggatgaaacagagaagaaagtgataacttatgtgctgtgaacaaacaaaccattggttgtttatctcttgaactaaaagtggagattttcaatattcaaatggcagcgtgaagcttctgcttatatagtttcatatctcagctgactgcaaaggcaagagccaatagccctccactgctctgaataactaaaccatttcagtcttttctacctgtcttccatgctatatgcactctattgtcctggctttaataaccaataatttagatataaaactttgtgtctatatgtgtttgcacattttgcaaaagattggccatacttttcataagatttacaaactggcccagtgcagtgacggatgcctgtaatcccagcactttggaggccaaggtgggtggatcactggagcccaggagttcaagaccagccagtgcaacatggagaaaccccatctctacaaaaaatacaaaaattcaccgggcgtggtggcacatgcctgtggtcccagctactcagggagctgaggcaggaagatcacttgagcccaggaggttgaagctgaagtgagccatgattgtaccactgcactccagcctaggtgacagaatgagaccctatctggaaaaaaaaaaaaaaaaaaaaaaaaatttacaaagtggtgtcagacccaagaaagcctagaacctggagagatcaatagcacaatagactaggagtctgtctactttctcattttgctcttttatttgtattatttatttattcttaagcaattctagtaagttttctgatgctttccttagaagaatgttttcagttaaagtagtttgaggccgggagcggtggctcacgcctgtaatcccagcaccttgggtggccgaggcgggcagatcacgaggtcaggagatcaagaccttcctggctaacatggtgaaaccccgtctctacaaaaaatacaaaaaattagccgggcgtggtggcgggcgcctgtagtcccagctactcaggaagctgaggcaggagaatggcgtgaacccaggaggcggagcttgcagtgagccgagatcgcaccactgtactccagcctgggtgacagagcaagactccgtctcaacaaaagaaaaaaaaaaaagtagtttgcatggcatttagaaaatggaaagcaaaagcaccaattaagcaaggaagtcttgcctcaaacctgcatgtgtttcttctataaacaccagcactattcttataagtgtacagagcactcaccgacactgaaaaccatggaaccctgagtcatgtattatgcttccccatgtagacttcgcagctgtcttttaactctaccttttatcttcaaagttcaaggaaacatttattatgatttcaaattcctgaactcacatctagtcctaaagtcaaaacaatataacttggtatctaaatcaactgaaatgaaatgaaatgcacttggcaaaatcacccttttatgtagtggtggaatgctaacaactgccctgtgaccatcctgtgcaaatccacccttggcctcatcccaggatatacccaaacttcgtctcaactgtgtctctttgtcagtcacgttaagagacagctgtggaatgtttggtctagaatatcccaacaggtgctccttccgtctcaacttctatggagtcaaatagaatttgatctgtaatcctatgtcccaggcttcctaagtcttcctaaagcagatatccatttatagtcaaaacagcaaaaacagttctcacactgagggataaaccgtaaatgcaattgatgaaacattcaaatctacacaccgaaataaagaactgacaaatgaatttgttctttttagattaaagactattcttaatggaactagctgagttttgcaagtatttcacaactttcaataagaaagagccatatgtttttcagtatgacttcagtttattacttgtaatttcctctatttttttctttattggtcttccctgtttccattcttccattttcctgtatttttgggacataactgtctctttcttttagagtttgtttttcccattaattactgaacacaagaaaaatgttttgcaaaatagtctttcccaccaaaaaggtataacagtagaaaaacgtttatgtgacatgctatgctgtattcataaggtaatctaatttccaagctattttaaggatatgagtactcaatttttaggggaaagataatttagctttgtggactttagagcacaaattataagcatagttgaaactgatacctgaacctggtgttccagaaaagacagattctcctttgcgttcaccattgacattgtgagtcactgacaaggtcttatgatgaaactgtttaccaatatttgcaaagtcatgtttatgaataaaagcacctaaatgcttaattttgaaacatttaaaaaatgtgagttgcacctcacatttatttcttggctgaactgcatacaatcccattgcagtggccttagttgaatggacagaggctcaggaaaggccaacagtaggaacatttaccatatgttaggtgaaatgtatagaccagttggacagctgttaaggggataatgttacaatacaaaagctgcctatatttttgacagtaaaatgagcagatcagccatagtttcctcctgtgctcaaaaagaatgatagcacatgcctgtaatcccagcactttgggaggccgagatgggcagatcgcttgaggccaggagttccagaccagcctggccaacatggcaaaactccgtctttacaaaaaaatacgaaaattagctgggcatggtggtaatcccagcttctcaggaggctgaggcacaagaattgcttgaacctaggaggcagaggttgcagtgagtagagatcactctgcggcactccagcctgaacaacacagtgagattctgtcaaaaaaaagaaaagaaaaaagaatgatagcatcatatagaaagtagaggatagctggcacaattatgtctatcatggaaggtcccagattctcactctccagtatagcaaccaaaccttcctgagcataaggctgatcatgccactgggtttccctggggtgggcatctgttatttaaagaaactgtggttaccaacactaaagggaaataaagaaagttctttattttccaacttcctataccagttgagaagaggccatttaaagaagctggcaacctgaaaccccaaaagatgtaactaatacaaaggctcaacatagctgccatttagaaacaatctttcatttatgcacaggaataatttactcataagcccagccctctcagttaggttaataacaccactatcagttttccagctgtttacttccctagctttatttctagacatgagaaataaggaacaaaatatataattaggaaaacaattggaatgtcagcctacggacaaaactgagacaaaatttatataagtagagagtatattgaggtcaaatttgaggactgcaaccaacgaaacaccaaatcaagttgccctgaatatatacagcagttacaagtgggtttttaaaagaaaaaagaggcagtttctaagttgagaagaatttatattaaaataacataaactatataaactattgataggctaaacattgtttttttgtatcacaaattccaggaacataatgagtgaggcagttagtcaggaacaaaatgtctttaaataattactcctgcgcatgcgtacggggggacgtgactgaagtctcacactcctatctctctgggcctgataaattgtgtagccttcacaaagctcagatcgctctgcgctatttttcttttttcagggagttgcacagctactgacagaaagcacatcttggttttagtccatagtacaaaactcaattgtgatcgaatgttttcaacttcctttcataataaggttttaaactcatttcaaataattagatctggttctaatgagctgaaaagcaagagaaattcttgcgtaaaaaaggaaaaatatttttccccttttctctctgtagttctgaaatccagtgaaattactcattactgaaacttatcagatgaaaatattggccagcattaaattcttttcctccccaacccccacctctttttttcctttttctaaccaccacccttttgttaggccagcttaactagctttacatctctgagcctagcctgaggggccacatcctgtgtctggaatgtaaaacctctctccccactaggcctcccgtcttccctccacacacacacacacacacacacacacacacacacacacacacacggacttcccttgcctccaatcccatttacatgtgtatcatttttaaatccccatctaaatttattaagtcataatactcacttaccatttttgtacacatgctactcagtgtatatcctatgtttcttatccaagttgcccattctgcatgtgtctgtcttaaaatacaagctcatgcatgacttaagcccggagaatttgctgaggacaaaggcaatcaagatgccatgtgcaaataacatttttttaaaaaatcaagcttgtgatagcaatacatatttaagagataagtggaaagatgtttgaaatattgtgataaaactaacttaatggaaccttccatacttcttcgatcatacctagaatacaatcatttctttcttaatcttaggaaatatcaggaaacagaaaagagactacttgtagatatcttacctagttccccatgttataaaagtatcttgtggaagggacaggggtttttggacctacaaaaacgcgtaattaaccacaactgcacatggacaggaaaaagctggaaaagcaagactgttcattcttagttttctccttttacttactaaagctataatcaactaaagtgtgtttttcatctgaaatgaaacaagcatgaggaagccagaaatagaacctctaactatagaagaaaagttgtgtgatctctgttgaatacaaaaacgcatagtttccactcggttactgtttaattgtagtgaaagtttttgtttggcaacaaaggcgctatttacatattatttcttctaagtgaaactatggtctgggatgggttgtagataagagcagttgagaaccacgcttcatctccctccttagaaactctgaaacgaggctttattcctaccagaagttcagattgcattatggtcatctcaattccaaaatgttagatggcaagaatatctgcccatccttcactttccttggaaaaagttgctcttcgggttttatatgcgattgcagttttccagtgtgtgaaactaggaaaacaaaacactcaacggtgtacatccctacacctaaatagtcagaaataataggcagctaggctaattatccttgattagcaagatcagagccattagggtgctcactggtttaacaaatgaatgcccttaggcgtctatcatttgtaactcctagaagctttaatttccacaagaaacaaaataagaggggccttctgcttttaacagtgaaaagatcgttctccctcccctctccacccgggtcaactcttccagccgctccctcctgcatcacgaacacacgctgcaggaaagcgcatttacagcccgggacatccccagacctcctctccaaaattccccacctcctgtgcataggagaaactgagagaagccctcacttcctttccaaacttcacaagcaggggagggagctgtagcagactttcacctccgttcccaaaagcgaatgtgaaaaagtccgagaaggcacgtcctgcgagtggaggttaaaccgaaatctgaacagaatgcacggtccccgcaaactacgattgataaagaagatactgagacgtttgcgggggatataagccatggttgtctcgccttcctcccctccctgccaactatgtttcttggagaaatcgccggttcgattcacgcacacatttttgtaaaacacggacaaaaccataagtagttaccttcattgttccgtcggccacgagggaagctcgagctgagcggagggcagatcccaagggtcgtagcccctggccgtgtggaccgggtctgcggctgcagagcgcggtcccggctgcagcaagacctggggcagtgcccgaggcggcggcgagtacacgtggcgggctggattgcagaccggccctctcgcggcggagactcgcgacctagcggattgcatcagcaggaagacactaaggctgctcccccaggccgcccccagatggtggagtctctcccagcccgaagattcggagccagcgcccagacccgagcctcactcactgctcactcccggggtgcagggcagaggtgccagtgttgcaagcaaatgacacggttacccccgaatcagccactgtgggtgcgtatccgagtgtggggatgcccgtgtaacatttatatggagacgtcaaggaggaggaaataaacagatcagaggtcaaatgtgattgccattccgtcatcactggctcctgcccacctccctactgtccccaaagtaactttgctgcatgctgagaggaccacggcacaatcctgcccaaaagtatacatgtatcccccgcggctactttaaatgtacttttgcagtagtcaagaacatgtgcctggtttgccgatctctttcccagagttccttagtcaccctcaaatataaccatttttgccaatttattatgtacatgggggaaaatttttaatcctttaatttgagcatataaaattctttataatgtgaaattgcccagtagcctagttggtcccctgtaagttccctccccacatctatacaaagctccaggattgctacttttccatgtccagcgcctaggtcagcctccatgactgcttaagtgtgctgctggttgacctccgctgcctctctgtcaagctggcaaactcctatttttgtaaatttcaacccaagtctttgctgtggaagttgcttccaatgccctcaaacagaactgggcccttggtctatgtgctgcactttgtccacatcctttctcaaaatcaattattttattttatcattttttaatggagatggggtctcactaaattggccaagctggtcttgaactcctggcctcagcaatcccctcgcctccgcctcccaaattgctgggattacagatgtgagactgtgcccagcttcttaattttactttaattatttgtttgcacagttgtcacttcagtggattatgagattcgtaagggcagatcctttgtcatatttatctcccataccaagtggcctgccgtggaagagagggagtgttgaaaacaaatgtgtcagtgcttaatgactgttggtcaggtgaatgaatggttaaattatgtgttcaataaatgtactgagtgatttctacatgctaggctggtatataacagtggtttgtaatctgaaatgtctcaaaatggagccatttatgacaagggtttcagcctccagtgaaattgctgacccctcaaattgataagcatatgtatggtggactgaggtgataagataacacctgctgtggcctggcaccattggcaagagagtaaagagagaggcagctgagataatggctatgggcacttgcggatgctgtgaaaaccaaaaagtaatgaaaacctgaaagtaactgaccacagccaaaatgcttgcagaagttctgccctgagaaatctgtatctgtaagcatttaatcagccattgagtccttgtgaaacctcctggccccc

SEQ ID NO: 2 - DNA, 569 bp, Artificial Sequence
  BCAT1 cg04543413-cg23792314
cgaagaggtggagattgcaggctgggactccagatttcgggcagggatgcggggaagggaagacgcctcgctggaggcggaatggagggcaaggcgaaggaggatggtgcaggaaacggcgacaaggcgcccggccaggcccgcgagctaccgagacccgggttccaatcctccccccttccgcaaacgcccgggttcgaggtacctggcgggcaagggccgcagcggagcgaagcgggctggccatggggaggctgcggggacgcggggctgcagagagcggcagtggcacggagcgcgcggctggaagcgaaagcaggcggtgtggccaagccccggcgcacggcccatagggcgctgggtaccacgacctggggccgcgcgccagggccaggcgcagggtacgacgcaacccctccagcatcccttggggaggagcctccaaccgtctcgtcccagtctgtctgcagtcgctaaaaccgaagcggttgtccctgtcaccggggtcgcttgcggaggcccgagaatgcgcgccacgaacgagcgcctttccaagcgcagatatttcg

SEQ ID NO: 3 - DNA, 122 bp, Artificial Sequence
  BCAT1 cg20399616
gaagcgggctggccatggggaggctgcggggacgcggggctgcagagagcggcagtggcacggagcgcgcggctggaagcgaaagcaggcggtgtggccaagccccggcgcacggcccatag

SEQ ID NO: 4 - DNA, 1930 bp, Artificial Sequence
  TRIM58 cg26052730-cg09789636
cgactttctggacgacattagagactttaatgtactttatcctgattcagccttccactggatgaagtaacatactctgtcaaatcattttgctcctgtctcagcctcccccatctgacaggtacaccactgagaggctaagcttgaattattcctctgatgagtatctctatagctttgtcatctccaagactggctttgcaacaatcctacaacatgtgtgtattccttgtaggatcctcattgaactaggccagatatttatttaaatatttagaattatttttggctgggctgtaaacctatagttacacacacctatgcaggcatcttcccgtgtcaggcggtgtctcatttctttatcaagaggtaattagtgcaagagaaaaaaatgaagtttgaaaaaaacatttaaaaattgattatgccatataaagggggaaagccttttaagaaacgattatggcatgagaggaccatggagtagctcagtaacaatggaaggggctcggagggtggaaggcgggcgagggatgagagatcacctaatgagaacaatttacgctatcacagcgatgcctgcgctaaagcctagactttatgcagtgtctccatgtaacaaaactgcactggtaccccagaaatctgcactaaatctatataatgatgatggaggctgggcgcggtggctcgcgcctgtaatcccagcactctgggaggtcgaggcgggcagatcacctgaggtcacgagttcaagaccaggctgaccaaaatggtgaaaccccgtctctactaaaaatacaaaaattagccgggcatggtggcgggcgcctgtaatccctgctgcttgcgaggctgaggcaggagaatcgcttgaacccgggaggcggaggctgcagtgagccgagatcgcgccaccgcactccagcctgggcgacagagcgagactccctctcaaaaaaacaaaaacaaaaacaaacaaacaaagatggaaggaaggagggaatttagcaattctccagatctactgccagtgaaaatagcatatgaggagggattccagttagaaatgcccagtttcctgggtgtgtgtgtgacgggggcggggaaccacaacgttaattttatccccgacatagggagcgcctgagtggtggcttttcaccgggtgtggctcgtctgagctcttgaactgaagccagcggacaccacccgtcggcgcctgctttcctggggcgtgggctcctccccctgtgcagaccgcgaggggagacggtgcgggcggccgggagcgcagccctccgggaggcgggtcatggcctgggcgccgcccggggagcggctgcgcgaggatgcgcggtgcccggtgtgcctggatttcctgcaggagccggtcagcgtggactgcggccacagcttctgcctcaggtgcatctccgagttctgcgagaagtcggacggcgcgcagggcggcgtctacgcctgtccgcagtgccggggccccttccggccctcgggctttcgccccaaccggcagctggcgggcctggtggagagcgtgcggcggctggggttgggcgcggggcccggggcgcggcgatgcgcgcggcacggcgaggacctgagccgcttctgcgaggaggacgaggcggcgctgtgctgggtgtgcgacgccggccccgagcacaggacgcaccgcacggcgccgctgcaggaggccgccggcagctaccaggtgaggcgccccccggcgggggctgcgggcgctgcggtgaccgggaagcgggcgacagtccggagcggagccgccgaggccacccgtctcctgagcggctcccacggccgctccccccaccgcgcgccgtcccccccgcccacgcggctcactcagtgtgggtctctttgccttggctgtggtaaccccctttgc

SEQ ID NO: 5 - DNA, 181 bp, Artificial Sequence
  TRIM58 cg20810478-cg07533148
cgtggactgcggccacagcttctgcctcaggtgcatctccgagttctgcgagaagtcggacggcgcgcagggcggcgtctacgcctgtccgcagtgccggggccccttccggccctcgggctttcgccccaaccggcagctggcgggcctggtggagagcgtgcggcggctggggttgggc

SEQ ID NO: 6 - DNA, 62 bp, Artificial Sequence
  TRIM58 cg20810478-cg23054189
cgtggactgcggccacagcttctgcctcaggtgcatctccgagttctgcgagaagtcggacg

SEQ ID NO: 7 - DNA, 122 bp, Artificial Sequence
  TRIM58 cg23054189
cgtggactgcggccacagcttctgcctcaggtgcatctccgagttctgcgagaagtcggacggcgcgcagggcggcgtctacgcctgtccgcagtgccggggccccttccggccctcgggct

SEQ ID NO: 8 - DNA, 122 bp, Artificial Sequence
  TRIM58 cg20810478
gcggctgcgcgaggatgcgcggtgcccggtgtgcctggatttcctgcaggagccggtcagcgtggactgcggccacagcttctgcctcaggtgcatctccgagttctgcgagaagtcggacg

SEQ ID NO: 9 - DNA, 3052 bp, Artificial Sequence
  CDO1 cg07405021-cg16198692
cgctaggcacctatttaatgcaaaaaatactgtagaattgacttctctcccatctcctgtcttcccccaactgccccctcgggactacatccattatgttaatgagctgtacctcagatatctgtgaaaagaccgatcaacccagctggttgcacttcaaaagctttagagtttgtcaatcgagactagttcaaggtccttgaaaatacacttgttctaaagattaaagattaaaaactgttacaacctgtcctggtttaacagtggtttagagtgtaggctctggaacaacaatgctaaccccgggagcctggagccgctatcgatagaagacctagtgcaagtcattcaacttcttcattgcctgccattcctcatatgtgtcaagtgtctggaagagtacctgtcccataacaagtcttcaattacctggagcaaatgcaagaaaatttaaaagctagactctctgctagctttcacggctacagaagggctctaccatgaactaagctgataggagccaaaggaatttcctcaagtcacttgtatatgaaaaatgggatccttagtggtcttcactttttctatttcaaatcctaaaatacagttgaaaccgcccatgccaacaaaactttacgaaacatcccacatgttgaagagtgactaaaagaacctgctcccaagaaaaacaaacttacttctttccttattcttttctttcacttacccctttctcctaaccctaaacccaaaaaaccacaaagactgacgcactaactgctcagaaaaagtagaacctctcacacttcggggtgattaaattttacctctggccccggaggattggtagagagattcgacttaatcaccaagccggggctgcaaaaaagccagcttaccagggcacctcactcaagtacatctgggaagtaggggtctttaatttacattacagatcgtcagaagcaaggataagataaatcagatcatttgggggctgaaaaaaacgacggtcgaatatttaatctgcgaaacttggcagtggatttgaataaacccacagtcggggaagttagttaaattggtttgggaaagggaacagcagaaattccgtagtggcagctgacggccacaagacgctattttacatgagtaaaaatgcttgctataaggtgcccggttgcaggcgaccgaaggcgcagtgatggagaactgacgcgggtgacttgtcttcttcaacatcacacacttctcccgatggctgatgtagtgctcccctgggagcaccctctgctagtccggccagacggttccttaaggtgtcaggagggcccgaacctgagtgcggtctctccgcatctccgcgcccggggttaacgatggcttgggagtgtgggcattgctcccgaggccaggggcaggaggactggtggtttagaagcagaggggtttgcgggagagaagagaacagaaaatctcgcagacgcgcggtggcctcccttcccggcgcctgtccgccgttcccggccggaggtcagcaaggccagtaggatccaccttccgcctgcgctgtggccgcggagccccaagcgagtcgtgccagccccgcggctggccagcgagggggcgagcggcggacgcagcgcggcccgagcttcccgagccagtcactttgggctgcgtcccccacgtccattcctcctcagacggggcgagtgggcgccgcctggcattgaagctgcagcgcgctcacctgtactggtcgaacttggcgtacattgcccactcggtggggtcgctctcgtaggcttccatgatggcctgcacctcctctacattgacctcatcgccggcaaagagctggtgcaggatgcggatcagatcagccagggtccgtggcttcagcacttcggtctgttccatctcgtggggagctggctgcgcgcgcgtctcactgctgggctgcggtggaggagctgagcgagccaaggagctgggggcgagggagcctaacagcccgctagaccgctaagcagacacacacgcacaaacccagcattagagtgccgaaacgtaaggatgtcgtcgcagagacagcaagagacccacccccaggcccctggcagcgcagtggatccgggatcgctggagacgcggtgcacacacaaatcaggttcagatctgtggggttcatcctcccgggccccttttaagcgcttggagtcactaggaatgtaccaacggccctcggagggaggacgaggcggagagccacccaagaaaggtggcggaggcggggagaccctgcgggcacggctcacgcgcacatccccggcttccccgggctccgcgccttcccaagagccccgttgtctccggcgtcccagggatcgcgtgggctccgcgcaatctctcccccactttaacggcgcgttttagccgcccggcctaacgcctctccccgctccacctccgccgctgtggttcgcgacgctgggacgtagacaaaaagggtcggaggagatggctgcggggactgacgctgagtaaaggaggaaaagaaaagggaaggagagggacacttcttccaaatagagaattgtgaggagcctgcgaaaattgaggatggattccactcctaggccagagagtgcctgttttgtcgattgaaactatccacgatatgccccaggctaggtgaaaataagtccaagactttatatatatatatatttaagtatacacacacacatattttttaaaatgcgtactgaaatatattttgacaaaggagactcgggtctctagtaagacaattagtgaaatgatggaaaaccaagaactctttgattttaaaatcaaatattttcttttaaaaaatatccagtgcaaataacctgatatttcatttgatggtgtaaaagggggctttttctgaggggatagggcgggctctgacgcaataatttaagaggctataattatatttgaatctttcaaccttaggcatgtgcttgaaggagcctgctagtacatacatataaggaaacac

SEQ ID NO: 10 - DNA, 83 bp, Artificial Sequence
  CDO1 cg16707405-cg11036833
cggccctcggagggaggacgaggcggagagccacccaagaaaggtggcggaggcggggagaccctgcgggcacggctcacgcg

SEQ ID NO: 11 - DNA, 122 bp, Artificial Sequence
  CDO1 cg11036833
ggcggagagccacccaagaaaggtggcggaggcggggagaccctgcgggcacggctcacgcgcacatccccggcttccccgggctccgcgccttcccaagagccccgttgtctccggcgtcc

SEQ ID NO: 12 - DNA, 1919 bp, Artificial Sequence
  ZNF177 cg05250458-cg14737994
cgtgcatgaatacaggtggaggccaagaccagcattaacaaaggctgtgtgtttgccatggagctcaggagggctaacagtcattttacttctgcctccgaccatcacactttgtccccagaacaaagccaagagtccttggtcaatgaaagtgtgtaaatatcctcacacttcccctagtagttcctttttaggctgtaatcatgtgctaaagaaaagaacctaacttcttaaaatctttccctttttggatgtagattgaaatttttgttttaaacacaatggccctaatgtctaaatctaatgttagtaattaatccctagtgctttaaggatcttctgtttataggcagttattttatggagctggtctctgtgagaggcacagactgccaggaggggttgagatttcctttaaccaggattcttagttctgaatggctctagtgaataacattaacaatgattctctggccgggcactcacgccggtaatcccagcactttgggaggcctagacgggtggatcacttgaggtcaggagttcaagaccagcctggccaacatggtgaaaccccgtctctactaaaaatacaaaaattagccgggcatggtggcagccgcctgtaatcccagctacttggtaggctgaggcaggagaattgcttgaacctgggaggcggaggttgcactgagcccacagtgcgccactgcactacagtctggatgacaagcaaagctcaatctcaaaacaataatttttaaagtaatgattatcttaaccattcttaaatccttctgtctagtaggaatcttattcatgggagtgtctggaaaagggacaaagagcggctattgagatttttttaaagcctggatttgggatgtctgaacagtgaataactccaaagccatttaaaaatcatctttttttctaccaccggactgagcttttcagttttaattccaaggttccaatcatatagatgtgagtcaaaccctgttgggccaaacaggatctcctttgttggcggataatttcaagtgctcacagagaccctccgtttttttttaaatacctggacttaattgtttaaactggagtccggaccaaaacatctctgacctcgggggtcttcctttggtcaggaagaggggagggagcaattggacccctttccctctgcggctgcagagtccaattttcctctggggacatttgctttttcccttggggaagacagtacggattctagttctgtggactcttttacttagctttgagacccaagcaagttaattggcttgtgcctcttattttactctaatgcaatgaataaagacagtcccagccttcgccctaagggagcaggagcacctgcgatgccccgttcccaagtcctcagggcgaatccgccaaatgtgcgagctgggcagcccacccctttcagctgctggccggaagcggaagtgggcgtccgtcgcctcgccatctcccatagctgtcgcctgcagctgagaaagggttgctgtcccagcaggccaggtccaggtgcgccccactagtgaggccggcggaatcgggagggaaggggtcaagggcacagtgcgcagccccggctgctccagaccttgcctgcagcttcccgccccagcctccggctatcgcggcgtttcttctcagaggccgccggctttggttcctcccggcactgctgggcttggggctgccattccgggcattggttcccgggaggaggcaagtgggttcatgtggtcagagtgcgtcggcggggccttttctccactttctgtacccttctctttaaacgtcgtccccagtaggagactattttttctcaagaaaatgctctcttggacctcactttccgttgttagaaggcatatcttgggatgcgc

SEQ ID NO: 13 - DNA, 283 bp, Artificial Sequence
  ZNF177 cg05928342-cg17283453
cgttcccaagtcctcagggcgaatccgccaaatgtgcgagctgggcagcccacccctttcagctgctggccggaagcggaagtgggcgtccgtcgcctcgccatctcccatagctgtcgcctgcagctgagaaagggttgctgtcccagcaggccaggtccaggtgcgccccactagtgaggccggcggaatcgggagggaaggggtcaagggcacagtgcgcagccccggctgctccagaccttgcctgcagcttcccgccccagcctccggctatcgcggc

SEQ ID NO: 14 - DNA, 95 bp, Artificial Sequence
  ZNF177 cg05928342-cg07788092
cgttcccaagtcctcagggcgaatccgccaaatgtgcgagctgggcagcccacccctttcagctgctggccggaagcggaagtgggcgtccgtcg

SEQ ID NO: 15 - DNA, 122 bp, Artificial Sequence
  ZNF177 cg08065231
gccaaatgtgcgagctgggcagcccacccctttcagctgctggccggaagcggaagtgggcgtccgtcgcctcgccatctcccatagctgtcgcctgcagctgagaaagggttgctgtccca

SEQ ID NO: 16 - DNA, 26 bp, Artificial Sequence
  Primer
gaggtttttttttaagggatgttgga

SEQ ID NO: 17 - DNA, 18 bp, Artificial Sequence
  Primer
tccaatcctccccccttc

SEQ ID NO: 18 - DNA, 21 bp, Artificial Sequence
  Primer
aactaaccataaaaaaactac

SEQ ID NO: 19 - DNA, 27 bp, Artificial Sequence
  Primer
tgttyggtgtgtttggattttttgtag

SEQ ID NO: 20 - DNA, 19 bp, Artificial Sequence
  Primer
cacrctctccaccaaaccc

SEQ ID NO: 21 - DNA, 18 bp, Artificial Sequence
  Primer
atagtttttgttttaggt

SEQ ID NO: 22 - DNA, 27 bp, Artificial Sequence
  Primer
aatgtgygagttgggtagtttattttt

SEQ ID NO: 23 - DNA, 26 bp, Artificial Sequence
  Primer
ctactaaaacaacaaccctttctcaa

SEQ ID NO: 24 - DNA, 23 bp, Artificial Sequence
  Primer
agtttattttttttagttgttgg

SEQ ID NO: 25 - DNA, 20 bp, Artificial Sequence
  Primer
gttaaagtgggggagagatt

SEQ ID NO: 26 - DNA, 25 bp, Artificial Sequence
  Primer
tcatcctccccaarcccttttaaac

SEQ ID NO: 27 - DNA, 15 bp, Artificial Sequence
  Primer
gggtttttgggaagg

(19) United States
(12) Patent Application Publication
(10) Pub. No.: US 2019/0203298 A1
(43) Pub. Date: Jul. 4, 2019

(54) METHOD AND KIT FOR THE DIAGNOSIS OF LUNG CANCER

(71) Applicants: INSTITUT D'INVESTIGACIÓ BIOMÈDICA DE BELLVITGE (IDIBELL), Hospitalet De LIobregat (Barcelona) (ES); FUNDACIÓN PARA LA INVESTIGACIÓN DEL HOSPITAL UNIVERSITARIO Y POLITECNICO LA FE DE LA COMUNIDAD, Valencia (ES); FUNDACIÓN PARA LA INVESTIGACIÓN MÉDICA APLICADA, Pamplona (ES)

(72) Inventors: Juan SANDOVAL DEL AMOR, Valencia (ES); Luis MONTUENGA BADIA, Pamplona (ES); Ángel DÍAZ LAGARES, Hospitalet De LIobregat (Barcelona) (ES); Jesús MÉNDEZ GONZÁLEZ, Hospitalet De LIobregat (Barcelona) (ES); David HERVÁS MARÍN, Valencia (ES); Manel ESTELLER BADOSA, Hospitalet De Llobregat (Barcelona) (ES)

(21) Appl. No.: 16/069,309

(22) PCT Filed: Jan. 10, 2017
(86) PCT No.: PCT/EP2017/050380

§ 371 Date: Jul. 11, 2018

(30) Foreign Application Priority Data
Jan. 11, 2016 (EP) 16382007.9

(51) Int. Cl. C12Q 1/6886

(52) U.S. Cl. CPC C12Q 1/6886; C12Q 2600/154; C12Q 2600/172; C12Q 2523/125; C12Q 2600/118; C12Q 2600/156; C12Q 2600/112

(57) ABSTRACT

The present invention refers to the field of cancer and, in particular, to an in vitro method for the diagnosis of lung cancer by determining in a biological sample, taken from a subject, the methylation level of particular genes. It further relates to biomarkers and kits useful for said method.

### FIELD OF THE INVENTION

The present invention refers to the field of cancer and, in particular, to an in vitro method for the diagnosis of lung cancer by determining in a biological sample, taken from a subject, the methylation level of particular genes.

### BACKGROUND OF THE INVENTION

Despite intense research in the field of early cancer detection, there is still a lack of biomarkers for the reliable detection of malignant tumors, including lung cancer, which is the leading cause of cancer-related death worldwide with 1.3 million deaths annually, following data from the World Health Organization (WHO) in 2011. Late diagnosis in lung cancer is one of the main reasons of the extremely high mortality of this disease. On one hand, screening by means of low-dose helical computed tomography (LDCT) has shown to reduce mortality in a large randomized trial, however the positive predictive value is still low. On the other hand, low sensitivity associated with minimally invasive cytologies is also a current hurdle for the accurate diagnosis of lung cancer. Thus, lung cancer early detection using non-invasive strategies is a major challenge to improve survival and its refinement is urgently needed to ameliorate the overall mortality figures for lung cancer worldwide.

Epigenetic biomarkers, mainly DNA methylation, have emerged as one of the most promising approaches to improve cancer diagnosis and present several advantages as compared to other markers, such as gene expression or genetic signatures. DNA methylation alterations are covalent modifications that are remarkably stable and often occur early during carcinogenesis. Additionally, DNA methylation can be detected by a wide range of sensitive and cost-efficient techniques even in samples with low tumor purity. This epigenetic modification can also be detected in different biological fluids which represents a promising tool for non-invasive cancer detection. CpG island hypermethylation of MGMT and GSTP1 has already proven useful for the chemotherapy response prediction in gliomas (Barault L et al., Digital PCR quantification of MGMT methylation refines prediction of clinical benefit from alkylating agents in glioblastoma and metastatic colorectal cancer. Ann Oncol 2015; 26:1994-9) and the screening of prostate cancer (Hogue M O et al. Quantitative methylation-specific polymerase chain reaction gene patterns in urine sediment distinguish prostate cancer patients from control subjects. J Clin Oncol 2005; 23:6569-75), respectively. Great efforts have been undertaken in identifying suitable DNA methylation markers to improve lung cancer diagnosis. However, only one biomarker —SHOX2 methylation—has been commercialized to date (Dietrich D et al, Performance evaluation of the DNA methylation biomarker SHOX2 for the aid in diagnosis of lung cancer based on the analysis of bronchial aspirates. Int J Oncol 2012; 40:825-32), although is not routinely used in the clinic.

Interestingly, the inventors have identified DNA methylation biomarkers already present in early stage lung cancer and globally absent in normal tissue, providing a novel epigenetic tool to improve lung cancer diagnosis.

### SUMMARY OF THE INVENTION

In a first aspect, the present invention refers to an in vitro method for the diagnosis of lung cancer comprising the step of:

a) determining the methylation level of a gene in a test sample, taken from a subject, wherein the gene is one gene, a two-gene combination, a three-gene combination or a four-gene combination, and wherein the gene(s) is(are) selected from the group consisting of BCAT1, TRIM58, ZNF177 and CDO1, and at least one gene is BCAT1;
b) comparing the methylation level determined in step a) to a reference; and
c) identifying the subject as being likely to have lung cancer, if the methylation level of the test sample is higher than the methylation level of the reference, and identifying the subject as unlikely to have lung cancer if the methylation level of the test sample is below the methylation level of the reference.

In second aspect, the present invention refers to a biomarker for in vitro lung cancer diagnosis, wherein the biomarker comprises a methylated gene, containing one or more methylated CpG site(s), wherein the gene is selected from one gene, a two-gene combination, a three-gene combination or a four-gene combination, and wherein the gene(s) is(are) selected from the group consisting of BCAT1, TRIM58, ZNF177 and CDO1, and at least one gene is BCAT1.

In a third aspect, the present invention refers to a kit to carry out the method according to the first aspect of the invention, comprising:
primers for amplifying a CpG-containing nucleic acid of a gene, and/or
means for detecting the presence of methylated CpG site(s) in said amplified nucleic acid,
   wherein the gene is selected from one gene, a two-gene combination, a three-gene combination or a four-gene combination, and
   wherein the gene(s) is(are) selected from the group consisting of BCAT1, TRIM58, ZNF177 and CDO1, and at least one gene is BCAT1.

In a fourth aspect, the present invention refers to the use of a method according to the first aspect of the invention, or of a biomarker according to the second aspect of the invention, or of a kit according to the third aspect of the invention, for in vitro lung cancer diagnosis.

Other objects, features, advantages and aspects of the present application will become apparent to those skilled in the art from the following description and appended claims.

### DESCRIPTION OF THE DRAWINGS

FIG. 1. Epigenetic signature in bronchial aspirates (BAS). (A-D) DNA methylation levels in bronchial aspirates from patients with lung cancer and control donors of Branched Chain Aminoacid Transaminase 1 (BCAT1) gene (A); Tripartite Motif Containing 58 (TRIM58) gene (B); Cysteine Dioxygenase type 1 (CDO1) gene (C), and Zinc Finger Protein 177 (ZNF177) gene (D). NT (light grey circle dots) stands for non-tumoral and T (dark grey square dots) for tumor. P values for all the analyses were calculated using the two-sided Mann-Whitney U test. *** corresponds to p<0.001. (E-H) Receiver Operating Characteristics (ROC) curves and Area Under the Curve (AUC) for BCAT1 gene (E); TRIM58 (F); CDO1 gene (G); ZNF177 gene (H). (I) The AUC for the combination of BCAT1, TRIM58, CDO1 and ZNF177 (referred to as “4-gene epigenetic signature”), using a logistic regression model. (J) Sensitivity (continuous line) and specificity (dotted line) profiles for the different possible cut-off values of the results from the logistic regression model of (I).

FIG. 2.—Results of the epigenetic prediction model for BAS. Nomogram for prediction of cancer risk. To calculate the probability of cancer (POC), a vertical line straight upward from each factor (BCAT1, CDO1, TRIM58, ZNF177) to the Points line had to be drawn. Then, the points from each predictor were summed and with the result, a vertical line was drawn from the Total Points line of the nomogram downwards where the Probability of tumor line was depicted. As a practical example, a patient with the following methylation levels for each gene (BCAT1: 2%, CDO1: 4%; TRIM58: 10% and ZNF177: 15%) would get the corresponding points from the Points line: BCAT11: 8 points, CDO1: 12 points, TRIM58: 12 points and ZNF177: 30 points. The sum of the four values yielded a total points value of 62. These points correspond to a POC higher than 95%.

FIG. 3. Epigenetic signature in bronchioalveolar lavages (BAL). (A-D) DNA methylation levels in bronchioalveolar lavages from patients with lung cancer and control donors of BCAT1 gene (A); TRIM58 gene (B); CDO1 gene (C); ZNF177 gene (D). NT (light grey circle dots) stands for non-tumoral and T (dark grey square dots) for tumor. P values for all the analyses were calculated using the two-sided Mann-Whitney U test. *** corresponds to p<0.001; *p<0.05. (E-H) ROC curves and AUCs for BCAT1 gene (E); TRIM58 (F); CDO1 gene (G); ZNF177 gene (H). (I) The AUC for the combination of BCAT1, TRIM58, CDO1 and ZNF177, using a logistic regression model. (J) Sensitivity (continuous line) and specificity (dotted line) profiles for the different possible cut-off values of the results from the logistic regression model of (I).

FIG. 4. Epigenetic signature in sputums. (A-D) DNA methylation levels in sputums from patients with lung cancer and control donors of BCAT1 gene (A); TRIM58 gene (B); CDO1 gene (C); ZNF177 gene (D). NT (light grey circle dots) stands for non-tumoral and T (dark grey square dots) for tumor. P values for all the analyses were calculated using the two-sided Mann-Whitney U test. *** corresponds to p<0.001; **p<0.01 and *p<0.05. (E-H) ROC curves and AUCs for BCAT1 gene (E); TRIM58 (F); CDO1 gene (G); ZNF177 gene (H). (I) The AUC for the combination of BCAT1, TRIM58, CDO1 and ZNF177, using a logistic regression model. (J) Sensitivity (continuous line) and specificity (dotted line) profiles for the different possible cut-off values of the results from the logistic regression model of (I).

FIG. 5. Epigenetic signature in formalin-fixed paraffin-embedded (FFPE) samples. (A-D) DNA methylation levels of BCAT1 gene (A); TRIM58 gene (B); CDO1 gene (C); and ZNF177 gene (D), in paraffin-embedded sections from patients with lung cancer and control donors. P values for all the analyses were calculated using the two-sided Mann-Whitney U test. NT (light grey circle dots) stands for non-tumoral and T (dark grey square dots) for tumor. *** correspond to p<0.001. (E-H) ROC curves and AUCs with 95% confidence intervals of BCAT1 gene (E); TRIM58 (F); CDO1 gene (G); ZNF177 gene (H).

FIG. 6. Differentially methylated levels in neighboring CpGs on the selected candidate genes. Each data point represents the mean β-value of the group (control: continuous line with empty circles; adenocarcinoma: dotted line and squamous: continuous line) and whiskers show standard error of the mean (s.e.m). Surrounding CpGs are displayed on X axis (significant and selected CpG is highlighted in bold). Empty and crosswise striped squares indicated CpG islands and CpG shores regions respectively. BCAT1 gene (A); CDO1 gene (B); TRIM58 gene (C); ZNF177 gene (D).

FIG. 7.—Expression analysis in lung primary tumor patients using genome-wide DNA methylation datasets. Expression values of BCAT1 gene (A); TRIM58 gene (B); CDO1 gene (C); ZNF177 gene (D), using the TCGA database. P values for all the analyses were calculated using the two-sided Mann-Whitney U test. NT (light grey circle dots) stands for non-tumoral and T (dark grey square dots) for tumor. *** correspond to p<0.001.

FIG. 8.—Expression analysis based on histological subtypes from primary tissues of the TCGA database. Expression values of BCAT1 gene (A); TRIM58 gene (B); CDO1 gene (C); ZNF177 gene (D), in primary tumor samples subclassified by histological subtypes adenocarcinomas (ADC) and squamous cell carcinomas (SCC). Non-Tumour in ADC (light grey circle dots), Tumour in ADC (dark grey circle dots), Non-Tumour in SCC (light grey square dots) and Tumour in SCC (dark grey square dots). P-values for all the analyses were calculated using the two-sided Mann-Whitney U test. *** corresponds to p<0.001, ⋅ corresponds to p<0.1 and n.s. to p>0.1.

### DETAILED DESCRIPTION OF THE INVENTION

It must be noted that as used in the present application, the singular forms “a”, “an” and “the” include their correspondent plurals unless the context clearly dictates otherwise. Unless defined otherwise, all technical and scientific terms used herein have the same meaning as commonly understood by one of ordinary skill in the art to which this invention belongs.

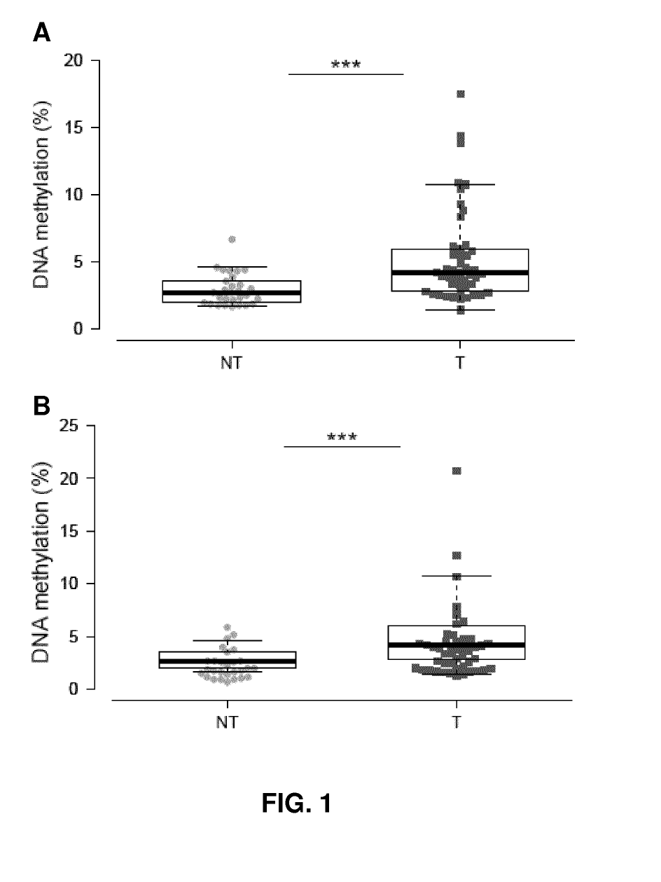

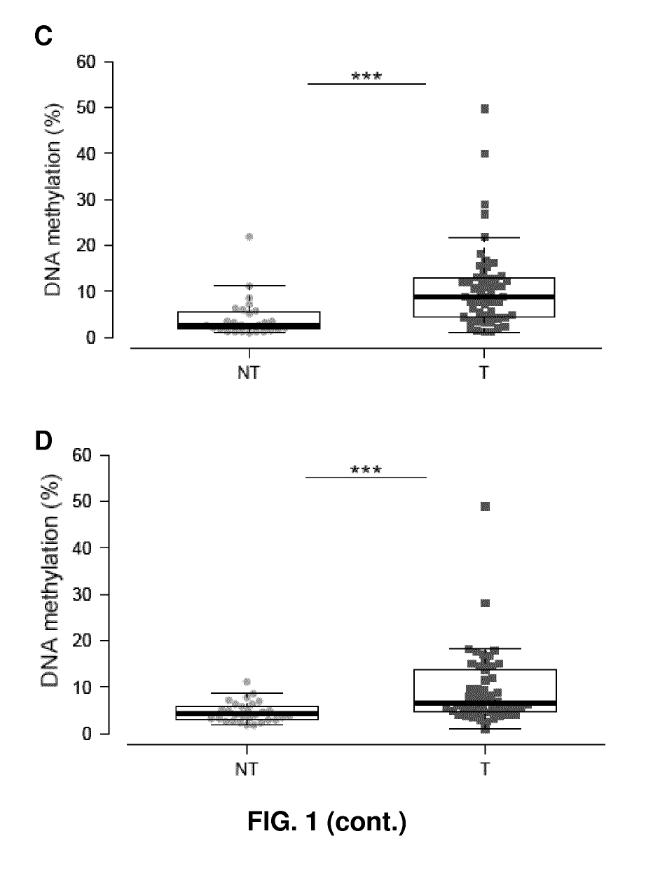

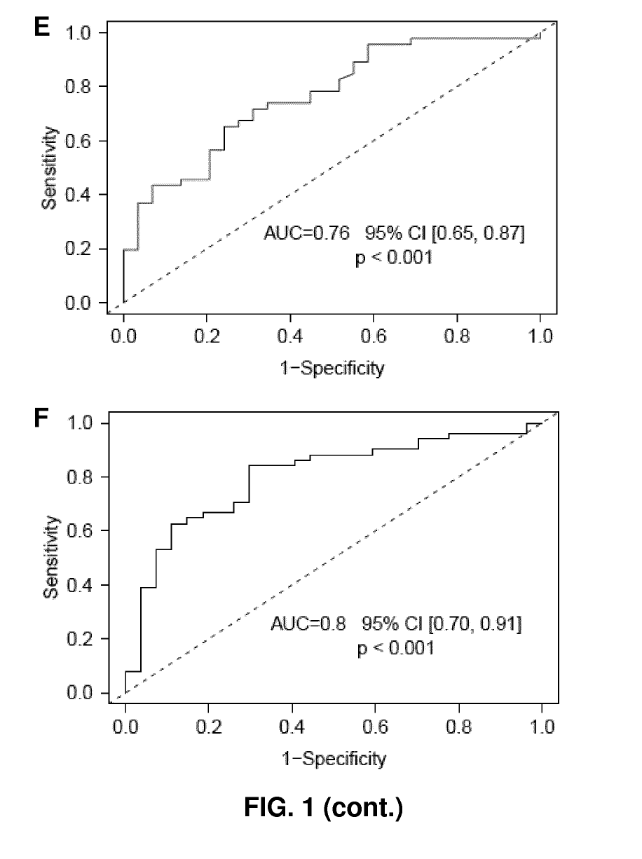

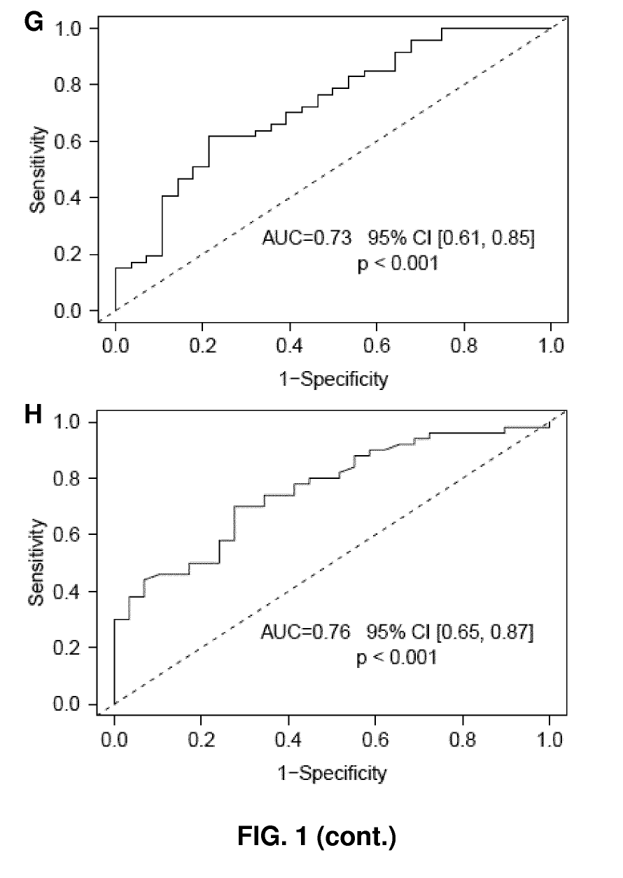

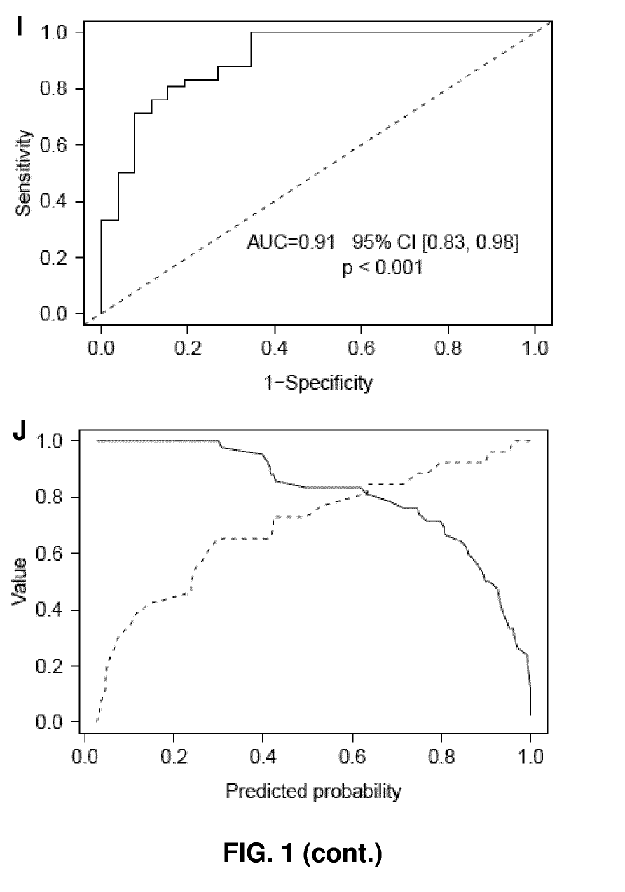

The authors of the present invention have found that four genes, BCAT1, TRIM58, CDO1 and ZNF177, are differentially methylated in lung cancer. As shown in the Examples, the level of methylation of any one of the genes BCAT1, TRIM58, ZNF177 and CDO1 was higher in the samples taken from subjects with lung cancer than in control samples (samples taken from tumor-free subjects) (see panels A-D of FIGS. 1, 3-5). Thus, in a first aspect, the present invention refers to an in vitro method for the diagnosis of lung cancer (referred to as method of the invention) comprising the steps of:

a) determining the methylation level of a gene in a test sample, taken from a subject, wherein the gene is one gene, a two-gene combination, a three-gene combination or a four-gene combination, and wherein the gene(s) is(are) selected from the group consisting of BCAT1, TRIM58, ZNF177 and CDO1;
b) comparing the methylation level determined in step a) to a reference; and
c) identifying the subject as being likely to have lung cancer, if the methylation level of the test sample is higher than the methylation level of the reference, and identifying the subject as unlikely to have lung cancer if the methylation level of the test sample is below the methylation level of the reference.

The present invention may be practiced using each gene separately or using combinations of two, three or four genes. Thus, any of the genes identified in the present application may be used individually or as a set of genes in any combination with any of the other genes that are recited in the application, i.e. a two-gene combination, a three-gene combination or a four-gene combination, wherein the genes are selected from the group consisting of BCAT1, TRIM58, ZNF177 and CDO1. In the context of the present invention the term “diagnosis” refers to determining the likelihood of having or suffering from lung cancer. It also refers to identifying or determining the presence of lung cancer.

The term “lung cancer” refers to non-small cell lung cancer (NSCLC) and small cell lung cancer (SCLC). NSCLC accounts for about 80% of the lung cancers and is a heterogeneous clinical entity with major histological subtypes such as squamous cell carcinoma (SCC), adenocarcinoma (ADC) and large cell carcinoma. According to the histological classification of the WHO/International Association for the Study of Lung Cancer (Travis et al., Histological typing of lung and pleural tumours, 3^{rd} ed. Berlin: Springer-Verlag, 1999) other subtypes of NSCLC are large cell carcinoma; adenosquamous carcinoma; carcinoma with pleomorphic, sarcomatoid or sarcomatous elements; carcinoid tumour; carcinoma of salivary gland type and unclassified carcinomas. Preferably, in the present invention the lung cancer is a NSCLC, more preferably NSCLC is of the subtype ADC, SCC or large cell carcinoma, and even more preferably ADC or SCC.

A common feature of the different subtypes of NSCLC is the somewhat slower growth and spread compared to SCLC, enabling surgical eradiaction is its early stages. Disappointingly, only a minor fraction of NSCLC cases are currently diagnozed in clinical stages I to II, where surgical removal is the therapy of choice. The major reasons for late diagnosis are the late appearance of symptoms and, as mentioned above, a lack of reliable biomarkers for its early detection. Interestingly, the present invention provides methods that allow diagnosis of lung cancer in early stage. Thus, preferably, the methods of the first aspect of the present invention refers to method for detecting lung cancer in early stage and more preferably NSCLC in early stage. In the context of the present invention, lung cancer in early stage refers to stage 0, stage I and stage II, i.e. NSCLC in stage 0, I and II and SCLC in stage 0, I and II; and more preferably it refers to stage I. The classification of the different stages of lung cancer and its (sub)types is well known by the skilled in the art and in the present invention the classification according to AJCC Cancer Staging Manual 7th edition; Chapter 25; Lung—original pages 253-266 is used.

In the context of the present invention the term “subject” refers to any member of the class Mammalia, including, without limitation, humans and non-human primates such as chimpazees and other apes and monkey species; farm animal such as cattle, sheep, pigs, goats and horses; domestic mammals such as dogs and cats; laboratory animals including rodents such as mice, rats and guinea pigs, and the like. The term does no denote a particular age or sex. Thus, adult and newborn subjects, as well as fetuses, whether male or female, are intended to be included within the scope of this term. The subject is preferably a human.

The term “test sample”, as used herein, refers to a biological sample taken from a subject under study. The biological sample contains any biological material suitable for detecting the desired methylation level in one or more CpG site(s) and is a material comprising genetic material from the subject. In the present invention, the sample comprises genetic material, e.g., DNA, genomic DNA (gDNA), complementary DNA (cDNA), RNA, heterogeneous nuclear RNA (hnRNA), mRNA, etc., from the subject under study. In a particular embodiment, the genetic material is DNA. In a preferred embodiment the DNA is genomic DNA. In another preferred embodiment, the DNA is circulating DNA. Isolating the nucleic acid of the sample can be performed by standard methods known by the person skilled in the art, such as those described in Sambrook et al., (Molecular Cloning: A Laboratory Manual, New York: Cold Spring Harbor Press, 1989).

Methylated genes are expressed in tumor tissue samples and they can also be detected in biological fluids comprising tumor cells. Therefore, in a particular embodiment, the biological sample is a lung tissue sample or a biological fluid, and in yet another more particular embodiment the biological sample is a biological fluid, so the method is less invasive than the ones requiring a tissue sample taken by means of biopsy. In a preferred embodiment of any of the methods of the present invention defined above, the biological sample is a biological fluid selected from BAS, BAL, sputum, saliva, whole blood, serum, plasma, urine, feces, ejaculate, a buccal or buccal-pharyngeal swab, pleural fluid, peritoneal fluid, pericardic fluid, cerebrospinal fluid and intra-articular fluid. Preferably the biological fluid is selected from BAS, BAL, blood and sputum, more preferably BAS, BAL and blood, and even more preferably the biological sample is BAS.

The term “gene” is intended to include not only regions encoding gene products but also regulatory regions including, e.g., promoters, termination regions, translational regulatory sequences (such as ribosome binding sites and internal ribosome entry sites), enhancers, silencers, insulators, boundary elements, replication origins, matrix attachment sites, and locus control regions. The term “gene” further includes all introns and other DNA sequences spliced from the mRNA transcript, along with variants resulting from alternative splice sites. The term “gene” further includes any portion of a gene, e.g. any portion of the regions mentioned above. The gene(s) or gene portion(s) of the invention are also referred herein as marker(s) or marker gene(s).

The genes according to the invention include:
BCAT1, which refers to the gene Branched Chain Amino-Acid Transaminase 1. BCAT1 is a cytosolic enzyme that promotes cell proliferation though aminoacid catabolism and high frequency of methylation on BCAT1 promoter in colorectal cancer has been reported. Its sequence reference is GenBank NM_005504.
TRIM58, which refers to the gene tripartite motif containing 58. TRIM58 is an E3 ubiquitin ligase superfamily member that has been shown methylated in hepatocytes derived from hepatitis B virus-related hepatocellular carcinoma. Its sequence reference is GenBank NM_015431.
CDO1, which refers to the gene cysteine dioxygenase type 1. CDO1 has been postulated as a tumor suppressor gene silenced by promoter methylation in multiple human cancers, including breast, esophagus, lung, bladder and stomach. Its sequence reference is GenBank NM_001801.
ZNF177, which refers to the gene of a zinc finger transcription factor that has been reported to be methylation-silenced in gastric cancer cell lines. Its sequence reference is GenBank NM_003451.

For each of the genes mentioned above, the inventors have identified portions of said genes that are particularly useful in the method of the present invention. Thus, in a particular embodiment of the present invention, BCAT1 gene refers to a portion of BCAT1 gene comprising a sequence selected from the group consisting of SEQ ID NOs 1-3. In another particular embodiment, TRIM58 gene refers to a portion of TRIM58 gene comprising a sequence selected from the group consisting of SEQ ID NOs 4-8. In another particular embodiment, CDO1 gene refers to a portion of CDO1 gene comprising a sequence selected from the group consisting of SEQ ID NOs 9-11. In another particular embodiment, ZNF177 gene refers to a portion of ZNF177 gene comprising a sequence selected from the group consisting of SEQ ID NOs 12-15. In a preferred embodiment of any of the embodiments of this paragraph each gene is represented by any one of the mentioned sequences. Variants according to the present invention include nucleotide sequences that are at least 60%, 65%, 70%, 75%, 80%, 85%, 90%, or 95% similar or identical to any one of sequences SEQ ID NO 1-15. The degree of identity between two nucleic acid molecules is determined using computer algorithms and methods that are widely known for the persons skilled in the art. The identity between two nucleic acid sequences is preferably determined by using the BLASTN algorithm (BLAST Manual, Altschul et al., 1990, NCBI NLM NIH Bethesda, Md. 20894, Altschul, S., et al., J. Mol Biol 215:403-10).

The term “methylation” or “DNA methylation”, as used herein, refers to a biochemical process involving the addition of a methyl group to the cytosine (C) or adenine (A) DNA nucleotides, preferably to cytosine. DNA methylation at the 5 position of cytosine, resulting in 5-methylcytosine (5-mC), may have the specific effect of reducing gene expression and has been found in every vertebrate examined. In adult non-gamete cells, DNA methylation typically occurs in a CpG site. The term “CpG site”, as used herein, refers to regions of DNA where a cytosine nucleotide occurs next to a guanine (G) nucleotide in the linear sequence of bases along its length. “CpG” is shorthand for “C-phosphate-G”, that is, cytosine and guanine separated by only one phosphate; phosphate links any two nucleosides together in DNA. The terms “CpG” and “CpG site” may be used interchangeably in the present context.

The methylation level of a gene can be determined at one or more CpG site(s). If more than one CpG sites are used, methylation can be determined at each site separately or as an average of the CpG sites taken together. Preferably, the methylation of more than one CpG site is determined and the methylation level is given as an average value of the CpG sites, in particular in the form of average beta-value or percentage. The techniques for detection of DNA methylation are known in the art and include, without limitation, bisulfite modification based technologies, enzymatic digestions based methodologies, affinity-enriched based technologies and high throughput analysis. These techniques include bisulfite sequencing, Methylation Specific PCR (MSP), pyrosequencing, ConLight-MSP (Conversion-specific Detection of DNA Methylation Using Real-time Polymerase Chain Reaction), SMART_MSP (Sensitive Melting Analysis after Real Time-Methylation Specific PCR), Matrix-assisted laser desorption/ionization-time of flight (Mass Array Epityper Sequenom), HPLC (High performance liquid chromatography), Methyl-Beaming, droplet digital PCR, COBRA (Combined Bisulfite Restriction Analysis), reduced representation bisulfite sequencing (RRBS), HELP assay (Hpall tiny fragment Enrichment by Ligation-mediated PCR) and MethDet (methylation detection), Methylated DNA immunoprecipitation (MeDIP), Methyl-Cap, methylation binding domain assays, arrays and Whole Genome Bisulfite Sequencing.

Preferably the detection of methylation in any one of the methods described in the present invention is performed by bisulfite sequencing or pyrosequencing. More preferably the level of methylation is determined by pyrosequecing since pyrosequencing is an affordable and quantitative method that counterbalances some weaknesses of previous and extensively used methods, due to its easy standardization and lower false positive rate. Moreover, pyrosequencing is a suitable approach in a clinical setting because it represents a quantitative and reproducible method able to detect multiple CpGs not only in FFPE tissues but also in non-invasive samples as biological fluids, as shown in the Examples of the present application. Methods for pyrosequencing are well known in the art and described, for example, in Nyrén, P. (The History of Pyrosequencing. 2007. Methods Mol Biology 373: 1-14). Thus, in a preferred embodiment of any one of the embodiments of the first aspect of the invention, the methylation level is determined by pyrosequencing.

Bisulfite sequencing method for detecting a methylated CpG-containing nucleic acid comprises the steps of: bringing a nucleic acid-containing sample into contact with an agent that modifies unmethylated cytosine; and amplifying the CpG containing nucleic acid in the sample using CpG-specific oligonucleotide primers, wherein the oligonucleotide primers distinguish between modified non-methylated nucleic acid and methylated nucleic acid and detect the methylated nucleic acid. The amplification step is optional and desirable, but not essential. The method relies on the PCR reaction to distinguish between modified (e.g., chemically modified) unmethylated DNA and methylated DNA. Such methods are described in U.S. Pat. No. 5,786,146 relating to bisulfite sequencing for detection of methylated nucleic acid.

The pyrosequencing method is a quantitative real-time sequencing method modified from the bisulfite sequencing method. Similarly to bisulfite sequencing, genomic DNA is converted by bisulfite treatment, and then, PCR primers corresponding to a region containing no CpG base sequence are constructed. Specifically, the genomic DNA is treated with bisulfite, amplified using the PCR primers, and then subjected to real-time base sequence analysis using a sequencing primer. The level of methylation is expressed as percentage or beta-value.

In the context of the present invention the term “reference” or “reference level” refers to a value or level, which has been determined by measuring the methylation level of the same gene(s) as the test sample in a biological sample taken from a subject or a population of subjects not suffering from lung cancer, i.e. lung cancer-free (also referred to as non-tumoral) subject/population. The sample taken from a lung cancer-free subject is also referred as “control sample”, thus reference also refers to methylation level of a control sample. Preferably, the control sample is a sample of subjects matched on age and body mass index to the subject analysed. Preferably, the reference is a reference value, a cut-off value or a threshold.

As mentioned above, the method of the invention may comprise determining the methylation level of a combination of genes selected from the group consisting of BCAT1, TRIM58, CDO1 and ZNF177 (two-gene, three-gene or four-gene combination), in which case the methylation level is compared to a combined reference-level of said combination of genes. The measured methylation levels can be combined by arithmetic operations such as addition, subtraction, multiplication and arithmetic manipulations of percentages, square root, exponentiation, and logarithmic functions. Levels can also be combined following manipulation using various models e.g. logistic regression and maximum likelihood estimates. Various means of calculating the combined reference-value can be performed by means known to the skilled in the art.

In a particular embodiment of the method of the invention according to any one of the embodiments mentioned above, the level of methylation of the test sample is higher than the level of methylation of the control sample, when it is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% or more higher than in the control sample. Preferably it is at least 50% higher.

In a particular embodiment of the method of the invention, the methylation level of the test sample is considered to be higher than the reference when the differences in average beta-values between groups (tumoral and non-tumoral) is higher than a set threshold, preferably higher than 0.20.

The first aspect of the invention also refers to an in vitro method for the diagnosis of lung cancer comprising the steps of:

a) determining the methylation level of a gene in a test sample, taken from a subject, wherein the gene is one gene, a two-gene combination, a three-gene combination or a four-gene combination, and wherein the gene(s) is(are) selected from the group consisting of BCAT1, TRIM58, ZNF177 and CDO1;
b) constructing a percentile plot of the methylation level of said gene or combination of genes obtained from a sample from a non-tumoral population;
c) constructing a ROC curve based on the methylation level determined in the non-tumoral population and on the methylation level determined in a population with lung cancer;
d) selecting from the ROC-curve the desired combination of sensitivity and specificity;
e) determining from the percentile plot the methylation level corresponding to the determined or chosen specificity; and
f) predicting that the subject is likely to have lung cancer, if the methylation level of said gene or combination of genes in the test sample is equal to or higher than said methylation level corresponding to the desired combination of sensitivity/specificity, and predicting that the subject is unlikely to have lung cancer, if the methylation level in the test sample is lower than said methylation level corresponding to the desired combination of sensitivity/specificity.

The sensitivity of any given screening test is the proportion of individuals with the condition who are correctly identified or diagnosed by the test, e.g. the sensitivity is 100%, if all individuals with a given condition have a positive test. The specificity of a given screening test is the proportion of individuals without the condition who are correctly identified or diagnosed by the test, e.g. the specificity is 100%, if all individuals without the condition have a negative test result. Thus, the sensitivity is defined as the (number of true-positive test results)/(number of true-positive+number of false-negative test results). The specificity is defined as (number of true-negative results)/(number of true-negative+number of false-positive results). The specificity of the method according to the invention is preferably from 70% to 100%, such as from 75% to 100%, more preferably 80% to 100%, more preferably 90% to 100%. Thus, in one embodiment of the present invention the specificity is 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The sensitivity of the method according to the invention is preferably from 70% to 100%, such as from 75% to 100%, more preferably 80% to 100%, more preferably 90% to 100%. Thus, in one embodiment of the present invention the sensitivity is 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In another embodiment of the first aspect of the invention according to any one of the embodiments described above, when the method for the diagnosis refers to a method for determining the likelihood of having or suffering from lung cancer, the method of the invention comprises the use of an algorithm to calculate the likelihood of having lung cancer or the probability of cancer (POC).

The inventors generated a mathematical algorithm to calculate the probability of cancer for any sample type and gene combination. The general form of said algorithm was (formula I):

$$Pr ( Cancer ) = \frac{{e}^{a + b * TRIM 58 + c * ZNF 177 + d * CDO 1 + e * BCAT 1}}{1 + {e}^{a + b * TRIM 58 + c * ZNF 177 + d * CDO 1 + e * BCAT 1}} * 100$$

where the coefficients a, b, c, d and e take the log-odd values of cancer estimated by a multivariable logistic regression model adjusted using maximum likelihood to methylation values data from a specific sample type and a specific combination of the four genes.

In a particular embodiment, the method of the invention comprises determining the methylation level of BCAT1 gene and the algorithm used to calculate the POC is of formula II:

$${Pr ( Cancer )}_{BAS} = \frac{{e}^{- 1.86 + 0.63 * BCAT 1}}{1 + {e}^{- 1.86 + 0.63 * BCAT 1}} * 100$$

In another embodiment, the method comprises determining the methylation level of BCAT1 and TRIM58 genes and the algorithm is of formula III:

$${Pr ( Cancer )}_{BAS} = \frac{{e}^{- 3.03 + 0.24 * TRIM 58 + 0.55 * BCAT 1}}{1 + {e}^{- 3.03 + 0.24 * TRIM 58 + 0.55 * BCAT 1}} * 100$$

In another embodiment, the method comprises determining the methylation level of BCAT1, TRIM58 and ZNF177 genes and the algorithm is of formula IV:

$${Pr ( Cancer )}_{BAS} = \frac{{e}^{- 5.15 + 0.20 * TRIM 58 + 0.32 * ZNF 177 + 0.66 * BCAT 1}}{1 + {e}^{- 5.15 + 0.20 * TRIM 58 + 0.32 * ZNF 177 + 0.66 * BCAT 1}} * 100$$

In another embodiment, the method comprises determining the methylation level of BCAT1, TRIM58, ZNF177 and CDO1 genes and the algorithm is of formula V:

$${Pr ( Cancer )}_{BAS} = \frac{{e}^{- 6.13 + 0.18 * TRIM 58 + 0.30 * ZNF 177 + 0.47 * CDO 1 + 0.59 * BCAT 1}}{1 + {e}^{- 6.13 + 0.18 * TRIM 58 + 0.30 * ZNF 177 + 0.47 * CDO 1 + 0.59 * BCAT 1}} * 100$$

In a preferred embodiment according to any of the embodiments of the six previous paragraphs, the sample taken from the subject, of which the methylation level is determined, is a BAS. More preferably, the methylation level is determined by pyrosequencing and using the primers depicted in Table 7 (below).

In a particular embodiment of the invention according to any one of the preceding seven paragraphs, the likelihood of having lung cancer or the POC is at least 50%, at least 60%, at least 70%, at least 80%, at least 85%, at least 90%, at least 95%, or 100%, preferably it is between 80% and 100%, and more preferably, between 90% and 100%.

As mentioned above, the method of the invention may comprise determining the methylation level of one gene or of a combination of genes (two-gene, three-gene or four-gene combination) selected from the group consisting of BCAT1, TRIM58, ZNF177 and CDO1. Thus, in a particular embodiment of any of the methods according to the first aspect of the invention described above, the methylation level of at least BCAT1 or at least TRIM58 or at least CDO1 or at least ZNF177 is determined. In a particular embodiment methylation of BCAT1 is determined, in another particular embodiment methylation of TRIM58 is determined, in another particular embodiment methylation of ZNF177 is determined, and in another particular embodiment methylation of CDO1 is determined. As shown in FIGS. 1, 3-5 the level of methylation (panels A-D) and the AUC (panels E-H) of any of these genes was higher in the test samples than in control samples. In a preferred embodiment, the methylation of the gene BCAT1 or TRIM58 is determined, since any one of these genes provides the highest AUC (see Table 9, in Example 2) and thus the highest accuracy for a diagnostic method determining only the methylation level of one gene. Interestingly, by combination of the different marker genes according to the invention a synergistic effect is achieved (see Table 9). Specifically as used herein synergy refers to the phenomenon in which several markers acting together created a “gene combination” with greater sensitivity or specificity for diagnosis, than that predicted by knowing only the separate genes sensitivity or specificity.

Thus, in a preferred embodiment of the method of the first aspect of the invention, according to any one of the embodiments mentioned above, the methylation level is determined in a combination of two genes (two-gene combination) selected from the group consisting of BCAT1, TRIM58, CDO1 and ZNF177. In a preferred embodiment, the methylation level is determined in two genes selected from the group consisting of BCAT1, TRIM58, CDO1 and ZNF177, wherein one of said genes is BCAT1. In another embodiment, the methylation level is determined in two genes selected from the group consisting of BCAT1, TRIM58, CDO1 and ZNF177, wherein one of said genes is TRIM58. In another embodiment, the methylation level is determined in two genes selected from the group consisting of BCAT1, TRIM58, CDO1 and ZNF177, wherein one of said genes is ZNF177. In another embodiment, the methylation level is determined in two genes selected from the group consisting of BCAT1, TRIM58, CDO1 and ZNF177, wherein one of said genes is CDO1. In a preferred embodiment according to the previous embodiments, methylation of a two-gene combination is determined and the two-gene combination is selected from BCAT1 and TRIM58; BCAT1 and ZNF177; BCAT1 and CDO1; TRIM58 and ZNF177; TRIM58 and CDO1; or ZNF177 and CDO1. Preferably, the methylation of any two-gene combination in which one of the genes is BCAT1 is determined, i.e. BCAT1 and TRIM58; BCAT1 and ZNF177; BCAT1 and CDO1. As shown in Table 9, these BCAT1 containing two-gene combinations have higher AUC in all the different biological samples indicating that the combination improves specificity and sensitivity leading to a higher prediction efficacy.

In another embodiment of the method of the first aspect of the invention according to any one of the embodiments described above, the methylation level is determined in a combination of three genes (three-gene combination) selected from the group consisting of BCAT1, TRIM58, CDO1 and ZNF177. In a particular embodiment, one of the three genes is BCAT1, in another embodiment, one of the three genes is TRIM58, in another particular embodiment, one of the three genes is ZNF177, in another embodiment, one of the three genes is CDO1. In a preferred embodiment according to any of the previous embodiments, it is detected the methylation of a three-gene combination selected from BCAT1, TRIM58 and ZNF177; BCAT1, TRIM58 and CDO1; BCAT1, ZNF177 and CDO1; or TRIM58, ZNF177 and CDO1. Preferably, the methylation of any one of the three-gene combination in which one of the genes is BCAT1 is determined, i.e. BCAT1, TRIM58 and ZNF177; BCAT1, TRIM58 and CDO1; BCAT1, ZNF177 and CDO1. As shown in Table 9, these BCAT1 containing three-gene combinations have higher AUC in all the different biological samples. The combination improves specificity and sensitivity leading to a maximized prediction efficacy, identifying cancer cases that would not be detected with two-gene combinations.

In a further embodiment of the first aspect of the invention according to any one of the embodiments described above, the methylation of a four-gene combination is determined, wherein the four-gene combination is BCAT1, TRIM58, CDO1 and ZNF177. As shown in FIGS. 1, 3-5 and in Table 9, the AUC of the combination of these four genes was equal or higher than 0.85, in particular 0.91 for BAS samples, 0.85 for BAL samples, and 0.93 for sputum. Interestingly, internal validation of the AUC estimate for this combination yielded optimism corrected AUC of 0.90, showing high generalization of the predictive capacity of the combination.

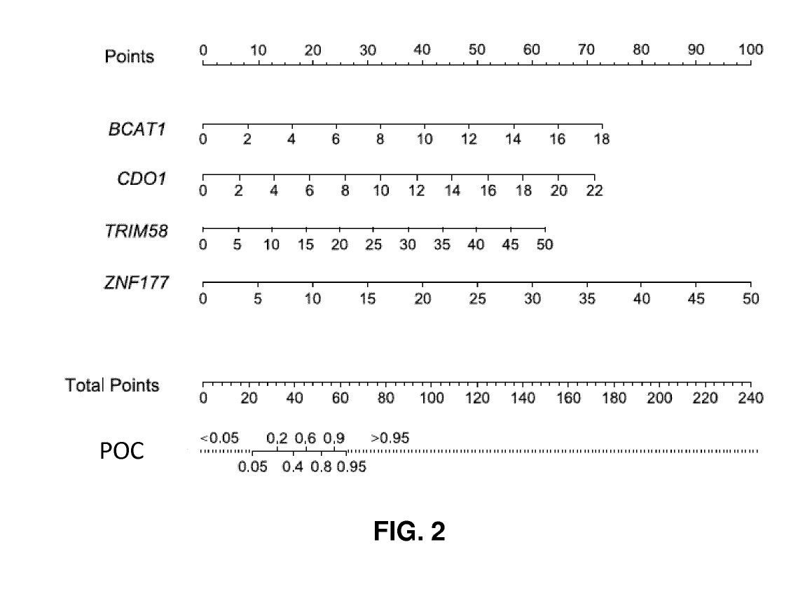

Moreover, a nomogram based on the results of this four-gene combination is provided as a predictive tool for clinical diagnostic use (Example 2, FIG. 2). The use of this nomogram for in vitro lung cancer diagnosis is within the scope of the present invention. The nomogram has been obtained using the algorithm of formula (V). Results of the nomogram provide an individual probability (0%-100%) for suffering lung cancer for each patient (FIG. 2). Evaluation of the full range of predictions of the model shows that shifting the cut-off to POC=30% would yield a sensitivity of 100% and a specificity of 65.4% and shifting the cut-off to POC=80% would yield a sensitivity of 71.4% and a specificity of 92.3%. Sensitivity and specificity at the optimal cut-off (POC=63%) were 84.6% and 81.0% respectively. Thus, it has been shown by the inventors that this embodiment of the four-gene combination allows a particularly accurate and reliable diagnosis of lung cancer.

Interestingly, the methods of the invention described above allow an early diagnosis mainly based in non-invasive or minimally invasive samples. The performance of the methods in these type of samples, such as BAS, BAL and sputum, was outstanding despite the limited number of tumoral cells compared to FFPE samples. Surprisingly, the methods of the present invention provide a balanced and flexible approach able to cater to both extreme scenarios: the high sensitivity and low specificity of LDCT in screening programs and the high specificity and low sensitivity of cytology in respiratory specimens routinely used for lung cancer diagnosis. The epigenetic signatures of the present invention improves the predictions of cytology by providing a method for continuous predictions, in particular the four-gene epigenetic signature (See Example 2). In the context of the present invention, any one of the genes or the two-, three- and four-gene combinations described herein are also referred to as “epigenetic signatures of the invention” or as “one-gene epigenetic signature”, “two-gene epigenetic signature”, “three-gene epigenetic signature” or “four-gene epigenetic signature”, respectively. Cytology is a useful dichotomized classifier producing two types of predictions: 100% positive or 0% positive (100% negative). Therefore, the final output will be either a complete success or a total failure. In contrast, the epigenetic signatures of the present invention based in a logistic regression model, represented by a nomogram, are able to produce a continuous range of predictions between 100% positive and 0% positive. This way, not all predictions are a complete success or a total failure, uncertainty can be measured for each prediction and errors are almost always lower. In a virtual situation where the method of the invention predicts two negative samples with different probability of being positive: such as 5% and 49%, the bimodal classifier predictor (cytology) would have output only absolute responses: negative and negative. Therefore, no information about uncertainty and chances of being positive for patient 1 (very low) and patient 2 (almost 50%) would have been delivered. Thus, the four-gene epigenetic signature achieved higher diagnostic efficacy in bronchial fluids as compared with conventional cytology for early lung cancer detection. It also yielded a notably high specificity, one of the Achilles heels of LDCT and other methylation genes, and also improved sensitivity, which is generally limited when using cytology for early lung cancer diagnosis.

In a particular embodiment of any one of the methods of the first aspect of the invention described above, the methylation level of one or more of the genes BCAT1, TRIM58, CDO1 and ZNF177 is determined at one or more CpG site(s). In a particular embodiment, the CpG site(s) is/are located at a CpG island. In a particular embodiment, the CpG site(s) is/are located at the promoter region. In a particular embodiment, the CpG site(s) is/are located at the gene body. In a particular embodiment, the CpG site(s) is/are located at a CpG shore. In a particular embodiment, the CpG site(s) is/are located at both at a CpG island and a CpG shore. In a particular embodiment, the CpG site(s) is/are located at the N-shore, at the S-shore or at both the N- and the S-shores of said gene(s). The term “promoter region”, as used herein, refers to an upstream region of DNA that initiates transcription of a particular gene. The term “CpG island”, as used herein, relates to a DNA sequence, generally in a window of 200 to 2000 bp, with a GC content greater than 50% and an observed:expected CpG ratio of more than 0.6. The term “gene body” (also referred to as “body” in the present invention) refers to the entire gene from the transcription start site to the end of the transcript. The term “CpG shore”, as used herein, relates to the DNA sequences, up to 2 kb long, flanking a CpG island and showing a comparatively low GC density.

The start and end positions of the promoter, CpG island and shore/CpG island/shore of the genes of the present invention are depicted in Table 1.

TABLE 1
Start and end positions of the CpG island, of the shores flanking the CpG island
and of the promoter regions in the BCAT1, TRIM58, CDO1 and ZNF177 genes.

| | Island | | Shore/Island/Shore | | Start promoter | End promoter |
|---|---|---|---|---|---|---|
| Gene | start | end | start | end | TSS1500 | 1st exon |
| BCAT1 | 25055599 | 25056246 | 25053599 | 25058246 | 25103643 | 25102072 |
| TRIM58 | 248020330 | 248021252 | 248018330 | 248026252 | 248019234 | 248020812 |
| CDO1 | 115151548 | 115152713 | 115149548 | 115152713 | 115153223 | 115152019 |
| ZNF177 | 9473589 | 9474001 | 9471589 | 9476001 | 9472210 | 9476402 |

Island start and island end indicate, respectively, the starting and ending positions of the CpG island by reference to the chromosome numbering according to Infinium HumanMethylation450 BeadChip, Manifest v1.2 or according to UCSC database, as in Genome Reference Consortium Human Build 37 (GRCh37) and UCSC hg19 as released on February 2009 (hereafter referred to as Infinium/UCSC).

Shore/Island/Shore start indicates the starting position of the shore 5′ to the CpG island by reference to the chromosome numbering according to lnfinium/UCSC. Shore/Island/Shore end indicates the end position of the shore located 3′ with respect of the CpG island by reference to the chromosome numbering according to Infinium/UCSC. The end position of the shore located 5′ of the CpG island is the position adjacent in 5′ to the island start position. The start position of the shore located 3′ of the CpG island is the position adjacent in 3′ to the island end position. Start promoter (TSS1500) indicates the start position of the promoter region by reference to the chromosome numbering according to Infinium/UCSC.

End promoter (1st exon) indicates the last position of the first exon of the gene, which is adjacent to the last position of the promoter, by reference to the chromosome numbering as indicated above (lnfinium/UCSC).

In a preferred embodiment of any one of the methods described above of the first aspect of the invention, the methylation level is determined at one or more of the CpG site(s) comprised in SEQ ID NO 1-3 for determining BCAT1's methylation, in SEQ ID NO 4-8 for determining TRIM58's methylation, in SEQ ID NO 9-11 for determining CDO1's methylation, and in SEQ ID NO 12-15 for determining ZNF177's methylation.

In a more preferred embodiment of any one of the methods described above of the first aspect of the invention, the methylation level of any of BCAT1, TRIM58, CDO1, ZNF177 and combinations thereof, is determined at one or more of the CpG site(s) of said genes, and the position(s) of the CpG site(s) is(are) selected from the ones depicted in Table 2 for BCAT1, Table 3 for TRIM58, Table 4 for CDO1 and Table 5 for ZNF177. In Tables 2-5 the indicated positions correspond to the C nucleotide of a CpG site according to MAPINFO/Illumina Infinium HumanMethylation450 BeadChip, Manifest v1.2 or according to UCSC database, as in Genome Reference Consortium Human Build 37 (GRCh37) and UCSC hg19 as released on February 2009.

In a particular embodiment according to any one of the embodiments of the first aspect of the invention mentioned above, in particular the ones described in the two previous paragraphs, the methylation of one or more of the genes BCAT1, TRIM58, CDO1 and ZNF177, is determined at at least two CpG sites, at least 3, at least 4, at least 5, at least 6, at least 7, at least 8, at least 9, at least 10, at least 12, at least 15 CpG sites or at all CpG sites of said gene(s). Preferably, the methylation level of said gene(s) is determined as the average value of said at least 2, 3, 4, 5, 6, 7, 8, 9, 10, 12, 15 or all CpG sites, and more preferably as the average value of all CpG sites.

TABLE 2
Preferred CpG sites of BCAT1 gene

| | Position | | |
|---|---|---|---|
| targetID | MAPINFO | ucscRefGene_NAME | CpG content |
| cg15990629 | 25054873 | BCAT1 | N_Shore |
| cg08980987 | 25054905 | BCAT1 | N_Shore |
| cg21172322 | 25055108 | BCAT1 | N_Shore |
| cg01494454 | 25055214 | BCAT1 | N_Shore |
| cg02585702 | 25055262 | BCAT1 | N_Shore |
| cg08724310 | 25055304 | BCAT1 | N_Shore |
| cg20342079 | 25055381 | BCAT1 | N_Shore |
| cg22229906 | 25055421 | BCAT1 | N_Shore |
| cg22814146 | 25055518 | BCAT1 | N_Shore |
| cg04543413 | 25055676 | BCAT1 | Island |
| | 25055938 | BCAT1 | Island |
| | 25055948 | BCAT1 | Island |
| | 25055957 | BCAT1 | Island |
| | 25055959 | BCAT1 | Island |
| | 25055961 | BCAT1 | Island |
| cg20399616 | 25055967 | BCAT1 | Island |
| | 25055978 | BCAT1 | Island |
| cg23930313 | 25056083 | BCAT1 | Island |
| cg23792314 | 25056243 | BCAT1 | Island |

TABLE 3
Preferred CpG sites of TRIM58 gene

| | Position | | |
|---|---|---|---|
| targetID | MAPINFO | ucscRefGene_NAME | CpG content |
| cg04902327 | 248019234 | TRIM58 | N_Shore |
| cg15094634 | 248019757 | TRIM58 | N_Shore |
| cg04982874 | 248019816 | TRIM58 | N_Shore |
| cg26052730 | 248020331 | TRIM58 | Island |
| cg20855565 | 248020350 | TRIM58 | Island |
| cg10983544 | 248020377 | TRIM58 | Island |
| cg20429172 | 248020436 | TRIM58 | Island |
| cg20810478 | 248020632 | TRIM58 | Island |
| cg26157385 | 248020641 | TRIM58 | Island |
| | 248020671 | TRIM58 | Island |
| | 248020680 | TRIM58 | Island |
| | 248020688 | TRIM58 | Island |
| cg23054189 | 248020692 | TRIM58 | Island |
| | 248020695 | TRIM58 | Island |
| cg20146541 | 248020697 | TRIM58 | Island |
| | 248020704 | TRIM58 | Island |
| | 248020707 | TRIM58 | Island |
| | 248020713 | TRIM58 | Island |
| cg07533148 | 248020812 | TRIM58 | Island |
| cg16021909 | 248021091 | TRIM58 | Island |
| cg09789636 | 248021163 | TRIM58 | Island |

TABLE 4
Preferred CpG sites of CDO1 gene

| | Position | | |
|---|---|---|---|
| targetID | MAPINFO | ucscRefGene_NAME | CpG content |
| cg06682875 | 115150172 | CDO1 | N_Shore |
| cg07712493 | 115151427 | CDO1 | Island |
| cg07405021 | 115152019 | CDO1 | Island |
| cg16265906 | 115152326 | CDO1; CDO1 | Island |
| cg12880658 | 115152386 | CDO1; CDO1 | Island |
| cg16707405 | 115152413 | CDO1 | Island |
| cg02792792 | 115152420 | CDO1 | Island |
| cg14470895 | 115152431 | CDO1 | Island |
| cg23180938 | 115152485 | CDO1 | Island |
| cg08516516 | 115152492 | CDO1 | Island |
| | 115152466 | CDO1 | Island |
| | 115152468 | CDO1 | Island |
| | 115152475 | CDO1 | Island |
| | 115152484 | CDO1 | Island |
| cg11036833 | 115152494 | CDO1 | Island |
| | 115152496 | CDO1 | Island |
| | 115152503 | CDO1 | Island |
| | 115152509 | CDO1 | Island |
| | 115152522 | CDO1 | Island |
| cg07644368 | 115152785 | CDO1 | S_Shore |
| cg16198692 | 115152835 | CDO1 | S_Shore |
| cg04676799 | 115152938 | CDO1 | S_Shore |
| cg23029474 | 115153223 | CDO1 | S_Shore |

TABLE 5
Preferred CpG sites of ZNF177 gene

| | Position | | |
|---|---|---|---|
| targetID | MAPINFO | ucscRefGene_NAME | CpG content |
| cg09492640 | 9472210 | ZNF177 | N_Shore |
| cg14323854 | 9473058 | ZNF177 | N_Shore |
| cg19275200 | 9473240 | ZNF177 | N_Shore |
| cg05250458 | 9473565 | ZNF177 | N_Shore |
| cg05928342 | 9473598 | ZNF177 | Island |
| | 9473668 | ZNF177 | Island |
| cg13703871 | 9473674 | ZNF177 | Island |
| cg08065231 | 9473684 | ZNF177 | Island |
| cg09578475 | 9473688 | ZNF177 | Island |
| cg07788092 | 9473691 | ZNF177 | Island |
| cg09643544 | 9473696 | ZNF177; ZNF 177 | Island |
| | 9473715 | ZNF177; ZNF 177 | Island |
| cg12089570 | 9473715 | ZNF177; ZNF 177 | Island |
| cg24189904 | 9473781 | ZNF177 | Island |
| cg17283453 | 9473880 | ZNF177 | Island |
| cg14737994 | 9474128 | ZNF177 | S_Shore |

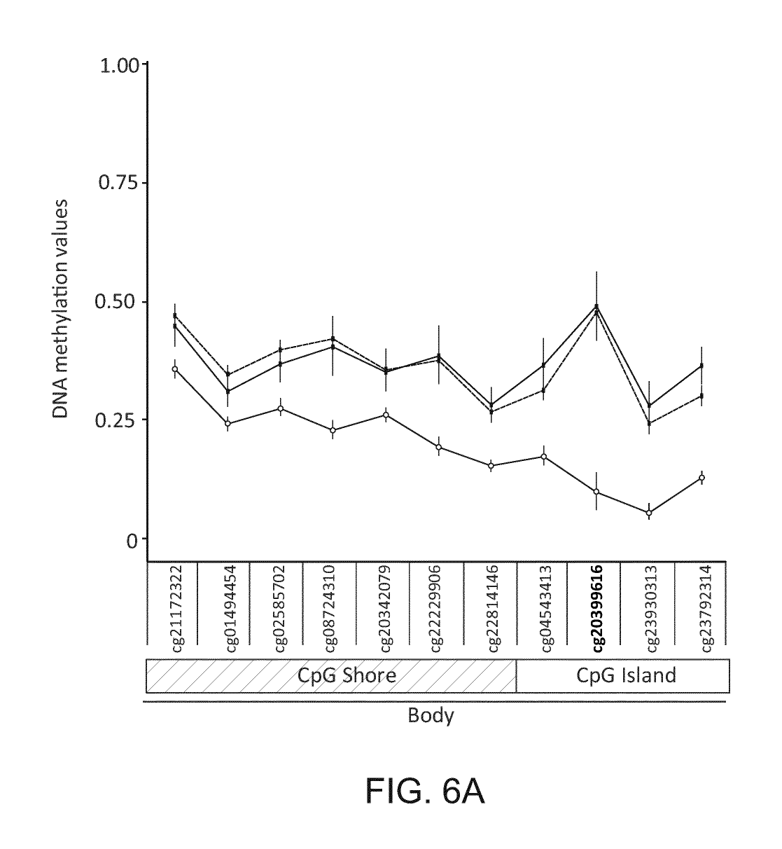

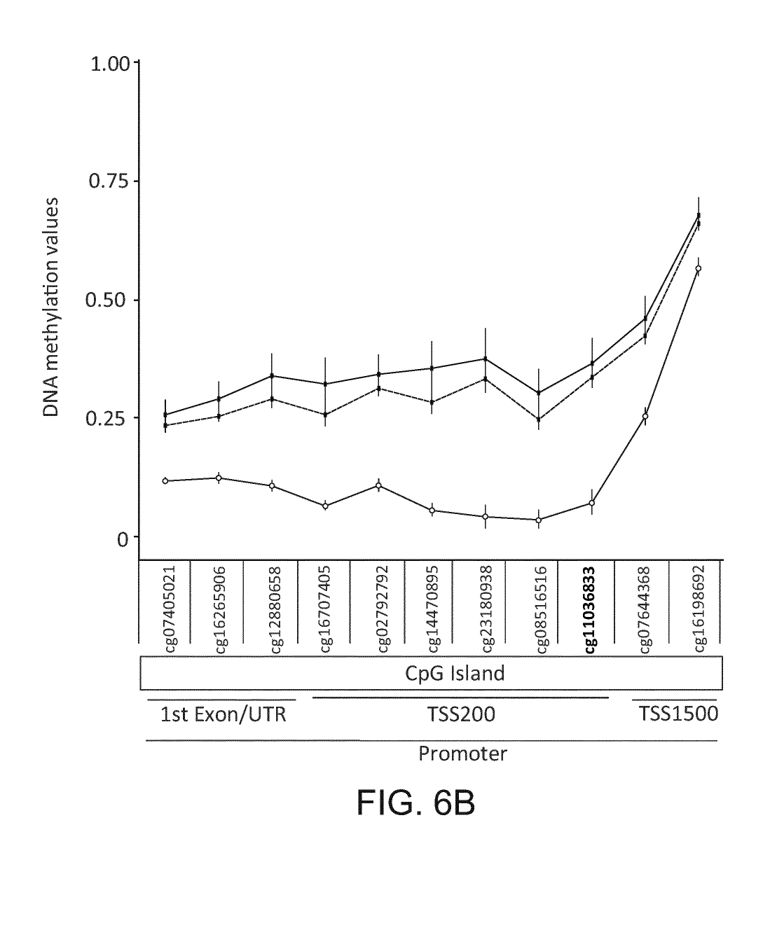

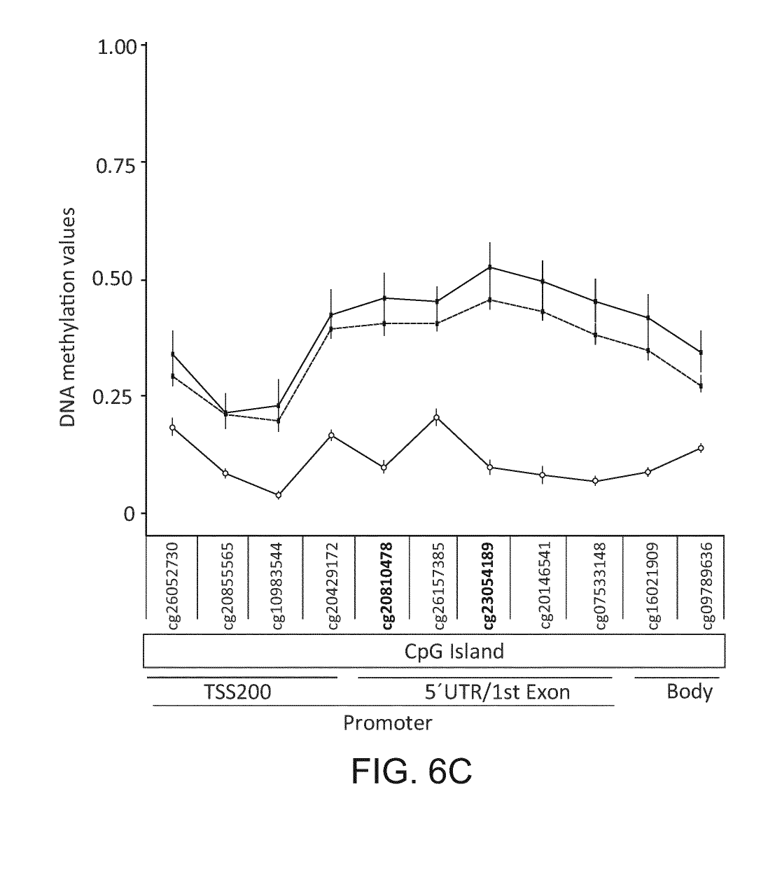

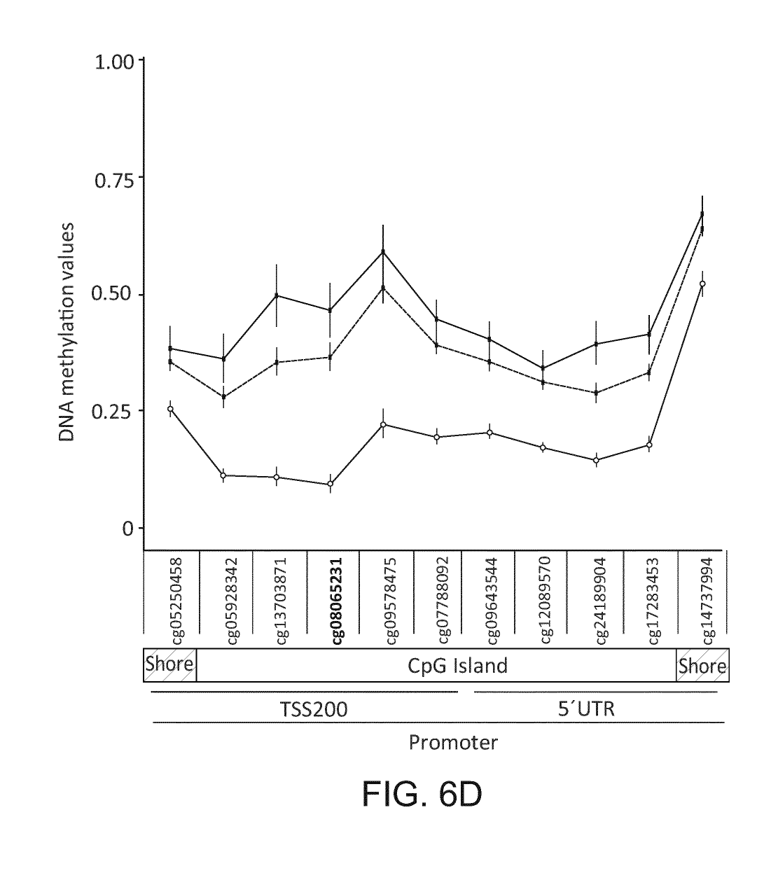

In an even more preferred embodiment of the any one of the methods of the first aspect of the invention described above, the methylation level of any of BCAT1, TRIM58, CDO1, ZNF177 genes or combinations thereof is determined at one or more, preferably all, CpG site(s), selected from the ones depicted in Table 6, which are located in CpG islands. As shown in the examples of the present application, see FIG. 6, the CpG site(s) of Table 6 are the ones given the statistically significant higher degree of methylation in test samples compared to control samples.

TABLE 6
Most preferred CpG sites of BCAT1, TRIM58, CDO1, ZNF177

| NAME | TargetID | Group |
|---|---|---|
| BCAT1 | cg2039961 6 | Body (Island) |
| TRIM58 | cg23054189, cg07533148, | Promoter (Island) |
| | cg20810478, cg16021909 | |
| ZNF177 | cg08065231 | Promoter (Island) |
| CDO1 | cg11036833 | Promoter (Island) |

In a particular embodiment of the first aspect of the present invention, the method of detecting the methylation of any of the genes BCAT1, TRIM58, CDO1, ZNF177 or combinations thereof of two, three or four genes, as described above in the methods of the first aspect of the invention, comprises the steps of: (a) isolating DNA from a biological sample; (b) treating the isolated DNA with bisulfite; (c) amplifying the treated DNA using primers capable of amplifying a fragment comprising the CpG site(s) of the above-mentioned genes; and (d) subjecting the product amplified in step (c) to pyrosequencing to determine the methylation of the gene(s). In a preferred embodiment, the primers for the pyrosequencing are the ones depicted in Table 7 (below) depending on the genes to be analysed, i.e. primers comprising SEQ ID NO 16-18 for determining the methylation level of BCAT1, primers comprising SEQ ID NO 19-21 for determining the methylation level of TRIM58, primers comprising SEQ ID NO 22-24 for determining the methylation level of ZNF177, primers comprising SEQ ID NO 25-27 for determining the methylation level of CDO1. In a more preferred embodiment, the methylation of the four-gene combination is determined, and more preferably in BAS samples. If the POC is to be determined, the algorithm of formula V is used.

The use of the methylated genes described above allows early diagnosis of lung cancer. Thus, a second aspect of the invention refers to a biomarker (referred to as biomarker of the invention) for in vitro lung cancer diagnosis, wherein the biomarker comprises a methylated gene selected from the group consisting of BCAT1, TRIM58, ZNF177, CDO1 and combinations thereof. That is, the biomarker comprises a methylated gene, wherein the gene is selected from one gene, a two-gene combination, a three-gene combination or a four-gene combination, and wherein the gene(s) is(are) selected from the group consisting of BCAT1, TRIM58, ZNF177 and CDO1. As used herein, “methylated gene” refers to a gene containing one or more methylated CpG site(s).

In a particular embodiment of the second aspect of the invention, the biomarker is a methylated gene selected from BCAT1, TRIM58, CDO1 or ZNF177. More preferably the biomarker is methylated BCAT1 gene or methylated TRIM58 gene.

In another embodiment of the second aspect of the invention, the biomarker comprises a methylated two-gene combination wherein the two genes are selected from the group consisting of BCAT1, TRIM58, CDO1 and ZNF177. In a particular embodiment, the two genes are selected from the group consisting of BCAT1, TRIM58, CDO1 and ZNF177, wherein one of said genes is BCAT1. In another embodiment, the two genes are selected from the group consisting of BCAT1, TRIM58, CDO1 and ZNF177, wherein one of said genes is TRIM58. In another embodiment, the two genes are selected from the group consisting of BCAT1, TRIM58, CDO1 and ZNF177, wherein one of said genes is ZNF177. In another embodiment, the methylation level is determined in two genes selected from the group consisting of BCAT1, TRIM58, CDO1 and ZNF177, wherein one of said genes is CDO1. In a preferred embodiment, the two-gene combination is selected from BCAT1 and TRIM58; BCAT1 and ZNF177; BCAT1 and CDO1; TRIM58 and ZNF177; TRIM58 and CDO1; or ZNF177 and CDO1. Preferably, the methylated two-gene combination is selected from BCAT1 and TRIM58, BCAT1 and ZNF177, or BCAT1 and CDO1.

In another embodiment of the second aspect of the invention, the biomarker comprises a methylated three-gene combination wherein the three genes are selected from the group consisting of BCAT1, TRIM58, CDO1 and ZNF177. In a particular embodiment, the three genes are selected from the group consisting of BCAT1, TRIM58, CDO1 and ZNF177, wherein one of said genes is BCAT1. In another embodiment, the three genes are selected from the group consisting of BCAT1, TRIM58, CDO1 and ZNF177, wherein one of said genes is TRIM58. In another embodiment, the three genes are selected from the group consisting of BCAT1, TRIM58, CDO1 and ZNF177, wherein one of said genes is ZNF177. In another embodiment, the methylation level is determined in three genes selected from the group consisting of BCAT1, TRIM58, CDO1 and ZNF177, wherein one of said genes is CDO1. In a preferred embodiment, the three-gene combination is selected from the group consisting of BCAT1, TRIM58 and ZNF177; BCAT1, TRIM58 and CDO1; BCAT1, ZNF177 and CDO1; and TRIM58, ZNF177 and CDO1. Preferably, the methylated three-gene combination is selected from BCAT1, TRIM58 and ZNF177; BCAT1, TRIM58 and CDO1; or BCAT1, ZNF177 and CDO1.

In a further embodiment of the second aspect of the invention, the biomarker comprises a methylated four-gene combination, wherein the genes are BCAT1, TRIM58, CDO1 and ZNF177. The advantages of the biomarkers comprising a combination of genes for use in lung cancer diagnosis are similar to the ones already described in detail in the first aspect of the invention for the embodiment in which the methylation level of the two-, three- or four-gene combination is determined.

In a particular embodiment according to any one of the embodiments described in the previous five paragraphs, the BCAT1 gene comprises any one of sequences SEQ ID NO 1-3, the TRIM58 gene comprises any one of sequences SEQ ID NO 4-8, the CDO1 gene comprises any one of sequences SEQ ID NO 9-11, the ZNF177 gene comprises any one of sequences SEQ ID NO 12-15. In a preferred embodiment of any one of the embodiments of this paragraph each gene is represented by any one of the mentioned sequences.

In a preferred embodiment according to any one of the embodiments described in the previous six paragraphs, the methylated gene contains one or more methylated CpG site(s), and the position(s) of said one or more CpG site(s) is(are) selected from the ones depicted in Tables 2-5. In a more preferred embodiment, said one or more methylated CpG site(s) is(are) selected from the ones depicted in Table 6.

In a particular embodiment according to any one of the embodiments of the second aspect of the invention, in particular the ones of the previous paragraph, the methylated gene contains at least two methylated CpG sites, at least 3, at least 4, at least 5, at least 6, at least 7, at least 8, at least 9, at least 10, at least 12, at least 15 methylated CpG sites or all the CpG sites are methylated, preferably, all the CpG site(s) are methylated.

The second aspect of the invention also refers to a methylated gene selected from the group consisting of BCAT1, TRIM58, ZNF177 and CDO1, or a methylated two-gene combination, a methylated three-gene combination or a methylated four-gene combination of said genes, for use as a biomarker for in vitro lung cancer diagnosis, preferably early lung cancer diagnosis. “Methylated gene combination” refers to a gene combination containing one or more methylated CpG sites. In particular embodiments, the methylated genes and combinations, and the methylated CpG sites are the ones described in the previous eight paragraphs for the biomarker of the invention.

The method of any of the embodiments described in the first aspect of the present invention can be carried out using a kit. Thus, a third aspect of the present invention refers to a kit for the in vitro diagnosis of lung cancer in a subject, comprising:
primers for amplifying a CpG-containing nucleic acid of a gene, and/or
means for detecting the presence of methylated CpG site(s) in said amplified nucleic acid,
   wherein the gene is selected from one gene, a two-gene combination, a three-gene combination or a four-gene combination, and
   wherein the gene(s) is(are) selected from the group consisting of BCAT1, TRIM58, ZNF177 and CDO1.

In a particular embodiment, the third aspect of the invention refers to a kit to carry out the method according to any one of the embodiments described in the first aspect of the invention, comprising:
primers for amplifying a CpG-containing nucleic acid of a gene, and/or
means for detecting the presence of methylated CpG site(s) in said amplified nucleic acid,
   wherein the gene is selected from one gene, a two-gene combination, a three-gene combination or a four-gene combination, and
   wherein the gene(s) is(are) selected from the group consisting of BCAT1, TRIM58, ZNF177 and CDO1.

In a particular embodiment of the third aspect of the invention according to any of the two previous paragraphs, the kit comprises primers for amplifying a CpG-containing nucleic acid of a gene selected from BCAT1 or TRIM58 or CDO1 or ZNF177. Preferably the gene is BCAT1 or TRIM58, more preferably the gene is BCAT1. In another embodiment, the kit comprises primers for amplifying two CpG-containing nucleic acids, one of each gene of the two-gene combinations described in the first aspect of the invention, i.e. the two-gene combination is selected from BCAT1 and TRIM58; BCAT1 and ZNF177; BCAT1 and CDO1; TRIM58 and ZNF177; TRIM58 and CDO1; or ZNF177 and CDO1, preferably the two-gene combinations contains BCAT1. In another embodiment, the kit comprises primers for amplifying three CpG-containing nucleic acids, one of each gene of the three-gene combinations described in the first aspect of the invention, i.e. the three-gene combination is selected from BCAT1, TRIM58 and ZNF177; BCAT1, TRIM58 and CDO1; BCAT1, ZNF177 and CDO1; or TRIM58, ZNF177 and CDO1, preferably the three-gene combinations contains BCAT1. In another embodiment, the kit comprises primers for amplifying four CpG-containing nucleic acids, in particular a CpG-containing nucleic acid of BCAT1 gene, a CpG-containing nucleic acid of TRIM58 gene, a CpG-containing nucleic acid of CDO1 gene, and a CpG-containing nucleic acid of ZNF177 gene.

In a particular embodiment, the kit of the third aspect of the invention comprises means for detecting the presence of methylated CpG site(s). More preferably, the kit comprises primers for amplifying a CpG-containing nucleic acid of the gene(s) according to any one of the embodiments of the previous paragraph, and means for detecting the presence of methylated CpG site(s) in said amplified nucleic acid(s). In a particular embodiment of the third aspect of the invention according to any of the embodiments described in the previous four paragraphs, the BCAT1 gene comprises any one of sequences SEQ ID NO 1-3, the TRIM58 gene comprises any one of sequences SEQ ID NO 4-8, the CDO1 gene comprises any one of sequences SEQ ID NO 9-11, the ZNF177 gene comprises any one of sequences SEQ ID NO 12-15. In a preferred embodiment of any of the embodiments of this paragraph each gene is represented by any one of the mentioned sequences.

In a preferred embodiment according to any of the embodiments described in the previous five paragraphs, the amplified CpG-containing nucleic acids contains one or more, preferably all, CpG site(s) selected from the CpG sites of the positions depicted in Tables 2-5. In a more preferred embodiment, said one or more, preferably all, CpG site(s) is(are) selected from the ones depicted in Table 6.

The term “primer”, as used herein, refers to a single-stranded DNA or RNA molecule, with up to 30, 25, 20, 19, 18, 17, 16, 15, 14 or 13 bases in length (upper limit). The oligonucleotides of the invention are preferably DNA or RNA molecules of at least 2, 3, 4, 5, 6, 7, 8, 9, 10, 11, 12, or 13 bases in length (lower limit). Ranges of base lengths can be combined in all different manners using the afore-mentioned lower and upper limits, for example at least 2 and up to 30 bases, at least 8 and up to 15 bases, at least 5 and up 15 bases or at least 8 and up to 18 bases. In a preferred embodiment, the sequence of the primers for amplifying the CpG-containing nucleic acid hybridize with CpG-free sites to ensure methylation-independent amplification, i.e. the primers are flanking the CpG sites, one upstream and the other downstream of the CpG site(s) of interest. In a preferred embodiment according to any one of the embodiments of the previous six paragraphs, the primers comprise a sequence selected from SEQ ID NO 16 and 17 for amplifying BCAT1, SEQ ID NO 19 and 20 for amplifying TRIM58, SEQ ID NO 22 and 23 for amplifying ZNF177, and/or SEQ ID NO 25 and 26 for amplifying CDO1 (see Table 7). Preferably the means of detection are also primers, more preferably said primers comprise a sequence selected from SEQ ID NO 18 (for detecting methylated BCAT1), 21 (for detecting methylated TRIM58), 24 (for detecting methylated ZNF177) and/or 27 (for detecting methylated CDO1), depending on which methylated gene(s) is/are to be detected (see Table 7).

TABLE 7
Primer sequences

| Target | | PRIMER SEQUENCE | LENGTH |
|---|---|---|---|
| ID | CpG | F: FORWARD; R: REVERSE; S: SEQUENCING | (bp) |
| BCAT1 | cg20399616 | F [btn]GAGGTTTTTTTTTAAGGGATGTTGGA | 279 |
| | | (SEQ ID NO 16) | |
| | | R TCCAATCCTCCCCCCTTC | |
| | | (SEQ ID NO 17) | |
| | | S AACTAACCATAAAAAAACTAC | |
| | | (SEQ ID NO 18) | |
| TRIM58 | cg23054189 | F TGTTYGGTGTGTTTGGATTTTTTGTAG | 201 |
| | | (SEQ ID NO 19) | |
| | | R [btn]CACRCTCTCCACCAAACCC | |
| | | (SEQ ID NO 20) | |
| | | S ATAGTTTTTGTTTTAGGT | |
| | | (SEQ ID NO 21) | |
| ZNF177 | cg08065231 | F AATGTGYGAGTTGGGTAGTTTATTTTT | 122 |
| | | (SEQ ID NO 22) | |
| | | R [btn]CTACTAAAACAACAACCCTTTCTCAA | |
| | | (SEQ ID NO 23) | |
| | | S AGTTTATTTTTTTTAGTTGTTGG | |
| | | (SEQ ID NO 24) | |
| CDO1 | cg11036833 | F GTTAAAGTGGGGGAGAGATT | 237 |
| | | (SEQ ID NO 25) | |
| | | R [btn]TCATCCTCCCCAARCCCTTTTAAAC | |
| | | (SEQ ID NO 26) | |
| | | S GGGTTTTTGGGAAGG | |
| | | (SEQ ID NO 27) | |

Suitable kits may include primers for amplification and/or means for detection, and various reagents for use in accordance with the present invention in suitable containers and packaging materials, including tubes, vials, and shrink-wrapped and blow-moulded packages. In one embodiment, the kit includes reagents for amplifying and detecting methylation. Optionally, the kit includes sample preparation reagents and/or articles (e.g. tubes) to extract nucleic acids from samples. Additionally, the kits of the invention can contain instructions for the simultaneous, sequential or separate use of the different components which are in the kit.

The method, the biomarker and the kit according to the present invention make it possible to diagnose lung cancer at an early stage in an accurate and rapid manner compared to conventional methods. Thus, a fourth aspect of the invention refers to the use of a method according to any one of the embodiments of the first aspect of the invention described above, for in vitro lung cancer diagnosis. The fourth aspect of the invention also refers to the use of a biomarker according to any one of the embodiments of the second aspect of the invention, for in vitro lung cancer diagnosis. The fourth aspect of the invention also refers to the use of a biomarker for in vitro lung cancer diagnosis, wherein the biomarker is a biomarker according to any one of the embodiments of the second aspect of the invention. The fourth aspect of the invention also refers to the use of a kit according to any one of the embodiments of the third aspect of the invention, for in vitro lung cancer diagnosis. Preferably the use of the fourth aspect of the invention is an in vitro use. More preferably, the use is for in vitro diagnosis of lung cancer in early stage.

Terms used in context of the aspects second to fourth, have the meaning as defined in the first aspect of the present invention.

All publications mentioned herein are hereby incorporated in their entirety by reference. While the foregoing invention has been described in some detail for purposes of clarity and understanding, it will be appreciated by one skilled in the art from a reading of this disclosure that various changes in form and detail can be made without departing from the true scope of the invention and appended claims.

The examples below serve to further illustrate the invention, and to provide those of ordinary skill in the art with a complete disclosure and description of how the methods and uses herein are carried out, and are not intended to limit the scope of the present invention.

### EXAMPLES

### Example 1. Samples, Procedure, Statistics

### Samples, Cohorts

Methylation of the biomarkers was conducted by pyrosequencing in four independent cohorts. Lung cohorts were obtained from different institutions in Spain. i) A total of 201 FFPE samples were obtained from Health Institute Carlos III ° SCUD, Madrid and Centre for Applied Medical Research/Hospital of the University of Navarre, (CIMA/CUN) Pamplona. Regarding minimally invasive samples, ii) 80 BAS and iv) 98 sputums were obtained from Catalan Institute of Oncology and Bellvitge University Hospital, Barcelona. iii) 111 BAL came from CIMA, Pamplona and Hospital of Talavera de la Reina, Talavera de la Reina.

All DNA extractions from different specimens were developed and run by the same technicians to avoid interlaboratory variation. The study was approved by the corresponding institutional review board and patients signed up the informed consent to participate.

The TCGA (lung adenocarcinoma LUAD or Lung squamous cell carcinoma LUSC) cohort was previously described in Sandoval et al. J Clin Oncol 2013; 31:4140-7. The main clinic-characteristics of the different cohorts are described in Table 8.

### Preparation of Lung Specimens

DNA was extracted from minimally and non-invasive specimens using a standard phenol chloroform extraction method. DNA from FFPE tissue blocks was extracted from two sequential unstained sections, each 10 μm thick. For each sample of tumor tissue, subsequent sections were stained with hematoxylin and eosin for histological confirmation of the presence (>50%) of tumor cells. Unstained tissue sections were deparaffinized, and DNA was extracted using the same protocol as for minimally invasive specimens. Extracted DNA was checked for integrity and quantity with 1.3% agarose gel electrophoresis and picogreen quantification, respectively. Bisulfite conversion of 500 ng of DNA for each sample was performed using the EZ DNA Methylation Gold (ZYMO RESEARCH) bisulfite conversion kit according to the manufacturer's recommendation.

### Pyrosequencing

Pyrosequencing analyses to determine CpG methylation level were developed as previously described (Sandoval J. et al., A prognostic DNA methylation signature for stage I non-small-cell lung cancer. J Clin Oncol 2013; 31:4140-7). Briefly, a set of primers for PCR amplification and sequencing were designed using a specific software pack (PyroMark assay design version 2.0.01.15). Primer sequences were designed to hybridize with CpG-free sites to ensure methylation-independent amplification (see Table 7). DNA was converted using the EZ DNA Methylation Gold (ZYMO RESEARCH) bisulfite conversion kit following the manufacturer's recommendations and used as a template for subsequent PCR step. PCR was performed under standard conditions with primers biotinylated ([btn]) to convert the PCR product to single-stranded DNA templates. We used the Vacuum Prep Tool (Biotage, Sweden) to prepare single-stranded PCR products according to manufacturer's instructions. PCR products were observed at 2% agarose gels before pyrosequencing. Pyrosequencing reactions and methylation quantification were performed in a PyroMark Q24 System version 2.0.6 (Qiagen) using appropriate reagents and protocols, and the methylation value was obtained from the average of the CpG dinucleotides included in the sequence analyzed, with a minimum of 3 valid CpGs per primer. Only those average methylation values within the region analyzed with coefficient of variation lower than 1 were accepted as valid. Controls to assess correct bisulfite conversion of the DNA were included in each run, as well as sequencing controls to ensure the fidelity of the measurements.

### Statistical Analysis

Data were summarized by mean, standard deviation, median and first and third quartiles in the case of continuous variables and by relative and absolute frequencies in the case of categorical variables. Differences in expression values and methylation levels among groups were assessed using the non-parametric Wilcoxon rank sum test. Receiver Operating Characteristic (ROC) curves were used to assess the predictive capacity of each marker. Area under the curve (AUC) was computed for each ROC curve, and 95% confidence intervals (CI) were also estimated by bootstrapping with 1000 iterations. A predictive model for each sample type was built including all selected markers in a multivariable logistic regression model. ROC curves and AUC were also computed for the predictive models. Internal validation of the models was performed using 10-fold crossvalidation. The final predictive models were represented in monograms to facilitate their use by clinicians. Sensitivity and specificity were estimated at the optimal cut-off point according to Youden's criterion. Additionally, the sensitivity and specificity curves were estimated for the whole range of predictions of the model to allow for personalized decisions in different clinical scenarios. Globally, a two-tailed p-value of less than 0.05 was considered to indicate statistical significance. P-values were adjusted for multiple comparisons using the FDR procedure by Benjamini and Hochberg. All statistical analyses were performed using R software (version 3.2.0) and the pROC R-package (version 1.7.3).

The inventors generated a mathematical algorithm to calculate the probability of cancer for any sample type and gene combination. The general form of said algorithm was (formula I):

$$Pr ( Cancer ) = \frac{{e}^{a + b * TRIM 58 + c * ZNF 177 + d * CDO 1 + e * BCAT 1}}{1 + {e}^{a + b * TRIM 58 + c * ZNF 177 + d * CDO 1 + e * BCAT 1}} * 100$$

Where the coefficients a, b, c, d and e take the log-odd values of cancer estimated by a multivariable logistic regression model adjusted using maximum likelihood to methylation values data from a specific sample type and a specific combination of the four genes.

### Example 2.—Early Lung Cancer Detection in Minimally-Invasive Respiratory Samples: BAS

One of the most important aspects for early diagnostics is to identify markers associated with cancer using non-invasive or minimally-invasive methods for sample collection. In line, the inventors collected an independent cohort of BAS from patients diagnosed with lung cancer (n=51) and cancer-free patients (n=29) (Table 8). This cohort included different lung cancer subtypes, especially ADC and SCC. The inventors compared by pyrosequencing the median methylation levels and generated ROC curves to assess the performance of each marker independently. Airways fluids from lung cancer patients presented significant differences in DNA methylation levels (FIG. 1A-1D) and high AUCs for all four genes (FIG. 1E-1H).

The inventors analysed the AUC of different combinations of BCAT1, CDO1, TRIM58 and ZNF177 in a logistic regression model yielding significant AUCs higher than or equal than 0.73 in BAS samples (see Table 9). Thus, any of these combinations, and in particular the ones with higher AUC, may be of high value to detect lung cancer in BAS samples.

TABLE 9
AUC of single genes and different combinations
thereof in BAS, BAL and sputum samples.

| Gene | AUC - BAS | AUC - BAL | AUC - ESPUT |
|---|---|---|---|
| BCAT1 | 0.76 | 0.8 | 0.92 |
| TRIM58 | 0.8 | 0.72 | 0.67 |
| ZNF177 | 0.76 | 0.66 | 0.69 |
| CDO1 | 0.73 | 0.65 | 0.67 |
| BCAT1 + TRIM58 | 0.87 | 0.85 | 0.92 |
| BCAT1 + ZNF177 | 0.86 | 0.84 | 0.92 |
| BCAT1 + CDO1 | 0.82 | 0.79 | 0.92 |
| TRIM58 + ZNF177 | 0.86 | 0.73 | 0.68 |
| TRIM58 + CDO1 | 0.83 | 0.72 | 0.74 |
| ZNF177 + CDO1 | 0.83 | 0.73 | 0.76 |
| BCAT1 + TRIM58 + | 0.9 | 0.86 | 0.92 |
| ZNF177 | | | |
| BCAT1 + TRIM58 + | 0.88 | 0.84 | 0.92 |
| CDO1 | | | |
| BCAT1 + ZNF177 + | 0.88 | 0.83 | 0.92 |
| CDO1 | | | |
| TRIM58 + ZNF177 + | 0.88 | 0.74 | 0.76 |
| CDO1 | | | |
| BCAT1 + TRIM58 + | 0.91 | 0.85 | 0.93 |
| ZNF177 + CDO1 | | | |

Combination of BCAT1, CDO1, TRIM58 and ZNF177 in a logistic regression model yielded a significant AUC of 0.91 (95% CI [0.83, 0.98] p<0.001, FIG. 11). Calibration of the model showed no evident deviations from the ideal identity slope (data not shown). Internal validation of the AUC estimate for this model yielded optimism corrected AUC of 0.90, showing high generalization of the predictive capacity of the model for future samples. A nomogram based on the results of this model is proposed as a predictive tool for clinical diagnostic use. Results of the nomogram provide an individual probability (0%-100%) for suffering lung cancer for each patient (FIG. 2). Evaluation of the full range of predictions of the model shows that shifting the cut-off to POC=30% would yield a sensitivity of 100% and a specificity of 65.4% and shifting the cut-off to POC=80% would yield a sensitivity of 71.4% and a specificity of 92.3%. Sensitivity and specificity at the optimal cut-off (POC=63%) were 84.6% and 81.0% respectively (FIG. 1J).

It is important to point out that current protocols for lung cancer diagnosis are based mainly in bronchioalveolar cytology and further lung biopsy. There are cases where the cytology is doubtful or inconclusive. Moreover, there are a notable number of cases where cytology and biopsy are negative for cancer cells, but there is high suspicion of cancer. The present results not only improve the overall prediction accuracy of BAS cytology in this cohort (sensitivity=43.8%, specificity=100%), but also permit a flexible and personalized approach for the clinicians in every possible scenario by simply adapting the cut-off value of the probabilistic model. In this sense, in the studied cohort 24 of 51 tumor samples were misinterpreted as non-tumoral by the cytology test. However, using the predictive four-gene epigenetic signature of the present invention, 19 out of the 24 false negative cytologies (79%) would have been considered as positive setting the threshold at 50% probability of cancer (Table 10).

TABLE 10
Probability of cancer

| Model prediction | |
|---|---|
| (Probability of Cancer) | N^{o} patients |
| 0%-20% | 0 |
| 20%-30% | 1 |
| 30%-40% | 1 |
| 40%-50% | 3 |
| 50%-60% | 0 |
| 60%-70% | 1 |
| 70%-80% | 2 |
| 80%-100% | 16 |

Of note, the majority of them (16 of 24) with a predicted probability of cancer higher than 80%. Also three of them were classified as borderline non-tumor, with a predicted probability of cancer between 40% and 50%. In these three doubtful cases, clinical patient manage would require further additional studies. Thus, the epigenetic signatures described in the present invention, in particular the four-gene epigenetic signature, is a useful clinical diagnostic tool in BAS specimens, especially in doubtful cases.

The concrete values for the coefficients of the general algorithm indicated in Example 1 (formula I) are specified below for particular gene-combinations in the BAS samples. Four-gene signature: Combination of BCAT1, TRIM58, ZNF177 and CDO1 (formula V)

$${Pr ( Cancer )}_{BAS} = \frac{{e}^{- 6.13 + 0.18 * TRIM 58 + 0.30 * ZNF 177 + 0.47 * CDO 1 + 0.59 * BCAT 1}}{1 + {e}^{- 6.13 + 0.18 * TRIM 58 + 0.30 * ZNF 177 + 0.47 * CDO 1 + 0.59 * BCAT 1}} * 100$$

That is, the coefficients are: a=−6.13, b=0.18, c=0.30, d=0.47, e=0.59.

Three-gene signature: Combination of BCAT1, TRIM58 and ZNF177 (formula IV) a=−5.15 b=0.20 c=0.32 d=0 e=0.66

$${Pr ( Cancer )}_{BAS} = \frac{{e}^{- 5.15 + 0.20 * TRIM 58 + 0.32 * ZNF 177 + 0.66 * BCAT 1}}{1 + {e}^{- 5.15 + 0.20 * TRIM 58 + 0.32 * ZNF 177 + 0.66 * BCAT 1}} * 100$$

That is, the coefficients are: a=−5.15, b=0.20, c=0.32, d=0, e=0.66.

Two-gene signature: Combination of BCAT1 and TRIM58 (formula III)

$${Pr ( Cancer )}_{BAS} = \frac{{e}^{- 3.03 + 0.24 * TRIM 58 + 0.55 * BCAT 1}}{1 + {e}^{- 3.03 + 0.24 * TRIM 58 + 0.55 * BCAT 1}} * 100$$

That is, the coefficients are: a=−3.03, b=0.24, c=0, d=0, e=0.55.

One-gene signature: BCAT1 gene (formula II)

$${Pr ( Cancer )}_{BAS} = \frac{{e}^{- 1.86 + 0.63 * BCAT 1}}{1 + {e}^{- 1.86 + 0.63 * BCAT 1}} * 100$$

That is, the coefficients are: a=−1.86, b=0, c=0, d=0, e=0.63.

### Example 3.—Early Lung Cancer Detection in Minimally-Invasive Respiratory Samples: BAL

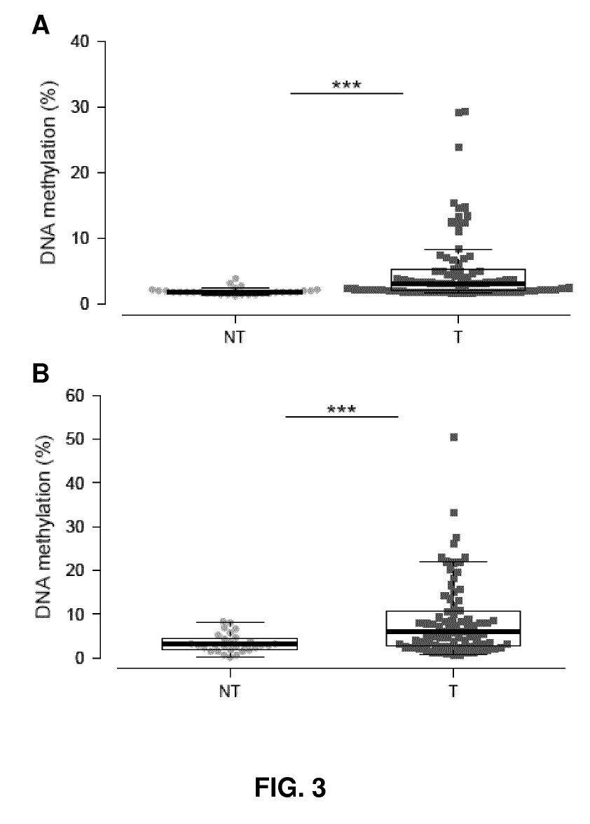

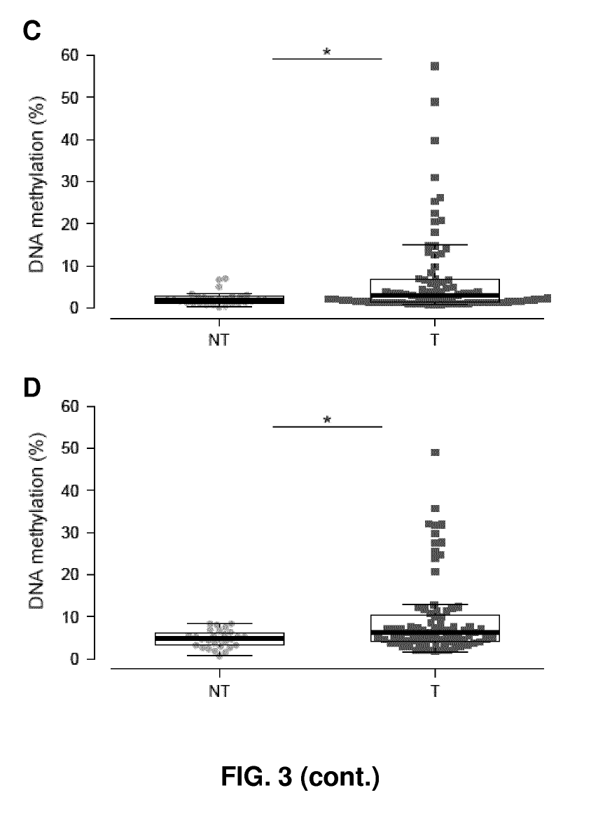

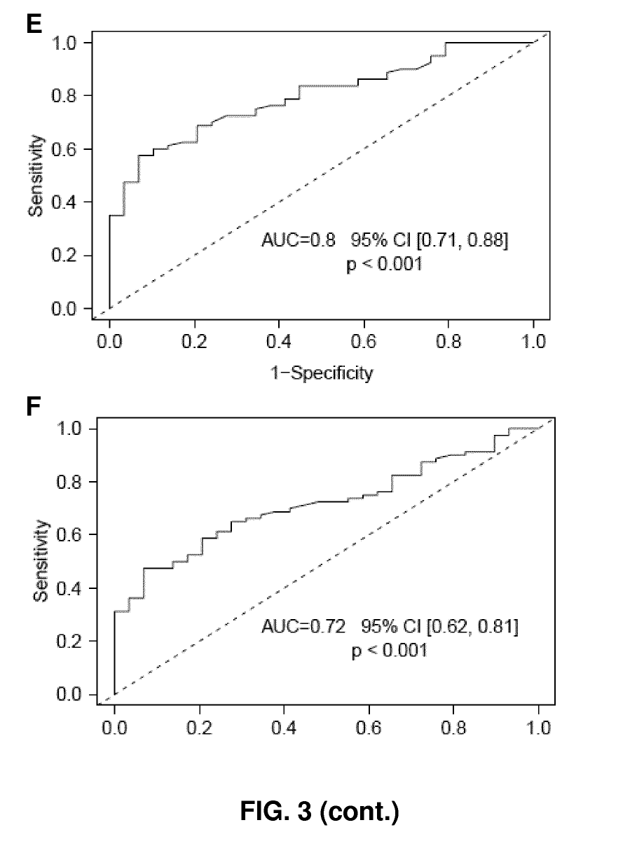

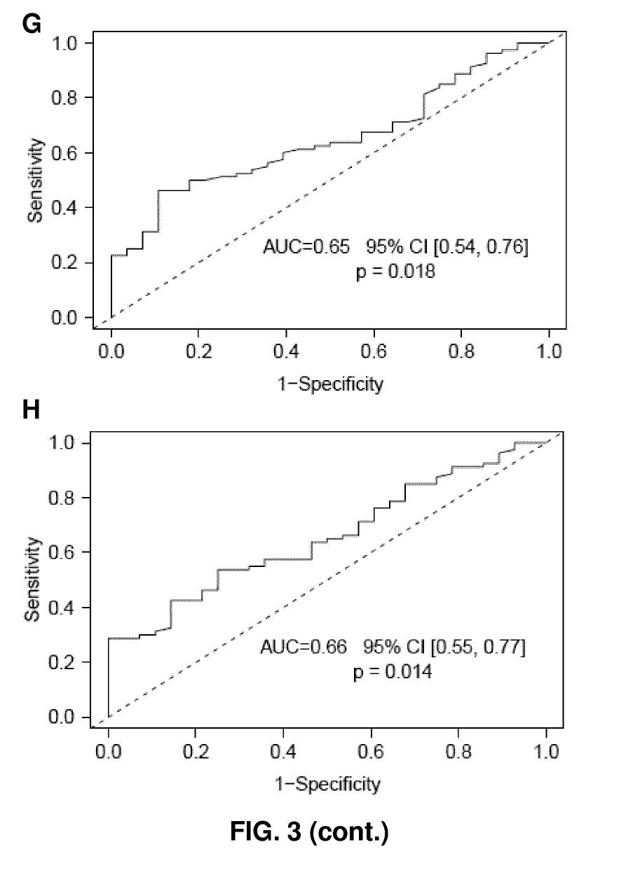

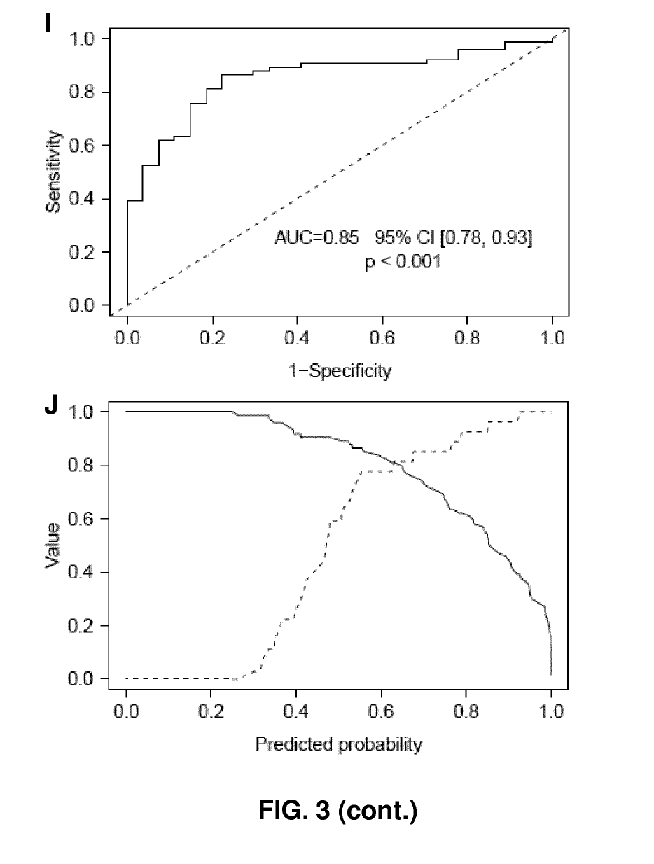

Additionally, the inventors evaluated DNA methylation levels in BAL from patients with lung cancer (n=82) as compared to non-malignant lung diseases (n=29) (Table 8). The methylation levels of the four markers individually were significantly higher in BAL fluid from cancer patients than non-cancer patients (FIG. 3A-3D). AUCs were significant for all four genes with the following values AUC_{BCAT1}=0.80, AUC_{CDO1}=0.65, AUC_{TRIM58}=0.72 and AUC_{ZNF177}=0.66 (FIG. 3E-3H). Combination of the four genes in a logistic regression model achieved a significant AUC of 0.85 (95% CI [0.78, 0.93] p<0.001) (FIG. 31), with an optimism-corrected value of 0.83, confirming that the model is valid. Evaluation of the full range of predictions of the model is also shown (FIG. 3J). The AUC of different combinations of BCAT1, CDO1, TRIM58 and ZNF177 in a logistic regression model yielding significant AUCs higher than or equal to 0.65 in BAL samples (see Table 9). Thus, as in the case with BAS specimens, the different epigenetic signatures of the present invention, in particular the four-gene epigenetic signature, may be of high value to detect lung cancer in BAL samples and may be highly valuable for doubtful patients with negative cytology.

### Example 4.—Early Lung Cancer Detection in Non-Invasive Sputum Samples

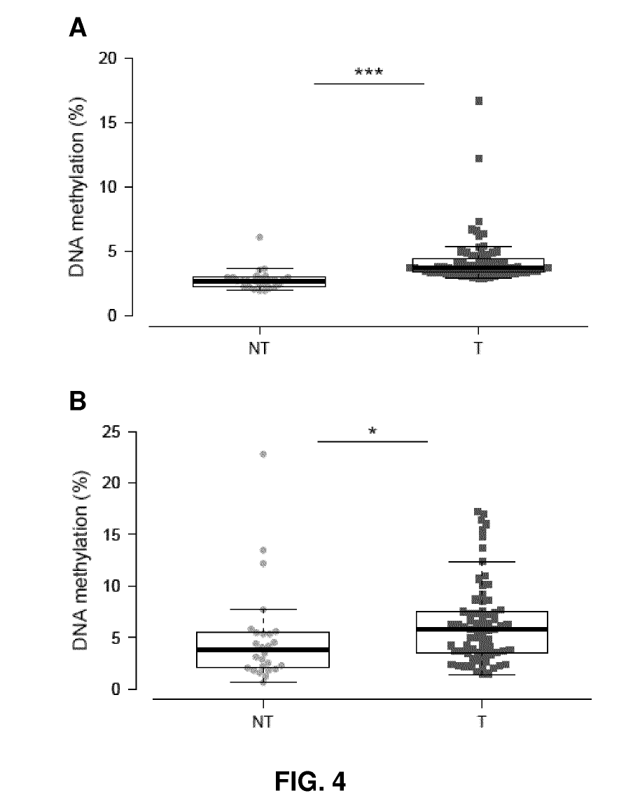

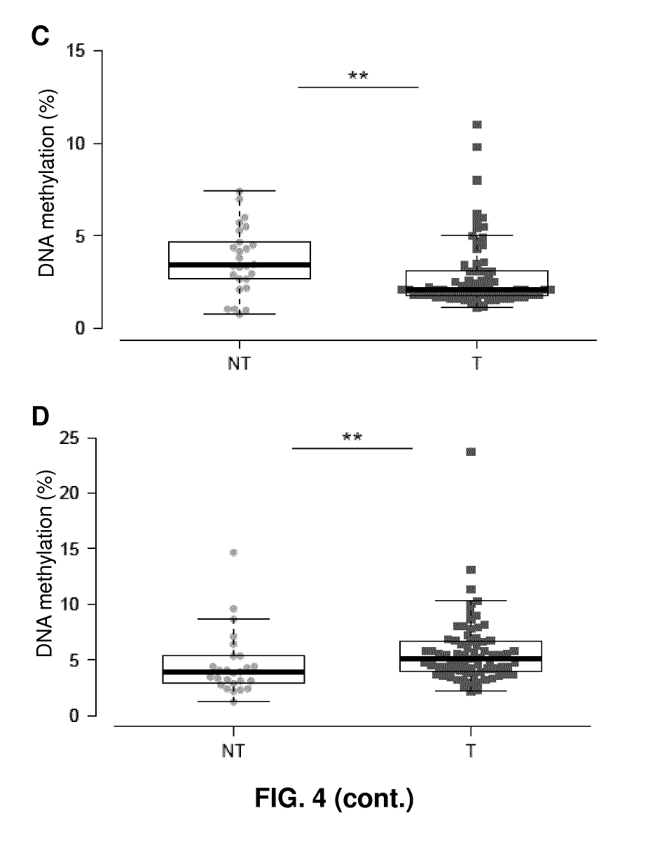

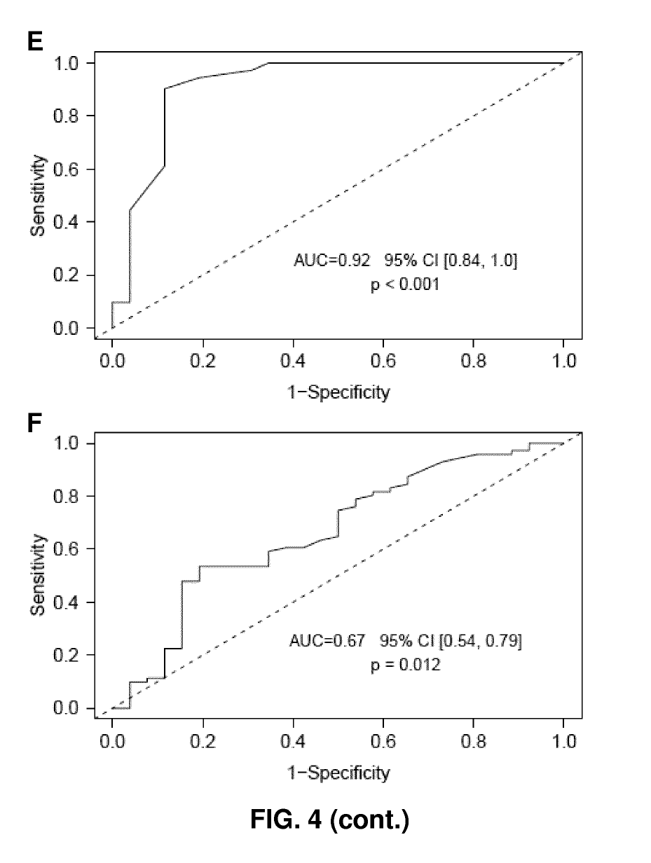

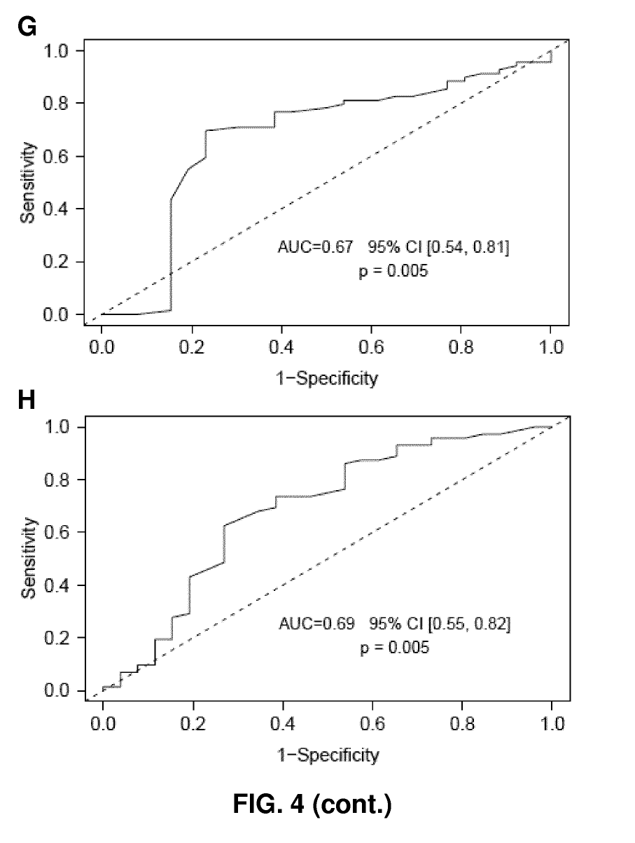

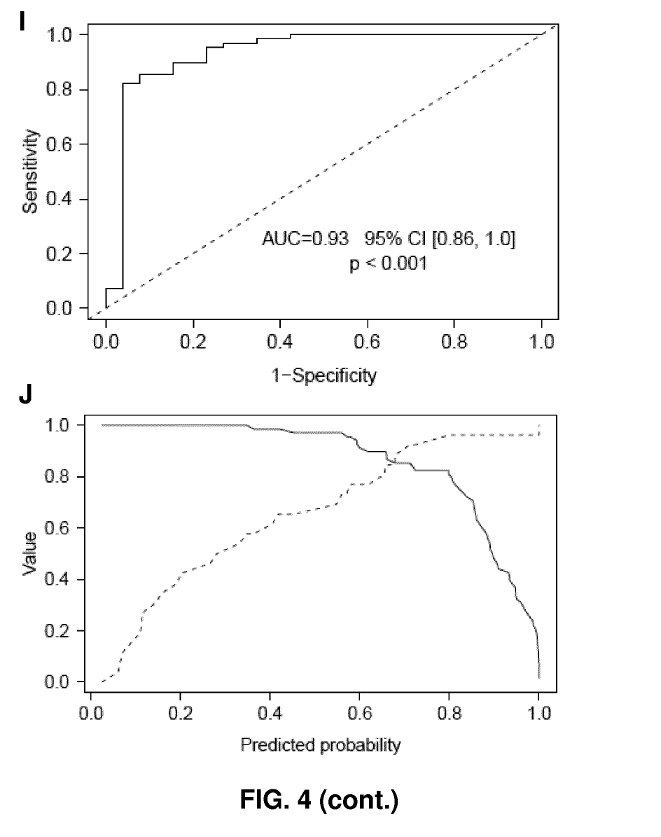

There are many reports that the DNA hypermethylation of various genes can also be detected in the sputum of lung cancer patients. Thus, the methylation level of these 4 markers was examined in sputums samples from 72 lung cancer patients and 26 cancer-free individuals (Table 8). Methylation levels were significantly higher in individuals with lung cancer for all the genes tested, except for CDO1 (FIG. 4A-4D). Individual AUC values were AUC_{BCAT1}=0.92, AUC_{CDO1}=0.67, AUC_{TRIM58}=0.67 and AUC_{ZNF177}=0.69 (FIG. 4E-4H). The combination of logistic regression model yielded an AUC value of 0.93 (95% CI [0.86, 1.0], p<0.001) (FIG. 41). Sensitivity and specificity for the different threshold values of the model are depicted (FIG. 4J). The AUC of different combinations of BCAT1, CDO1, TRIM58 and ZNF177 in a logistic regression model yielding significant AUCs higher than or equal to 0.67 in sputum samples (see Table 9). Thus, as in the case with BAS and BAL specimens, the different epigenetic signatures of the present invention, in particular the 4-gene epigenetic signature, may be of high value to detect lung cancer in sputum samples and may be highly valuable for doubtful patients with negative cytology.

### Example 5.—Early Lung Cancer Detection in Primary Tumors: FFPE

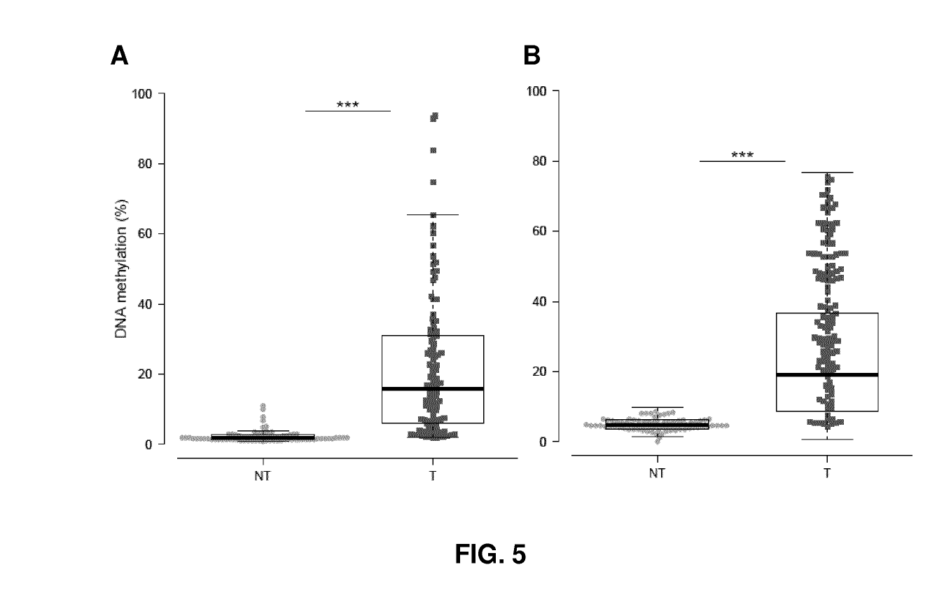

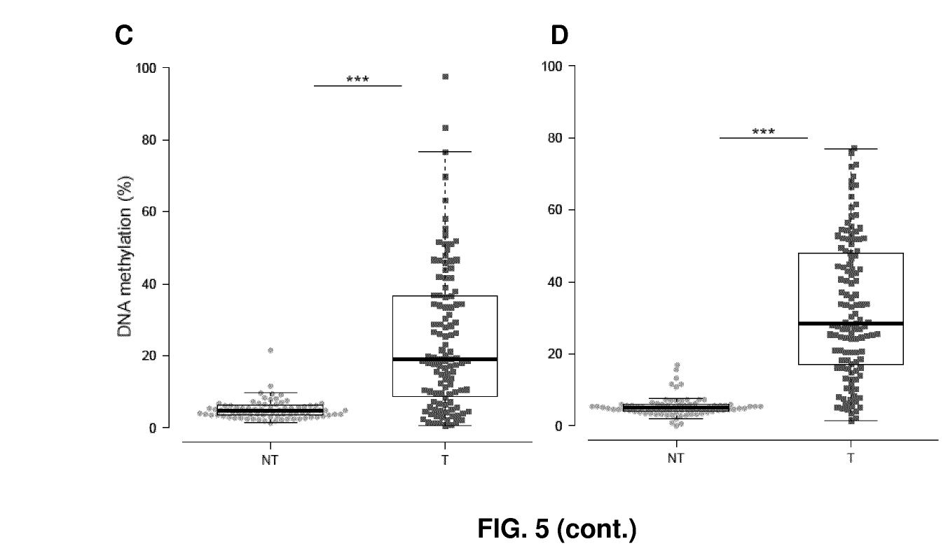

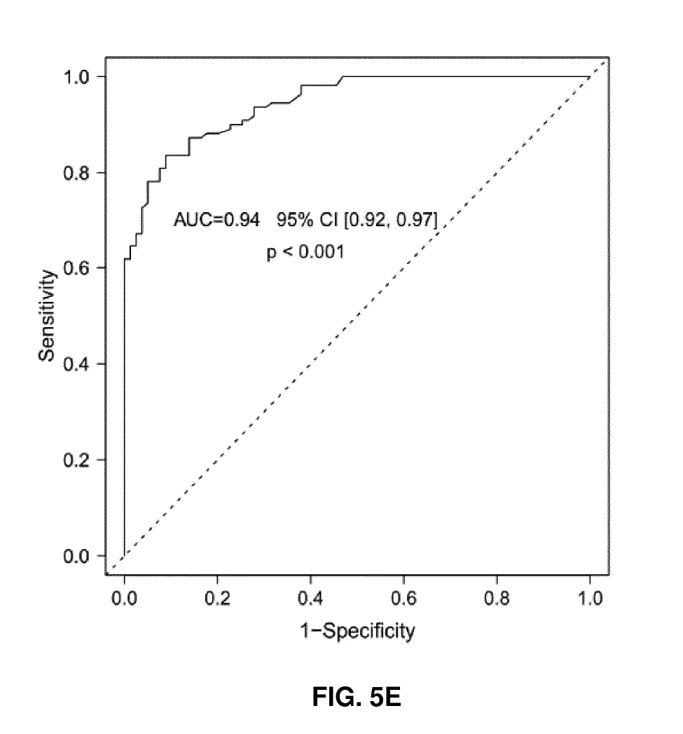

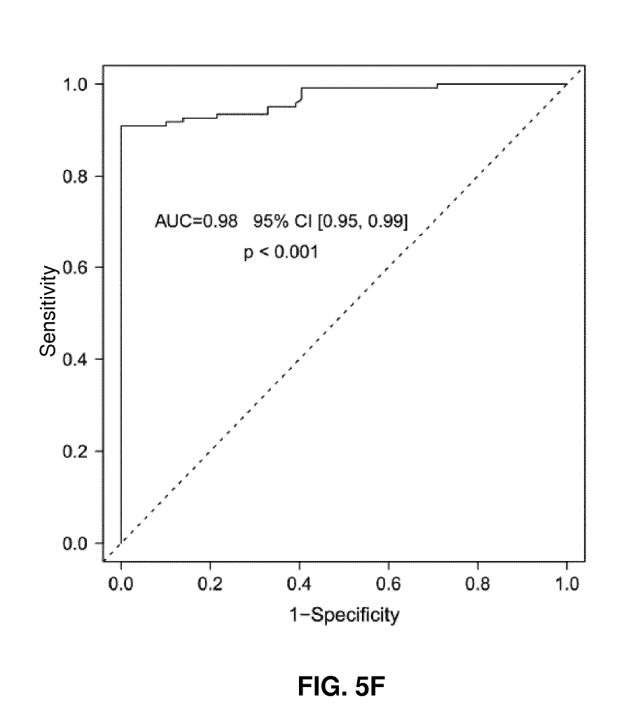

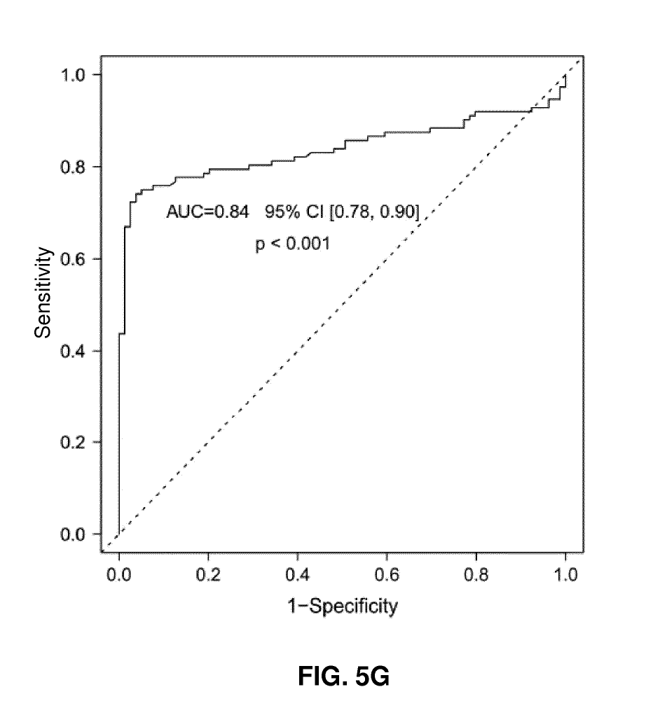

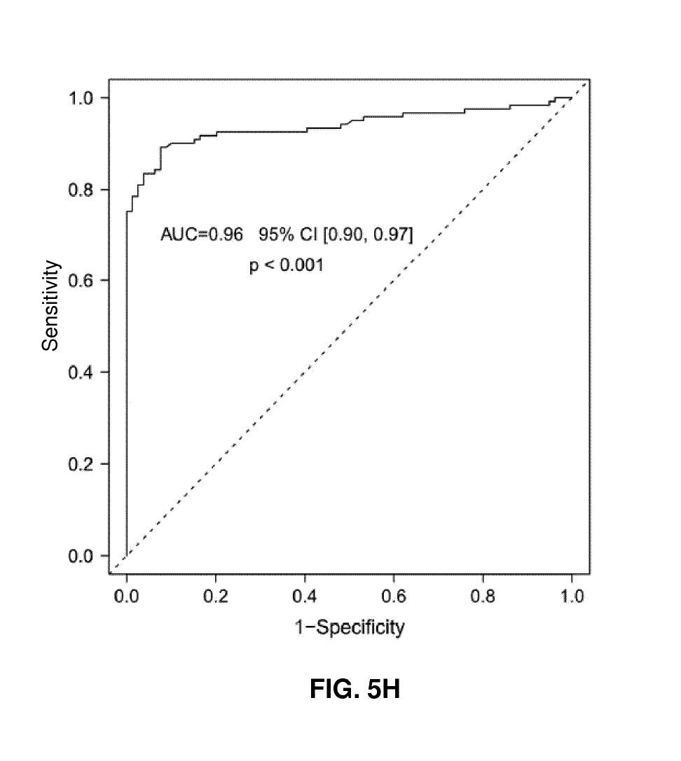

An independent cohort of FFPE primary tumors (122 stage I NSCLC and 79 non-lung cancer samples was recruited and DNA methylation levels for BCAT1, CDO1, TRIM55 and ZNF177 were determined by pyrosequencing. Clinical characteristics for this cohort are described in Table 8. The four biomarkers had significantly higher levels of DNA methylation in tumor samples as compared to non-tumoral controls (FIG. 5A-5D). Importantly, all the genes of the signature showed significant areas under the ROC curve (AUC) greater than 0.8 (AUC_{BCAT1}=0.94, AUC_{CDO1}=0.84, AUC_{TRIM58}=0.98 and AUC_{ZNF177}=0.96), suggesting a great diagnostic accuracy of these biomarkers for NSCLC detection (FIG. 5E-5H). Similarly, when samples were classified based on histological subtypes (ADC and SCC), the inventors observed for all the biomarkers significant differences in methylation level (data not shown) and AUCs close to 1.0 (AUC_{BCAT1}=0.94 (95% CI [0.91, 0.98] p<0.001); AUC_{CDO1}=0.87 (95% CI [0.79, 0.94] p<0.001); AUC_{TRIM55}=0.95 (95% CI [0.92, 0.99] p<0.001); AUC_{ZNF177}=0.95 (95% CI [0.87, 0.98] p<0.001) for ADC; AUC_{BCAT1}=0.94 (95% CI [0.91, 0.98] p<0.001); AUC_{CDO1}=0.81 (95% CI [0.72, 0.90] p<0.001); AUC_{TRIM55}=0.99 (95% CI [0.97, 1.0] p<0.001); AUC_{ZNF177}=0.99 (95% CI [0.91, 0.99] p<0.001) for SCC). Results from the four-gene epigenetic signature presented high diagnostic accuracy and were extremely similar to those obtained from public databases (data not shown) and FFPE samples, as shown in this Example. Importantly, the inventors analyzed a total of 79 non-tumoral control tissues, and DNA methylation was almost negligible in the vast amount of samples. These results confirmed the diagnostic value of evaluating DNA methylation levels by the method of the present invention, since even when minimally or non-invasive samples were used, in which the number of cancer cells is lower than in tissue samples, the results were the same as when using FFPE samples.

### Example 6.—Epigenetic Silencing of the Cancer-Specific Methylated Genes in Lung Cancer Primary Tumors

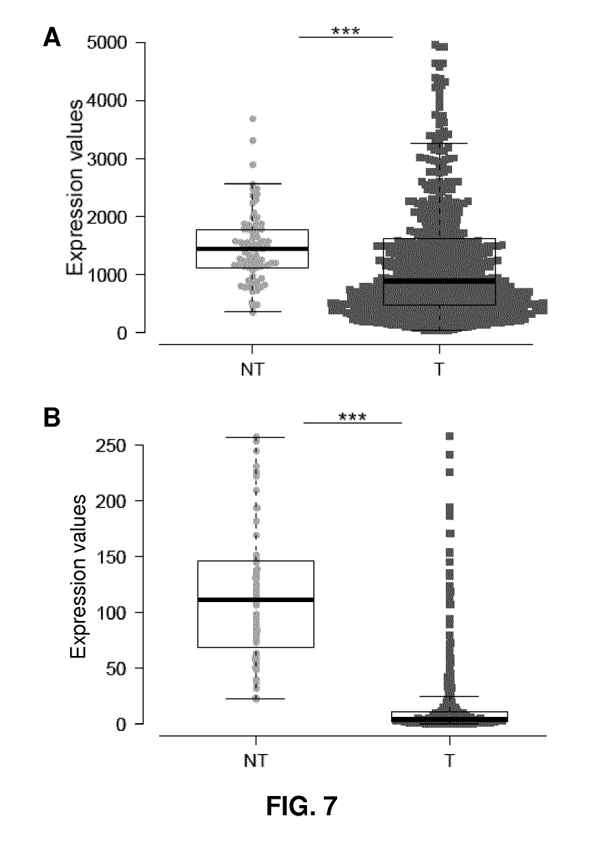

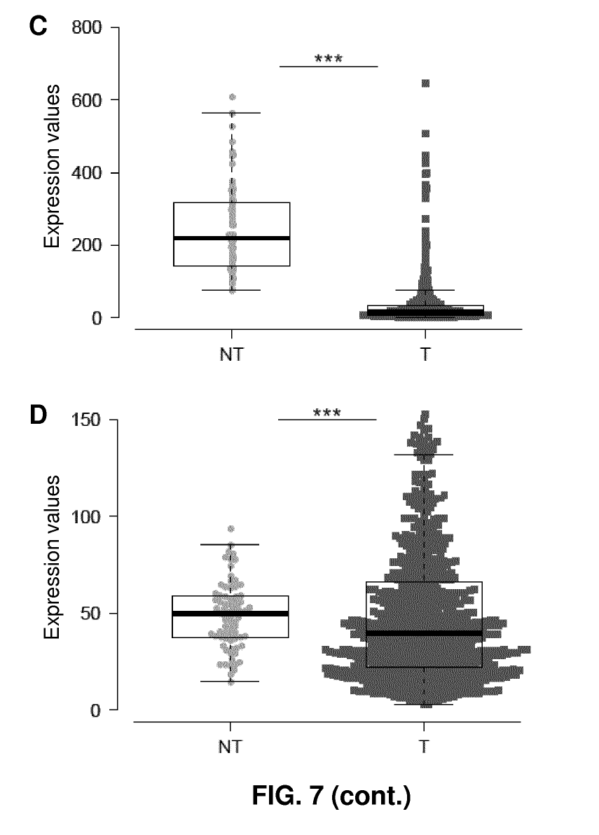

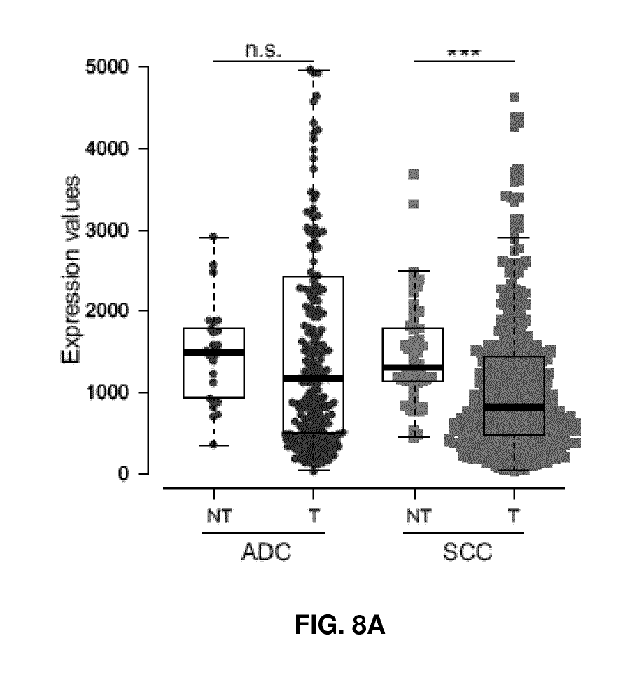

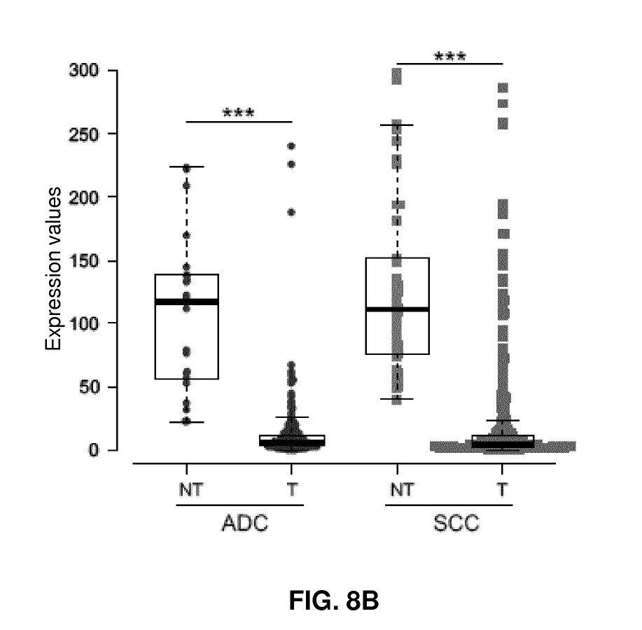

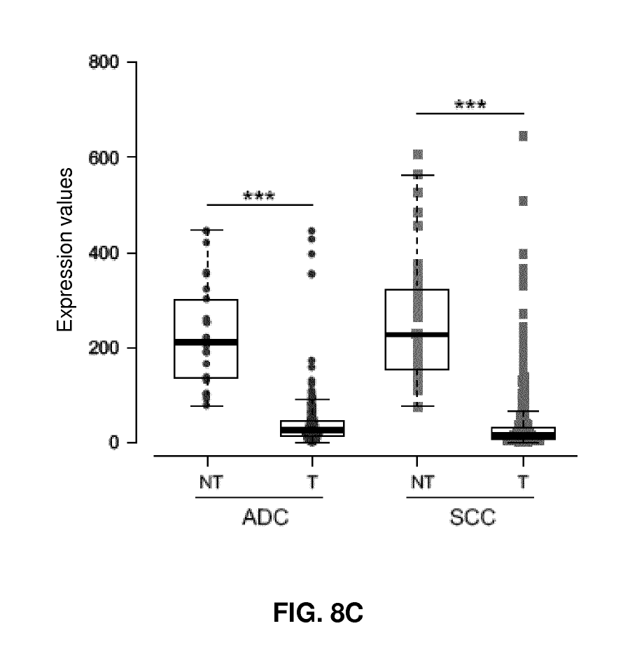

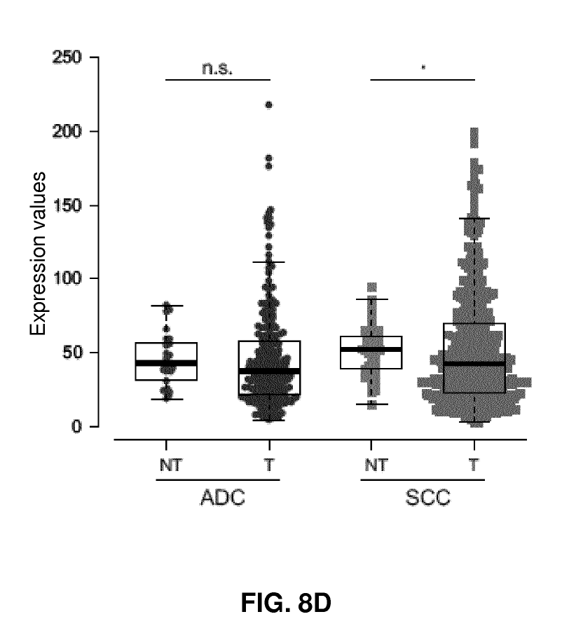

Promoter hypermethylation of multiple consecutive CpGs is recognized as an important mechanism by which genes may be silenced in both physiologically and pathological conditions. This mechanism for gene silencing has also been shown to play a relevant functional role in the development and progression of many common human tumors. In this regard, analyzing the CURELUNG FP7 publicly available dataset and TCGA (lung adenocarcinoma LUAD or Lung squamous cell carcinoma LUSC) datasets (Sandoval et al. J Clin Oncol 2013; 31:4140-7), the inventors observed a similar methylation pattern between the significant differentially DNA methylated CpGs (DMCpGs) of the selected biomarkers and their surrounding CpGs (FIG. 6). Importantly, gene expression analysis from the TCGA cohort samples showed a significantly decreased expression in BCAT1, CDO1, TRIM58 and ZNF177 (FIG. 7). Interestingly, expression results were also obtained for ADCs and SCCs separately (FIG. 8). These results reinforced the role of DNA methylation in the functional regulation of BCAT, CDO1, TRIM58 and ZNF177. Importantly, the data obtained suggest that the methylation values of these four genes represent an epigenetic signature that may be relevant in early steps of lung carcinogenesis.

1. An in vitro method for the diagnosis of lung cancer comprising the steps of:
a) determining the methylation level of a gene in a test sample, taken from a subject,
wherein the gene is one gene, a two-gene combination, a three-gene combination or a four-gene combination, and wherein the gene(s) is(are) selected from the group consisting of BCAT1, TRIM58, ZNF177 and CDO1, and at least one gene is BCAT1;
b) comparing the methylation level determined in step a) to a reference; and
c) identifying the subject as being likely to have lung cancer, if the methylation level of the test sample is higher than the methylation level of the reference, and identifying the subject as unlikely to have lung cancer if the methylation level of the test sample is below the methylation level of the reference.

2. The method according to claim 1, wherein the gene is a two-gene combination selected from BCAT1 and TRIM58; BCAT1 and CDO1; or BCAT1 and ZNF177.

3. The method according to claim 1, wherein the gene is a three-gene combination selected from BCAT1, TRIM58 and CDO1; BCAT1, TRIM58 and ZNF177;
or BCAT1, CDO1 and ZNF177.

4. The method according to claim 1, wherein the gene is a four-gene combination of BCAT1, TRIM58, CDO1 and ZNF177 genes.

5. The method according to claim 1, wherein BCAT1 gene comprises any one of SEQ ID 1-3, TRIM58 gene comprises any one of SEQ ID 4-8, CDO1 gene comprises any one of SEQ ID 9-11, and ZNF177 gene comprises any one of SEQ ID 12-15.

6. Method according to claim 1, wherein methylation is determined at one or more CpG site(s) and the position of the CpG site(s) is(are) selected from the group consisting of:
25054873, 25054905, 25055108, 25055214, 25055262, 25055304, 25055381, 25055421, 25055518, 25055676, 25055938, 25055948, 25055957, 25055959, 25055961, 25055967, 25055978, 25056083, 25056243, 25101448, 25102072, 25102274, 25102311, 25102431, 25102469, 25102521, 25103173 and 25103643 in BCAT1 gene,
248019234, 248019757, 248019816, 248020331, 248020350, 248020377, 248020436, 248020632, 248020641, 248020671, 248020680, 248020688, 248020692, 248020695, 248020697, 248020704, 248020707, 248020713, 248020812, 248021091 and 248021163 in TRIM58 gene,
115150172, 115151427, 115152019, 115152326, 115152386, 115152413, 115152420, 115152431, 115152485, 115152492, 115152466, 115152468, 115152475, 115152484, 115152494, 115152496, 115152503, 115152509, 115152522, 115152785, 115152835, 115152938 and 115153223 in CDO1 gene, and
9472210, 9473058, 9473240, 9473565, 9473598, 9473668, 9473674, 9473684, 9473688, 9473691, 9473696, 9473715, 9473715, 9473781, 9473880 and 9474128 in ZNF177 gene.

7. Method according to claim 1, wherein the methylation level is determined at one or more CpG site(s) selected from the group consisting of:
cg20399616 in BCAT1 gene,
cg23054189, cg07533148, cg20810478, cg16021909 in TRIM58 gene,
cg08065231 in ZNF177 gene, and
cg11036833 in CDO1 gene.

8. Method according to claim 1, wherein the methylation level is determined by bisulfite sequencing or by pyrosequencing, and preferably by pyrosequencing.

9. Method according to claim 1, wherein the test sample, taken from the subject, is selected from the group consisting of BAS, BAL, blood and sputum, and preferably is BAS.

10. A biomarker for in vitro lung cancer diagnosis, wherein the biomarker comprises a methylated gene, containing one or more methylated CpG site(s), wherein the gene is selected from one gene, a two-gene combination, a three-gene combination or a four-gene combination, and wherein the gene(s) is(are) selected from the group consisting of BCAT1, TRIM58, ZNF177 and CDO1, and at least one gene is BCAT1.

11. A kit to carry out the method according to claim 1, comprising:
primers for amplifying a CpG-containing nucleic acid of a gene, and/or
means for detecting the presence of methylated CpG site(s) in said amplified nucleic acid,
wherein the gene is selected from one gene, a two-gene combination, a three-gene combination or a four-gene combination, and
wherein the gene(s) is(are) selected from the group consisting of BCAT1, TRIM58, ZNF177 and CDO1, and at least one gene is BCAT1.

12. The biomarker according to claim 10, wherein BCAT1 gene comprises any one of SEQ ID 1-3, TRIM58 gene comprises any one of SEQ ID 4-8, CDO1 gene comprises any one of SEQ ID 9-11, and ZNF177 gene comprises any one of SEQ ID 12-15.

13. The biomarker according to claim 10, wherein the position of the CpG site(s) is(are) selected from the group consisting of:
25054873, 25054905, 25055108, 25055214, 25055262, 25055304, 25055381, 25055421, 25055518, 25055676, 25055938, 25055948, 25055957, 25055959, 25055961, 25055967, 25055978, 25056083, 25056243, 25101448, 25102072, 25102274, 25102311, 25102431, 25102469, 25102521, 25103173 and 25103643 in BCAT1,
248019234, 248019757, 248019816, 248020331, 248020350, 248020377, 248020436, 248020632, 248020641, 248020671, 248020680, 248020688, 248020692, 248020695, 248020697, 248020704, 248020707, 248020713, 248020812, 248021091 and 248021163 in TRIM58,
115150172, 115151427, 115152019, 115152326, 115152386, 115152413, 115152420, 115152431, 115152485, 115152492, 115152466, 115152468, 115152475, 115152484, 115152494, 115152496, 115152503, 115152509, 115152522, 115152785, 115152835, 115152938 and 115153223 in CDO1, and
9472210, 9473058, 9473240, 9473565, 9473598, 9473668, 9473674, 9473684, 9473688, 9473691, 9473696, 9473715, 9473715, 9473781, 9473880 and 9474128 in ZNF177.

14. The biomarker according to claim 10, wherein the one or more CpG site(s) is(are) selected from the group consisting of:
cg20399616 in BCAT1,
cg23054189, cg07533148, cg20810478, cg16021909 in TRIM58,
cg08065231 in ZNF177, and
cg11036833 in CDO1.

15. The method according to claim 1, configured for in vitro lung cancer diagnosis.
